(19) United States
(12) Patent Application Publication
(10) Pub. No.: US 2009/0258877 A1
(43) Pub. Date: Oct. 15, 2009

(54) TRIAZOLOTRIAZINES AND TRIAZOLOPYRAZINES AND THEIR USE

(75) Inventors: Stephan Siegel, Wuppertal (DE); Andreas Wilmen, Koln (DE); Niels Svenstrup, Velbert (DE); Mark Jean Gnoth, Mettmann (DE); Stefan Heitmeier, Wulfrath (DE); Ulrich Rester, Wupppertal (DE); Adrian Tersteegen, Wuppertal (DE); Michael Gerisch, Wuppertal (DE)

Correspondence Address: NIXON PEABODY LLP, 300 S. Riverside Plaza, 16th Floor, CHICAGO, IL, 60606-6613, US

(73) Assignee: BAYER HEALTHCARE AG, Leverkusen (DE)

(21) Appl. No.: 12/334,131

(22) Filed: Dec. 12, 2008

(30) Foreign Application Priority Data
Dec. 13, 2007 (DE) 102007060172.9
Jul. 29, 2008 (DE) 102008035209.8

(51) Int. Cl. A61K 31/53; C07D 471/04; C07D 487/04; A61K 31/498; A61P 7/00

(52) U.S. Cl. 514/243; 544/350; 544/184; 514/249

(57) ABSTRACT

The invention relates to substituted triazolotriazines and triazolopyrazines and to processes for their preparation, and also to their use for preparing medicaments for the treatment and/or prophylaxis of diseases, in particular haematologic disorders, preferably of leukopenias and neutropenias.

The invention relates to substituted triazolotriazines and triazolopyrazines and processes for their preparation, and their use for the manufacture of medicaments for the treatment and/or prophylaxis of diseases, in particular of haematological disorders, preferably of leukopenias and neutropenias.

Glycogen synthase kinase 3 (GSK3) belongs to the families of serine/threonin kinases. Specific substrates are inter alia cytoskeletal proteins and transcription factors. Two isoforms, GSK3α and GSK3β, have been identified to date (Woodgett JR., Trends Biochem. Sci. (1991), 16(5), 177-81). Both isoforms are constitutively active in chiefly resting, non-proliferating cells.

GSK3β is of central importance within the Wnt/Wingless signal transduction pathway. The latter is one of the most important, evolutionarily conserved signalling systems. Wnt signals control very early patterning processes during embryogenesis, they induce mesoderm formation and many organs, and they control the proliferation and differentiation of stem cells (Wodarz A., Nusse R., Annu. Rev. Cell Dev. Biol. (1998), 14, 59-88; Kirstetter et al., Nat. Immunol. (2006), 7(10), 1048-56). There is intracellular compartmentalization of the Wnt signalling pathway, thus making it possible to control a wide variety of processes. Within the Wnt cascade, glycogen synthase kinase 3 forms part of a multiprotein complex to which belong inter alia the structural molecules axin, the tumour suppressor protein APC and the transcription cofactor β-catenin. In this connection, β-catenin is the principal substrate of GSK3β. The consequence of this GSK3β-mediated phosphorylation is the proteasomal degradation of β-catenin. Inhibition of GSK3 activity leads to an accumulation of β-catenin in the cell with subsequent translocation into the cell nucleus. There, β-catenin acts as a cofactor in transcription complexes and thus is partly responsible for the expression of defined target genes.

Radiotherapies or chemotherapies are among the standard approaches to controlling cancer. Both types of therapy are nonspecific in relation to their target cells, i.e. not only tumour cells but also untransformed, proliferating cells are affected. These untransformed, proliferating cells also include haematopoietic progenitor cells which develop inter alia into neutrophilic granulocytes. A significant reduction in the number of neutrophils is referred to as neutropenia. A neutropenia induced by chemotherapy or radiotherapy results clinically in an increased susceptibility to infection. If the neutropenia is substantial there is an increase in the morbidity and, in some circumstances, also the mortality of a therapy (O'Brien et al., British Journal of Cancer (2006), 95, 1632-1636).

Inhibition of GSK3 activity leads to an increased rate of proliferation and differentiation of haematopoietic stem cells and can accordingly be utilized for therapeutic intervention in relation to a therapy-induced neutropenia.

WO2006/044687 describes inter alia triazolotriazines as kinase inhibitors for the treatment of cancer and disorders of the central nervous system. WO2007/138072 describes the use of 6-alkyl-substituted triazolopyrazines for the treatment of degenerative and inflammatory disorders.

One object of the present invention is therefore to provide novel compounds as GSK3β inhibitors for the treatment of haematological disorders, preferably of neutropenia in humans and animals.

The invention provides compounds of the formula

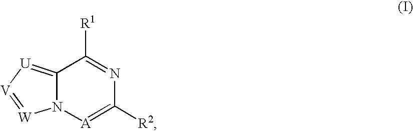

in which
either
U represents N,
V represents CR¹²,
W represents N,
A represents CR¹⁵,
or
U represents N,
V represents N,
W represents CR¹⁶,
A represents N.
where
R¹² represents hydrogen, hydroxyl, amino, hydroxycarbonyl, aminocarbonyl, trifluoromethyl, trifluoromethoxy, cyano, C₁-C₄-alkyl, C₁-C₄-alkoxy, C₁-C₄-alkylamino, C₁-C₄-alkylcarbonyl, C₁-C₄-alkoxycarbonyl, C₁-C₄-alkylaminocarbonyl, C₁-C₄-alkylcarbonylamino, C₁-C₄-alkylsulphonylamino, 5- or 6-membered heterocyclylcarbonyl, —CH₂R¹³ or —CH₂CH₂R¹⁴,
   where heterocyclylcarbonyl is substituted by 1 to 3 substituents, where the substituents independently of one another are selected from the group consisting of halogen, oxo, C₁-C₄-alkyl, C₁-C₄-alkoxy, C₁-C₄-alkylamino, C₁-C₄-alkylcarbonyl, C₁-C₄-alkoxycarbonyl and C₁-C₄-alkylaminocarbonyl,
   and
   where alkoxy, alkylamino, alkylcarbonyl, alkoxycarbonyl, alkylaminocarbonyl, alkylcarbonylamino and alkylsulphonylamino may be substituted by a substituent, where the substituent is selected from the group consisting of hydroxyl, amino, hydroxycarbonyl, aminocarbonyl, C₁-C₄-alkoxy, C₁-C₄-alkylamino, C₁-C₄-alkoxycarbonyl, C₁-C₄-alkylaminocarbonyl, C₁-C₄-alkylcarbonylamino, 5- or 6-membered heterocyclyl and phenyl,
      where phenyl may be substituted by 1 to 3 substituents, where the substituents independently of one another are selected from the group consisting of halogen, cyano, trifluoromethyl, trifluoromethoxy, aminocarbonyl, C₁-C₄-alkyl, C₁-C₄-alkoxy, C₁-C₄-alkylamino, C₁-C₄-alkylcarbonyl, C₁-C₄-alkoxycarbonyl, C₁-C₄-alkylaminocarbonyl and C₁-C₄-alkylcarbonylamino,
      and
      where heterocyclyl may be substituted by 1 to 3 substituents, where the substituents independently of one another are selected from the group consisting of halogen, oxo, C₁-C₄-alkyl, C₁-C₄-alkoxy, C₁-C₄-alkylamino, C₁-C₄-alkylcarbonyl, C₁-C₄-alkoxycarbonyl and C₁-C₄-alkylaminocarbonyl,
   and
   where
   R¹³ represents hydroxyl, amino, cyano, hydroxycarbonyl, aminocarbonyl, C₁-C₄-alkoxy, C₁-C₄-alkylamino, C₁-C₄-alkoxycarbonyl, C₁-C₄-alkylaminocarbonyl, C₁-C₄-alkylcarbonylamino or 5- or 6-membered heterocyclyl,
      where alkoxy, alkylamino, alkoxycarbonyl, alkylaminocarbonyl and alkylcarbonylamino may be substituted by a substituent, where the substituent is selected from the group consisting of hydroxyl, amino, hydroxycarbonyl, aminocarbonyl, C₁-C₄-alkoxy, C₁-C₄-alkylamino, C₁-C₄-alkoxycarbonyl, C₁-C₄-alkylaminocarbonyl and C₁-C₄-alkylcarbonylamino,
      and
      where heterocyclyl may be substituted by 1 to 3 substituents, where the substituents independently of one another are selected from the group consisting of halogen, oxo, C₁-C₄-alkyl, C₁-C₄-alkoxy, C₁-C₄-alkylamino, C₁-C₄-alkylcarbonyl, C₁-C₄-alkoxycarbonyl and C₁-C₄-alkylaminocarbonyl,
   and
   where
   R¹⁴ represents hydroxyl, amino, cyano, hydroxycarbonyl, aminocarbonyl, C₁-C₄-alkoxy, C₁-C₄-alkylamino, C₁-C₄-alkoxycarbonyl, C₁-C₄-alkylaminocarbonyl, C₁-C₄-alkylcarbonylamino or 5- or 6-membered heterocyclyl,
      where alkoxy, alkylamino, alkoxycarbonyl, alkylaminocarbonyl and alkylcarbonylamino may be substituted by a substituent, where the substituent is selected from the group consisting of hydroxyl, amino, hydroxycarbonyl, aminocarbonyl, C₁-C₄-alkoxy, C₁-C₄-alkylamino, C₁-C₄-alkoxycarbonyl, C₁-C₄-alkylaminocarbonyl and C₁-C₄-alkylcarbonylamino,
      and
      where heterocyclyl may be substituted by 1 to 3 substituents, where the substituents independently of one another are selected from the group consisting of halogen, oxo, C₁-C₄-alkyl, C₁-C₄-alkoxy, C₁-C₄-alkylamino, C₁-C₄-alkylcarbonyl, C₁-C₄-alkoxycarbonyl and C₁-C₄-alkylaminocarbonyl,
R¹⁵ represents hydrogen, halogen, cyano, trifluoromethyl, C₁-C₃-alkyl, methoxy, methylthio or cyclopropyl,
R¹⁶ represents hydrogen or methyl,
   R¹ represents a group of the formula

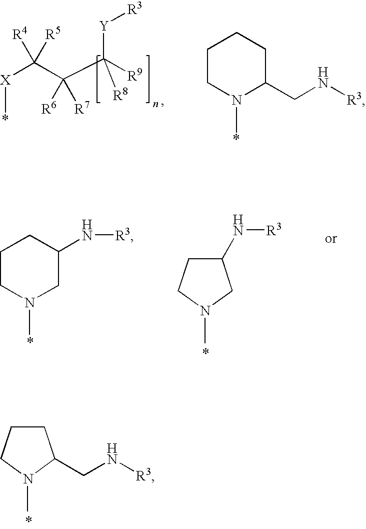

where
* is the point of attachment to the heterocycle,
n represents the number 0 or 1,
X represents NR¹⁰, S or O,
   where
   R¹⁰ represents hydrogen, C₁-C₃-alkyl or cyclopropyl,
Y represents NR¹¹ or S,
   where
   R¹¹ represents hydrogen, C₁-C₃-alkyl or cyclopropyl,
R³ represents pyrid-2-yl, pyrimid-2-yl, 2-aminopyrimid-4-yl, 2-(mono-C₁-C₄-alklamino)pyrimid-4-yl, 2-(mono-C₃-C₄-cycloalkylamino)pyrimid-4-yl, pyridazin-3(2H)-on-6-yl, 1,3-oxazol-2-yl, 1,3-oxazol-4-yl, 1,2,4-oxadiazol-3-yl, 1,2,3-oxadiazol-4-yl, 1,3-thiazol-2-yl, 1,3-thiazol-4-yl, 1H-1,2,4-triazol-5-yl, 2,4-dihydro-3H-1,2,4-triazol-3-on-5-yl or 1,2-pyrazol-5-yl,
   where pyrid-2-yl, pyrimid-2-yl, 1,3-oxazol-2-yl, 1,3-oxazol-4-yl, 1,3-thiazol-2-yl and 1,3-thiazol-4-yl are substituted by 1 or 2 substituents, where the substituents independently of one another are selected from the group consisting of halogen, cyano, nitro, amino, trifluoromethyl, trifluoromethoxy, aminocarbonyl, trifluoromethylcarbonyl, C₁-C₄-alkyl, C₁-C₄-alkoxy, C₁-C₄-alkylamino, C₃-C₄-cycloalkylamino, C₁-C₄-alkylcarbonyl, C₁-C₄-alkoxycarbonyl, C₁-C₄-alkylaminocarbonyl and C₃-C₆-cycloalkylcarbonyl,
      where alkyl, alkoxy, alkylamino, alkylcarbonyl, alkoxycarbonyl, alkylaminocarbonyl and cycloalkylcarbonyl may be substituted by a substituent, where the substituent is selected from the group consisting of halogen, cyano, hydroxyl, amino, trifluoromethyl and C₃-C₆-cycloalkyl,
   and
   where 2-aminopyrimid-4-yl, 2-(mono-C₁-C₄-alkylamino)pyrimid-4-yl, 2-(mono-C₃-C₄-cycloalkylamino)pyrimid-4-yl, pyridazin-3(2H)-on-6-yl, 1,2,4-oxadiazol-3-yl, 1,2,3-oxadiazol-4-yl, 1H-1,2,4-triazol-5-yl, 2,4-dihydro-3H-1,2,4-triazol-3-on-5-yl and 1,2-pyrazol-5-yl may be substituted by a substituent, where the substituent is selected from the group consisting of halogen, cyano, nitro, amino, trifluoromethyl, trifluoromethoxy, aminocarbonyl, trifluoromethylcarbonyl, C₁-C₄-alkyl, C₁-C₄-alkoxy, C₁-C₄-alkylamino, C₃-C₄-cycloalkylamino, C₁-C₄-alkoxycarbonyl, C₁-C₄-alkylaminocarbonyl and C₃-C₆-cycloalkylcarbonyl,
R⁴ represents hydrogen, C₁-C₃-alkyl or cyclopropyl,
R⁵ represents hydrogen or C₁-C₃-alkyl,
R⁶ represents hydrogen, C₁-C₃-alkyl or cyclopropyl,
R⁷ represents hydrogen or C₁-C₃-alkyl,
R⁸ represents hydrogen, C₁-C₃-alkyl or cyclopropyl,
R⁹ represents hydrogen or C₁-C₃-alkyl,
   R² represents C₆-C₁₀-aryl or 5- to 10-membered heteroaryl,
where aryl and heteroaryl may be substituted by 1 to 3 substituents, where the substituents independently of one another are selected from the group consisting of hydroxyl, hydroxymethyl, amino, halogen, cyano, trifluoromethyl, trifluoromethoxy, aminocarbonyl, C₁-C₄-alkyl, C₁-C₄-alkoxy, C₁-C₄-alkoxymethyl, C₁-C₄-alkylamino, C₁-C₄-alkylaminomethyl, C₁-C₄-alkylcarbonyl, C₁-C₄-alkoxycarbonyl, C₁-C₄-alkylaminocarbonyl, C₁-C₄-alkylcarbonylamino, C₁-C₄-alkylsulphonyl, C₁-C₄-alkylsulphonylamino, C₁-C₄-alkylaminosulphonyl, phenyl, benzyloxy, 5- or 6-membered heterocyclyl, 5- or 6-membered heterocyclylcarbonyl, 5- or 6-membered heterocyclylmethyl and 5- or 6-membered heteroaryl,
   where phenyl, benzyloxy, heterocyclyl, heterocyclylcarbonyl, heterocyclylmethyl and heteroaryl may be substituted by 1 to 3 substituents, where the substituents independently of one another are selected from the group consisting of halogen, cyano, trifluoromethyl, trifluoromethoxy, aminocarbonyl, C₁-C₄-alkyl, C₁-C₄-alkoxy, C₁-C₄-alkylamino, C₁-C₄-alkylcarbonyl, C₁-C₄-alkoxycarbonyl, C₁-C₄-alkylaminocarbonyl and C₁-C₄-alkylcarbonylamino,
or
two of the substituents on aryl together with the carbon atoms to which they are attached form a 1,3-dioxolane or 1,4-dioxane,
   and their salts, their solvates and the solvates of their salts.

Compounds according to the invention are the compounds of the formula (I) and the salts, solvates and solvates of the salts thereof, as well as the compounds encompassed by the formula (I) and mentioned below as exemplary embodiment(s), and the salts, solvates and solvates of the salts thereof, insofar as the compounds encompassed by formula (I) and mentioned below are not already salts, solvates and solvates of the salts.

The compounds of the invention may, depending on their structure, exist in stereoisomeric forms (enantiomers, diastereomers). The invention therefore encompasses the enantiomers or diastereomers and respective mixtures thereof. The stereoisomerically pure constituents can be isolated in a known manner from such mixtures of enantiomers and/or diastereomers.

Where the compounds of the invention can occur in tautomeric forms, the present invention encompasses all tautomeric forms.

Salts preferred for the purposes of the present invention are physiologically acceptable salts of the compounds of the invention. However, salts which are themselves unsuitable for pharmaceutical applications but can be used for example for isolating or purifying the compounds of the invention are also encompassed.

Physiologically acceptable salts of the compounds of the invention include acid addition salts of mineral acids, carboxylic acids and sulphonic acids, e.g. salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, trifluoroacetic acid, propionic acid, lactic acid, tartaric acid, malic acid, citric acid, fumaric acid, maleic acid and benzoic acid.

Physiologically acceptable salts of the compounds of the invention also include salts of conventional bases such as, for example and preferably, alkali metal salts (e.g. sodium and potassium salts), alkaline earth metal salts (e.g. calcium and magnesium salts) and ammonium salts derived from ammonia or organic amines having 1 to 16 C atoms, such as, for example and preferably, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine, N-methylpiperidine and choline.

Solvates refer for the purposes of the invention to those forms of the compounds of the invention which form a complex in the solid or liquid state through coordination with solvent molecules. Hydrates are a specific form of solvates in which the coordination takes place with water.

The present invention also encompasses prodrugs of the compounds according to the invention. The term “prodrugs” encompasses compounds which themselves may be biologically active or inactive but are converted during their residence time in the body into compounds according to the invention (for example by metabolism or hydrolysis).

For the purposes of the present invention, the substituents have, unless specified otherwise, the following meaning:

Alkyl per se and “alk” and “alkyl” in alkoxy, alkylamino, alkylcarbonyl alkoxycarbonyl alkylaminocarbonyl alkylcarbonylamino, alkylsulphonyl alkylsulphonylamino and alkylaminosulphonyl stand for a linear or branched alkyl radical having 1 to 4 carbon atoms, by way of example, and preferably for methyl, ethyl, n-propyl, isopropyl, n-butyl and tert-butyl.

Alkoxy stands by way of example and preferably for methoxy, ethoxy, n-propoxy, isopropoxy, n-butoxy and tert-butoxy.

Alkylamino stands for an alkylamino radical having one or two alkyl substituents (chosen independently of one another), by way of example and preferably for methylamino, ethylamino, n-propylamino, isopropylamino, tert-butylamino, N,N-dimethylamino, N,N-diethylamino, N-ethyl-N-methylamino, N-methyl-N-n-propylamino, N-isopropyl-N-n-propylamino and N-tert-butyl-N-methylamino. C₁-C₄-alkylamino stands for example for a monoalkylamino radical having 1 to 4 carbon atoms or for a dialkylamino radical having 1 to 4 carbon atoms in each alkyl substituent in each case.

Monoalkylamino stands for an alkylamino radical having a linear or branched alkyl substituent, by way of example and preferably for methylamino, ethylamino, n-propylamino, isopropylamino and tert-butylamino.

Monocycloalkylamino stands for a cycloalkylamino radical having a cycloalkyl substituent, where the other substituent at the amino radical is hydrogen, by way of example and preferably for cyclopropylamino and cyclobutylamino.

Alkylcarbonyl stands by way of example and preferably for methylcarbonyl, ethylcarbonyl, n-propyl-carbonyl, isopropylcarbonyl, n-butylcarbonyl and tert-butylcarbonyl.

Alkoxycarbonyl stands by way of example and preferably for methoxycarbonyl, ethoxycarbonyl, n-propoxycarbonyl, isopropoxycarbonyl, n-butoxycarbonyl and tert-butoxycarbonyl.

Alkylaminocarbonyl stands for an alkylaminocarbonyl radical having one or two alkyl substituents (chosen independently of one another), by way of example and preferably for methylaminocarbonyl, ethylaminocarbonyl, n-propylaminocarbonyl, isopropylaminocarbonyl, tert-butylaminocarbonyl, N,N-dimethylaminocarbonyl, N,N-diethylaminocarbonyl, N-ethyl-N-methylaminocarbonyl, N-methyl-N-n-propylaminocarbonyl, N-isopropyl-N-n-propylaminocarbonyl and N-tert-butyl-N-methylaminocarbonyl. C₁-C₄-Alkylaminocarbonyl stands for example for a monoalkylaminocarbonyl radical having 1 to 4 carbon atoms or for a dialkylaminocarbonyl radical having 1 to 4 carbon atoms in each alkyl substituent in each case.

Alkylcarbonylamino stands by way of example and preferably for methylcarbonylamino, ethyl-carbonylamino, n-propylcarbonylamino, isopropylcarbonylamino, n-butylcarbonylamino and tert-butylcarbonylamino.

Alkylsulphonyl stands by way of example and preferably for methylsulphonyl, ethylsulphonyl, n-propylsulphonyl, isopropylsulphonyl, n-butylsulphonyl and tert-butylsulphonyl.

Alkylaminosulphonyl stands for an alkylaminosulphonyl radical having one or two alkyl substituents (chosen independently of one another), by way of example and preferably for methylaminosulphonyl, ethylaminosulphonyl, n-propylaminosulphonyl, isopropylaminosulphonyl, tert-butylaminosulphonyl, N,N-dimethylaminosulphonyl, N,N-diethylaminosulphonyl, N-ethyl-N-methylaminosulphonyl, N-methyl-N-n-propylaminosulphonyl, N-isopropyl-N-n-propylaminosulphonyl and N-tert-butyl-N-methylaminosulphonyl. C₁-C₄-Alkylaminosulphonyl stands for example for a monoalkylaminosulphonyl radical having 1 to 4 carbon atoms or for a dialkylaminosulphonyl radical having 1 to 4 carbon atoms in each alkyl substituent in each case.

Alkylsulphonylamino stands by way of example and preferably for methylsulphonylamino, ethyl-sulphonylamino, n-propylsulphonylamino, isopropylsulphonylamino, n-butylsulphonylamino and tert-butylsulphonylamino.

Cycloalkyl stands for a monocyclic cycloalkyl group usually having 3 to 6 carbon atoms, and mention may be made by way of example and preferably of cyclopropyl, cyclobutyl, cyclopentyl and cyclohexyl for cycloalkyl.

Heterocyclyl stands for a monocyclic, heterocyclic radical having 5 or 6 ring atoms and up to 3, preferably up to 2 heteroatoms and/or heterogroups from the series N, O, S, SO, SO₂, where a nitrogen atom may also form an N-oxide. The heterocyclyl radicals may be saturated or partly unsaturated. 5- or 6-membered, monocyclic saturated heterocyclyl radicals having up to 2 heteroatoms from the series O, N and S are preferred, by way of example and preferably for pyrrolidin-2-yl, pyrrolidin-3-yl, pyrrolinyl, tetrahydrofuranyl, tetrahydrothienyl, pyranyl, piperidin-1-yl, piperidin-2-yl, piperidin-3-yl, piperidin-4-yl, thiopyranyl, morpholin-1-yl, morpholin-2-yl, morpholin-3-yl, piperazin-1-yl, piperazin-2-yl.

Heteroaryl stands for an aromatic, mono- or bicyclic radical usually having 5 to 10, preferably 5 or 6 ring atoms and up to 5, preferably up to 4 heteroatoms from the series S, O and N, where a nitrogen atom may also form an N-oxide, by way of example and preferably for thienyl, furyl, pyrrolyl, thiazolyl, oxazolyl, oxadiazolyl, pyrazolyl, imidazolyl, triazolyl, pyridyl, pyrimidyl, pyridazinyl, pyrazinyl, indolyl, indazolyl, benzofuranyl, benzothiophenyl, quinolinyl, isoquinolinyl, benzoxazolyl, benzimidazolyl.

Halogen stands for fluorine, chlorine, bromine and iodine, preferably for fluorine and chlorine.

In the formulae of the group which can stand for R¹, the end point of the line, besides which a # stands, in each case does not stand for a carbon atom or a CH₂ group but forms part of the bond to the atom to which R¹ is bonded.

In the formulae of the group which can stand for R³, the end point of the line, besides which a * stands, in each case does not stand for a carbon atom or a CH₂ group but forms part of the bond to the atom to which R³ is bonded.

Preference is given to compounds of the formula (I) in which

either
U represents N,
V represents CR¹²,
W represents N,
A represents CR¹⁵,
or
U represents N,
V represents N,
W represents CR¹⁶,
A represents N,
where
R¹² represents hydrogen, hydroxycarbonyl, aminocarbonyl, C₁-C₄-alkyl, C₁-C₄-alkoxy, C₁-C₄-alkylamino, C₁-C₄-alkylcarbonyl, C₁-C₄-alkoxycarbonyl, C₁-C₄-alkylaminocarbonyl, C₁-C₄-alkylcarbonylamino, 5- or 6-membered heterocyclylcarbonyl, —CH₂R¹³ or —CH₂ CH₂R¹⁴,
   where heterocyclylcarbonyl may be substituted by 1 to 3 substituents, where the substituents independently of one another are selected from the group consisting of oxo, C₁-C₄-alkyl, C₁-C₄-alkoxy, C₁-C₄-alkylamino, C₁-C₄-alkylcarbonyl, C₁-C₄-alkoxycarbonyl and C₁-C₄-alkylaminocarbonyl,
   and
   where alkoxy, alkylamino, alkylcarbonyl, alkoxycarbonyl, alkylaminocarbonyl and alkylcarbonylamino may be substituted by a substituent, where the substituent is selected from the group consisting of hydroxyl, amino, hydroxycarbonyl, aminocarbonyl, C₁-C₄-alkoxy, C₁-C₄-alkylamino and 5- or 6-membered heterocyclyl,
      where heterocyclyl may be substituted by 1 or 2 substituents, where the substituents independently of one another are selected from the group consisting of oxo, C₁-C₄-alkyl, C₁-C₄-alkoxy, C₁-C₄-alkylamino, C₁-C₄-alkylcarbonyl, C₁-C₄-alkoxycarbonyl and C₁-C₄-alkylaminocarbonyl,
   and
   where
   R¹³ represents hydroxyl, amino, hydroxycarbonyl, aminocarbonyl, C₁-C₄-alkoxy, C₁-C₄-alkylamino, C₁-C₄-alkoxycarbonyl, C₁-C₄-alkylaminocarbonyl, C₁-C₄-alkylcarbonylamino or 5- or 6-membered heterocyclyl,
      where alkoxy, alkylamino, alkoxycarbonyl, alkylaminocarbonyl and alkylcarbonylamino may be substituted by a substituent, where the substituent is selected from the group consisting of hydroxyl, amino, hydroxycarbonyl, aminocarbonyl, C₁-C₄-alkoxy, C₁-C₄-alkylamino, C₁-C₄-alkoxycarbonyl, C₁-C₄-alkylaminocarbonyl and C₁-C₄-alkylcarbonylamino,
      and
      where heterocyclyl may be substituted by 1 or 2 substituents, where the substituents independently of one another are selected from the group consisting of oxo, C₁-C₄-alkyl, C₁-C₄-alkoxy, C₁-C₄-alkylamino, C₁-C₄-alkylcarbonyl, C₁-C₄-alkoxycarbonyl and C₁-C₄-alkylaminocarbonyl,
   and
   where
   R¹⁴ represents hydroxyl, amino, hydroxycarbonyl, aminocarbonyl, C₁-C₄-alkoxy, C₁-C₄-alkylamino, C₁-C₄-alkoxycarbonyl, C₁-C₄-alkylaminocarbonyl, C₁-C₄-alkylcarbonylamino or 5- or 6-membered heterocyclyl,
      where alkoxy, alkylamino, alkoxycarbonyl, alkylaminocarbonyl and alkylcarbonylamino may be substituted by a substituent, where the substituent is selected from the group consisting of hydroxyl, amino, hydroxycarbonyl, aminocarbonyl, C₁-C₄-alkoxy, C₁-C₄-alkylamino, C₁-C₄-alkoxycarbonyl, C₁-C₄-alkylaminocarbonyl and C₁-C₄-alkylcarbonylamino,
      and
      where heterocyclyl may be substituted by 1 or 2 substituents, where the substituents independently of one another are selected from the group consisting of oxo, C₁-C₄-alkyl, C₁-C₄-alkoxy, C₁-C₄-alkylamino, C₁-C₄-alkylcarbonyl, C₁-C₄-alkoxycarbonyl and C₁-C₄-alkylaminocarbonyl,
R¹⁵ represents hydrogen, halogen, cyano or trifluoromethyl,
R¹⁶ represents hydrogen or methyl,
   R¹ represents a group of the formula

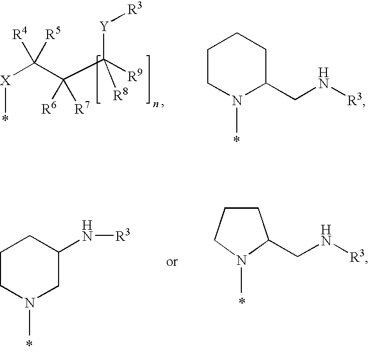

where
* is the point of attachment to the heterocycle,
n represents the number 0 or 1,
X represents NR¹⁰, S or O,
   where
   R¹⁰ represents hydrogen or methyl,
Y represents NR¹¹ or S,
   where
   R¹¹ represents hydrogen or methyl,
R³ represents pyrid-2-yl, pyrimid-2-yl, 2-aminopyrimid-4-yl, 1,3-oxazol-2-yl, 1,3-oxazol-4-yl, 1,2,4-oxadiazol-3-yl, 1,2,3-oxadiazol-4-yl, 1,3-thiazol-2-yl or 1,3-thiazol-4-yl,
   where pyrid-2-yl, pyrimid-2-yl, 1,3-oxazol-2-yl, 1,3-oxazol-4-yl, 1,3-thiazol-2-yl and 1,3-thiazol-4-yl are substituted by 1 or 2 substituents, where the substituents independently of one another are selected from the group consisting of halogen, cyano, nitro, amino, trifluoromethyl, trifluoromethoxy, aminocarbonyl, trifluoromethylcarbonyl, methyl, ethyl, methoxy, ethoxy, C₁-C₄-alkylamino, methylcarbonyl, ethylcarbonyl, cyclopropylcarbonyl, methoxycarbonyl and ethoxycarbonyl,
   and
   where 2-aminopyrimid-4-yl, 1,2,4-oxadiazol-3-yl and 1,2,3-oxadiazol-4-yl may be substituted by a substituent, where the substituent is selected from the group consisting of halogen, cyano, nitro, amino, trifluoromethyl, trifluoromethoxy, aminocarbonyl, trifluoromethylcarbonyl, methyl, ethyl, methoxy, ethoxy, C₁-C₄-alkylamino, methylcarbonyl, ethylcarbonyl, cyclopropylcarbonyl, methoxycarbonyl and ethoxycarbonyl,
R⁴ represents hydrogen or methyl,
R⁵ represents hydrogen or methyl,
R⁶ represents hydrogen or methyl,
R⁷ represents hydrogen or methyl,
R⁸ represents hydrogen or methyl,
R⁹ represents hydrogen or methyl,
R² represents C₆-C₁₀-aryl, thienyl, furyl, pyrrolyl, thiazolyl, oxazolyl, oxadiazolyl, pyrazolyl, imidazolyl, pyridyl, pyrimidyl, pyridazinyl, pyrazinyl, indolyl, indazolyl, quinolinyl, benzofuranyl or benzoxazolyl,
   where aryl, thienyl, furyl, pyrrolyl, thiazolyl, oxazolyl, oxadiazolyl, pyrazolyl, imidazolyl, pyridyl, pyrimidyl, pyridazinyl, pyrazinyl, indolyl, indazolyl, quinolinyl, benzofuranyl and benzoxazolyl may be substituted by 1 to 3 substituents, where the substituents independently of one another are selected from the group consisting of hydroxyl, hydroxymethyl, amino, halogen, cyano, trifluoromethyl, trifluoromethoxy, aminocarbonyl, C₁-C₄-alkyl, C₁-C₄-alkoxy, C₁-C₄-alkoxymethyl, C₁-C₄-alkylamino, C₁-C₄-alkylaminomethyl, C₁-C₄-alkyl-carbonyl, C₁-C₄-alkoxycarbonyl, C₁-C₄-alkylaminocarbonyl, C₁-C₄-alkylcarbonylamino, C₁-C₄-alkylsulphonyl, C₁-C₄-alkylsulphonylamino, C₁-C₄-alkylaminosulphonyl, phenyl, benzyloxy, 5- or 6-membered heterocyclyl, 5- or 6-membered heterocyclylcarbonyl, 5- or 6-membered heterocyclylmethyl and 5- or 6-membered heteroaryl,
      where phenyl, benzyloxy, heterocyclyl, heterocyclylcarbonyl, heterocyclylmethyl and heteroaryl may be substituted by 1 to 3 substituents, where the substituents independently of one another are selected from the group consisting of halogen, cyano, trifluoromethyl, trifluoromethoxy, aminocarbonyl, C₁-C₄-alkyl, C₁-C₄-alkoxy, C₁-C₄-alkylamino, C₁-C₄-alkylcarbonyl, C₁-C₄-alkoxycarbonyl, C₁-C₄-alkylaminocarbonyl and C₁-C₄-alkylcarbonylamino,
         and their salts, their solvates and the solvates of their salts.

Preference is also given to compounds of the formula (I) in which

either
U represents N,
V represents CR¹²,
W represents N,
A represents CR¹⁵,
or
U represents N,
V represents N,
W represents CR¹⁶,
A represents N,
where
R¹² represents hydrogen, hydroxycarbonyl, aminocarbonyl, methyl, ethyl, C₁-C₄-alkylcarbonyl, C₁-C₄-alkoxycarbonyl, C₁-C₄-alkylaminocarbonyl, C₁-C₄-alkylcarbonylamino, pyrrolidinylcarbonyl, piperidinylcarbonyl, piperazinylcarbonyl, morpholinylcarbonyl or —CH₂R¹³,
   where pyrrolidinylcarbonyl, piperidinylcarbonyl, piperazinylcarbonyl and morpholinylcarbonyl may be substituted by 1 or 2 substituents, where the substituents independently of one another are selected from the group consisting of oxo, methyl and ethyl,
   and
   where alkylcarbonyl, C₂-C₄-alkoxycarbonyl and C₂-C₄-alkylaminocarbonyl may be substituted by a substituent, where the substituent is selected from the group consisting of hydroxyl, amino, C₁-C₄-alkylamino, pyrrolidinyl, piperidinyl, piperazinyl and morpholinyl,
      where pyrrolidinyl, piperidinyl, piperazinyl and morpholinyl may be substituted by 1 or 2 substituents, where the substituents independently of one another are selected from the group consisting of oxo, methyl and ethyl,
   and
   where
   R¹³ represents hydroxyl, amino, hydroxycarbonyl, aminocarbonyl, C₁-C₄-alkoxy, C₁-C₄-alkylamino, pyrrolidinyl, piperidinyl, piperazinyl or morpholinyl,
      where pyrrolidinyl, piperidinyl, piperazinyl and morpholinyl may be substituted by 1 or 2 substituents, where the substituents independently of one another are selected from the group consisting of oxo, methyl and ethyl,
R¹⁵ represents hydrogen,
R¹⁶ represents hydrogen,
   R¹ represents a group of the formula

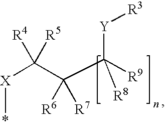

where
* is the point of attachment to the heterocycle,
n represents the number 0,
X represents NR¹⁰,
   where
   R¹⁰ represents hydrogen,
Y represents NR¹¹,
   where
   R¹¹ represents hydrogen,
R³ represents pyrid-2-yl, pyrimid-2-yl, 2-aminopyrimid-4-yl, 1,3-thiazol-2-yl or 1,3-thiazol-4-yl,
   where pyrid-2-yl, pyrimid-2-yl, 1,3-thiazol-2-yl and 1,3-thiazol-4-yl are substituted by 1 or 2 substituents, where the substituents independently of one another are selected from the group consisting of fluorine, chlorine, cyano, nitro, amino, trifluoromethyl and trifluoromethylcarbonyl,
   and
   where 2-aminopyrimid-4-yl may be substituted by a substituent, where the substituent is selected from the group consisting of fluorine, chlorine, cyano, nitro, amino, trifluoromethyl and trifluoromethylcarbonyl,
R⁴ represents hydrogen,
R⁵ represents hydrogen or methyl,
R⁶ represents hydrogen,
R⁷ represents hydrogen or methyl,
R⁸ represents hydrogen,
R⁹ represents hydrogen or methyl,
   R² represents phenyl, thienyl, pyrazolyl or pyridyl,
where phenyl, thienyl, pyrazolyl and pyridyl may be substituted by 1 or 2 substituents, where the substituents independently of one another are selected from the group consisting of halogen, trifluoromethyl, trifluoromethoxy, aminocarbonyl, C₁-C₄-alkyl, C₁-C₄-alkoxy, C₁-C₄-alkoxycarbonyl, C₁-C₄-alkylaminocarbonyl, pyrrolidinyl, piperidinyl, morpholinyl and morpholinylcarbonyl,
   and their salts, their solvates and the solvates of their salts.

Particular preference is given to compounds of the formula (I) in which

either
U represents N,
V represents CR¹²,
W represents N,
A represents CR¹⁵,
or
U represents N,
V represents N,
W represents CR¹⁶,
A represents N.
where
R¹² represents hydrogen, hydroxycarbonyl, methyl, ethyl, methoxycarbonyl, ethoxycarbonyl, C₁-C₄-alkylaminocarbonyl, piperidinylcarbonyl or morpholinylcarbonyl,
   where piperidinylcarbonyl and morpholinylcarbonyl may be substituted by a substituent, where the substituent is selected from the group consisting of methyl and ethyl,
   and
   where C₂-C₄-alkylaminocarbonyl may be substituted by a substituent, where the substituent is selected from the group consisting of C₁-C₄-alkylamino, piperazinyl and morpholinyl,
   where piperazinyl and morpholinyl may be substituted by a substituent, where the substituent is selected from the group consisting of methyl and ethyl,
R¹⁵ represents hydrogen,
R¹⁶ represents hydrogen,
   R¹ represents a group of the formula

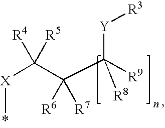

where
* is the point of attachment to the heterocycle,
n represents the number 0,
X represents NR¹⁰,
   where
   R¹⁰ represents hydrogen,
Y represents NR¹¹,
   where
   R¹¹ represents hydrogen,
R³ represents a group of the formula

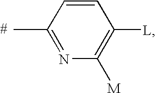

where
# is the point of attachment to Y,
L represents cyano, nitro, trifluoromethyl or trifluoromethylcarbonyl,
M represents hydrogen or amino,
R⁴ represents hydrogen,
R⁵ represents hydrogen or methyl,
R⁶ represents hydrogen,
R⁷ represents hydrogen or methyl,
R⁸ represents hydrogen,
R⁹ represents hydrogen,
   R² represents phenyl,
where phenyl may be substituted by 1 or 2 substituents, where the substituents independently of one another are selected from the group consisting of fluorine, chlorine, trifluoromethyl, trifluoromethoxy, C₁-C₃-alkyl, methoxy, methoxycarbonyl and ethoxycarbonyl,
   and their salts, their solvates and the solvates of their salts.

Particular preference is given to compounds of the formula (I) in which

either
U represents N,
V represents CR¹²,
W represents N,
A represents CR¹⁵,
or
U represents N,
V represents N,
W represents CR¹⁶,
A represents N,
where
R¹² represents hydrogen,
R¹⁵ represents hydrogen,
R¹⁶ represents hydrogen,
   R¹ represents a group of the formula

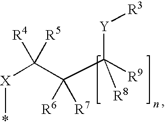

where
* is the point of attachment to the heterocycle,
n represents the number 0,
X represents NR¹⁰,
   where
   R¹⁰ represents hydrogen,
Y represents NR¹¹,
   where
   R¹¹ represents hydrogen,
R³ represents a group of the formula

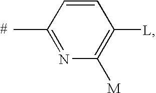

where
# is the point of attachment to Y,
either
L represents cyano,
and
M represents hydrogen,
or
L represents cyano, nitro or trifluoromethylcarbonyl,
and
M represents amino,
R⁴ represents hydrogen,
R⁵ represents hydrogen,
R⁶ represents hydrogen,
R⁷ represents hydrogen,
R⁸ represents hydrogen,
R⁹ represents hydrogen,
   R² represents phenyl,
where phenyl is substituted by 1 or 2 substituents, where the substituents independently of one another are selected from the group consisting of fluorine, chlorine and trifluoromethyl,
   and their salts, their solvates and the solvates of their salts.

Preference is also give to compounds of the formula (I) in which

either
U represents N,
V represents CR¹²,
W represents N,
A represents CR¹⁵,
or
U represents N,
V represents N,
W represents CR¹⁶,
A represents N,
where
R¹² represents hydrogen, hydroxycarbonyl, aminocarbonyl, methyl, ethyl, C₁-C₄-alkylcarbonyl, C₁-C₄-alkoxycarbonyl, C₁-C₄-alkylaminocarbonyl, C₁-C₄-alkyl-carbonylamino, pyrrolidinylcarbonyl, piperidinylcarbonyl, piperazinylcarbonyl, morpholinylcarbonyl or —CH₂R¹³,
   where pyrrolidinylcarbonyl, piperidinylcarbonyl, piperazinylcarbonyl and morpholinylcarbonyl may be substituted by 1 or 2 substituents, where the substituents independently of one another are selected from the group consisting of oxo, methyl and ethyl,
   and
   where alkylcarbonyl, C₂-C₄-alkoxycarbonyl and C₂-C₄-alkylaminocarbonyl may be substituted by a substituent, where the substituent is selected from the group consisting of hydroxyl, amino, C₁-C₄-alkylamino, pyrrolidinyl, piperidinyl, piperazinyl and morpholinyl,
      where pyrrolidinyl, piperidinyl, piperazinyl and morpholinyl may be substituted by 1 or 2 substituents, where the substituents independently of one another may be selected from the group consisting of oxo, methyl and ethyl,
   and
   where
   R¹³ represents hydroxyl, amino, hydroxycarbonyl, aminocarbonyl, C₁-C₄-alkoxy, C₁-C₄-alkylamino, pyrrolidinyl, piperidinyl, piperazinyl or morpholinyl,
      where pyrrolidinyl, piperidinyl, piperazinyl and morpholinyl may be substituted by 1 or 2 substituents, where the substituents independently of one another are selected from the group consisting of oxo, methyl and ethyl,
R¹⁵ represents hydrogen,
R¹⁶ represents hydrogen,
   R¹ represents a group of the formula

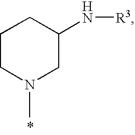

where
* is the point of attachment to the heterocycle,
R³ represents pyrid-2-yl, pyrimid-2-yl, 2-aminopyrimid-4-yl, 1,3-thiazol-2-yl or 1,3-thiazol-4-yl,
   where pyrid-2-yl, pyrimid-2-yl, 1,3-thiazol-2-yl and 1,3-thiazol-4-yl are substituted by 1 or 2 substituents, where the substituents independently of one another are selected from the group consisting of fluorine, chlorine, cyano, nitro, amino, trifluoromethyl and trifluoromethylcarbonyl,
   and
   where 2-aminopyrimid-4-yl may be substituted by a substituent, where the substituent is selected from the group consisting of fluorine, chlorine, cyano, nitro, amino, trifluoromethyl and trifluoromethylcarbonyl,
      R² represents phenyl, thienyl, pyrazolyl or pyridyl,
where phenyl, thienyl, pyrazolyl and pyridyl may be substituted by 1 or 2 substituents, where the substituents independently of one another are selected from the group consisting of halogen, trifluoromethyl, trifluoromethoxy, aminocarbonyl, C₁-C₄-alkyl, C₁-C₄-alkoxy, C₁-C₄-alkoxycarbonyl, C₁-C₄-alkylaminocarbonyl, pyrrolidinyl, piperidinyl, morpholinyl and morpholinylcarbonyl,
   and their salts, their solvates and the solvates of their salts.

Particular preference is given to compounds of the formula (I) in which

either
U represents N,
V represents CR¹²,
W represents N,
A represents CR¹⁵,
or
U represents N,
V represents N,
W represents CR¹⁶,
A represents N.
where
R¹² represents hydrogen, hydroxycarbonyl, methyl, ethyl, methoxycarbonyl, ethoxycarbonyl, C₁-C₄-alkylaminocarbonyl, piperidinylcarbonyl or morpholinylcarbonyl,
   where piperidinylcarbonyl and morpholinylcarbonyl may be substituted by a substituent, where the substituent is selected from the group consisting of methyl and ethyl,
   and
   where C₂-C₄-alkylaminocarbonyl may be substituted by a substituent, where the substituent is selected from the group consisting of C₁-C₄-alkylamino, piperazinyl and morpholinyl,
      where piperazinyl and morpholinyl may be substituted by a substituent, where the substituent is selected from the group consisting of methyl and ethyl,
R¹⁵ represents hydrogen,
R¹⁶ represents hydrogen,
   R¹ represents a group of the formula

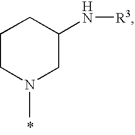

where
* is the point of attachment to the heterocycle,
R³ represents a group of the formula

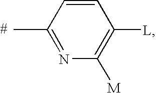

where
# is the point of attachment to NH,
L represents cyano, nitro, trifluoromethyl or trifluoromethylcarbonyl,
M represents hydrogen or amino,
   R² represents phenyl,
where phenyl may be substituted by 1 or 2 substituents, where the substituents independently of one another are selected from the group consisting of fluorine, chlorine, trifluoromethyl, trifluoromethoxy, C₁-C₃-alkyl, methoxy, methoxycarbonyl and ethoxycarbonyl,
   and their salts, their solvates and the solvates of their salts.

Particular preference is given to compounds of the formula (I) in which

either
U represents N,
V represents CR¹²,
W represents N,
A represents CR¹⁵,
or
U represents N,
V represents N,
W represents CR¹⁶,
A represents N,
where
R¹² represents hydrogen,
R¹⁵ represents hydrogen,
R¹⁶ represents hydrogen,
   R¹ represents a group of the formula

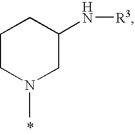

where
* is the point of attachment to the heterocycle,
R³ represents a group of the formula

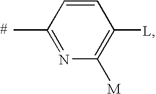

where
# is the point of attachment to NH,
either
L represents cyano,
and
M represents hydrogen,
or
L represents cyano, nitro or trifluoromethylcarbonyl,
and
M represents amino,
   R² represents phenyl,
where phenyl is substituted by 1 or 2 substituents, where the substituents independently of one another are selected from the group consisting of fluorine, chlorine and trifluoromethyl,
   and their salts, their solvates and the solvates of their salts.

Preference is also given to compounds of the formula (I) in which U represents N, V represents CR¹², W represents N and A represents CR¹⁵,

where
R¹² represents hydrogen, hydroxycarbonyl, methyl, ethyl, methoxycarbonyl, ethoxycarbonyl, C₁-C₄-alkylaminocarbonyl, piperidinylcarbonyl or morpholinylcarbonyl,
   where piperidinylcarbonyl and morpholinylcarbonyl may be substituted by a substituent, where the substituent is selected from the group consisting of methyl and ethyl,
   and
   where C₂-C₄-alkylaminocarbonyl may be substituted by a substituent, where the substituent is selected from the group consisting of C₁-C₄-alkylamino, piperazinyl and morpholinyl,
      where piperazinyl and morpholinyl may be substituted by a substituent, where the substituent is selected from the group consisting of methyl and ethyl,
         and
         R¹⁵ represents hydrogen.

Preference is also given to compounds of the formula (I) in which U represents N, V represents CR¹², W represents N and A represents CR¹⁵, where R¹² and R¹⁵ represent hydrogen.

Preference is also given to compounds of the formula (I) in which U represents N, V represents N, W represents CR¹⁶ and A represents N, where R¹⁶ represents hydrogen.

Preference is also given to compounds of the formula (I) in which R¹ represents —NHCH₂CH₂NH—R³.

Preference is also given to compounds of the formula (I) in which R¹ represents a group of the formula

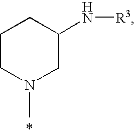

where * is the point of attachment to the heterocycle.

Preference is also given to compounds of the formula (I) in which R¹ represents —NHCH₂CH₂NH—R³, where R³ represents 5-cyanopyrid-2-yl.

Preference is also given to compounds of the formula (I) in which R¹ represents —NHCH₂CH₂NH—R³, where R³ represents 5-trifluoromethylcarbonyl-6-aminopyrid-2-yl.

Preference is also given to compounds of the formula (I) in which n represents the number 0.

Preference is also given to compounds of the formula (I) in which X represents NR¹⁰, where R¹⁰ represents hydrogen and Y represents NR¹¹, where R¹¹ represents hydrogen or methyl.

Preference is also given to compounds of the formula (I) in which X represents NR¹⁰, where ¹⁰ represents hydrogen.

Preference is also given to compounds of the formula (I) in which Y represents NR¹¹, where R¹¹ represents hydrogen.

Preference is also given to compounds of the formula (I) in which
R³ represents pyrid-2-yl, pyrimid-2-yl, 2-aminopyrimid-4-yl, 1,3-thiazol-2-yl or 1,3-thiazol-4-yl,
   where pyrid-2-yl, pyrimid-2-yl, 1,3-thiazol-2-yl and 1,3-thiazol-4-yl are substituted by 1 or 2 substituents, where the substituents independently of one another are selected from the group consisting of fluorine, chlorine, cyano, nitro, amino and trifluoromethyl,
   and
   where 2-aminopyrimid-4-yl may be substituted by a substituent, where the substituent is selected from the group consisting of fluorine, chlorine, cyano, nitro, amino and trifluoromethyl.

Preference is also given to compounds of the formula (I) in which

R³ represents a group of the formula

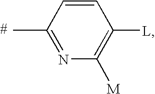

where
# is the point of attachment to Y or NH,
L represents cyano, nitro or trifluoromethyl,
M represents hydrogen or amino.

Preference is also given to compounds of the formula (I) in which R³ represents 5-cyanopyrid-2-yl.

Preference is also given to compounds of the formula (I) in which R³ represents 5-trifluoromethylcarbonyl-6-aminopyrid-2-yl.

Preference is also given to compounds of the formula (I) in which R⁴, R⁵, R⁶, R⁷, R⁸ and R⁹ represent hydrogen.

The invention furthermore provides a process for preparing compounds of the formula (I), or their salts, their solvates or the solvates of their salts, where

the compounds of the formula

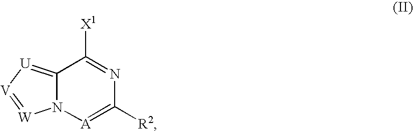

in which
A, U, V, W and R² have the meaning above,
and
X¹ represents halogen, preferably chlorine or fluorine,
are reacted with compounds of the formula

R¹—H (III)

in which
R¹ has the meaning given above.

The reaction is generally carried out in inert solvents, where appropriate in the presence of a base, where appropriate in a microwave, preferably in a temperature range from 50° C. to 200° C. under atmospheric pressure up to 5 bar.

Examples of bases are alkali metal carbonates, such as, for example, sodium carbonate, potassium carbonate or caesium carbonate, or organic bases, such as trialkylamines, for example triethylamine, N-methylmorpholine, N-methylpiperidine, 4-dimethylaminopyridine or diisopropylethylamine, or other bases, such as, for example, sodium hydride or potassium tert-butoxide; preference is given to diisopropylethylamine or sodium hydride.

Examples of inert solvents are halogenated hydrocarbons, such as methylene chloride or trichloromethane, alcohols, such as methanol, ethanol, n-propanol or isopropanol, or ethers, such as dioxane or tetrahydrofuran, or other solvents, such as, for example, dimethyl sulphoxide, dimethylformamide or N-methylpyrrolidone, or mixtures of these solvents; preference is given to N-methylpyrrolidone or dimethyl sulphoxide.

The compounds of the formula (II) are known, they can be synthesized by known processes from the appropriate starting materials or they can be prepared analogously to processes described in the example section (Examples 3A to 10A and Examples 18A to 20A) or analogously to J. Org. Chem. (2005), 70 (18), 7331-7337 and WO 03/000693.

The compounds of the formula (III) are known, they can be synthesized by known processes from the appropriate starting materials or they can be prepared analogously to the processes described in the example section (Examples 1A and 2A, Examples 11A to 17A and Examples 21A to 24A).

The preparation of the starting materials and the compounds of the formula (I) can be illustrated by the synthesis schemes below.

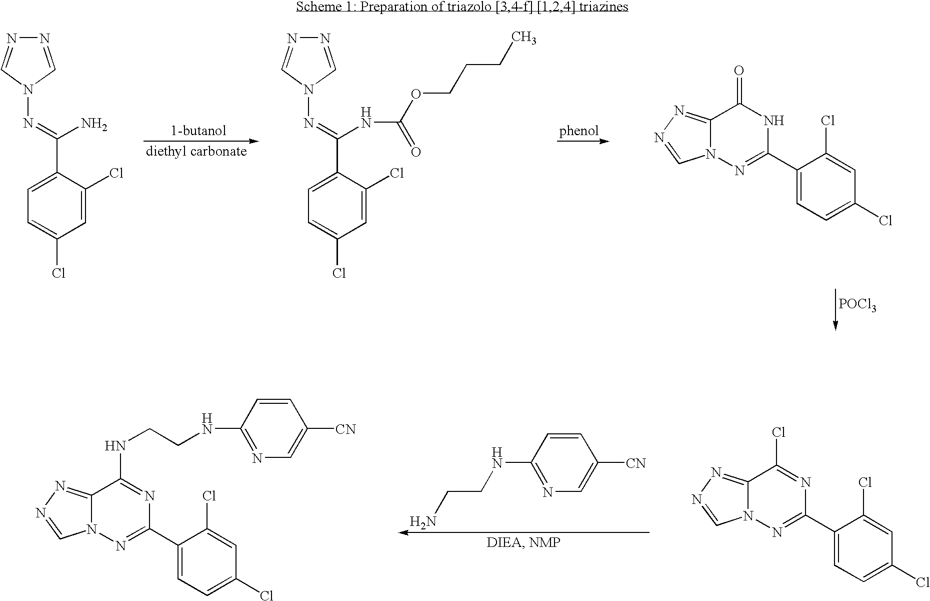

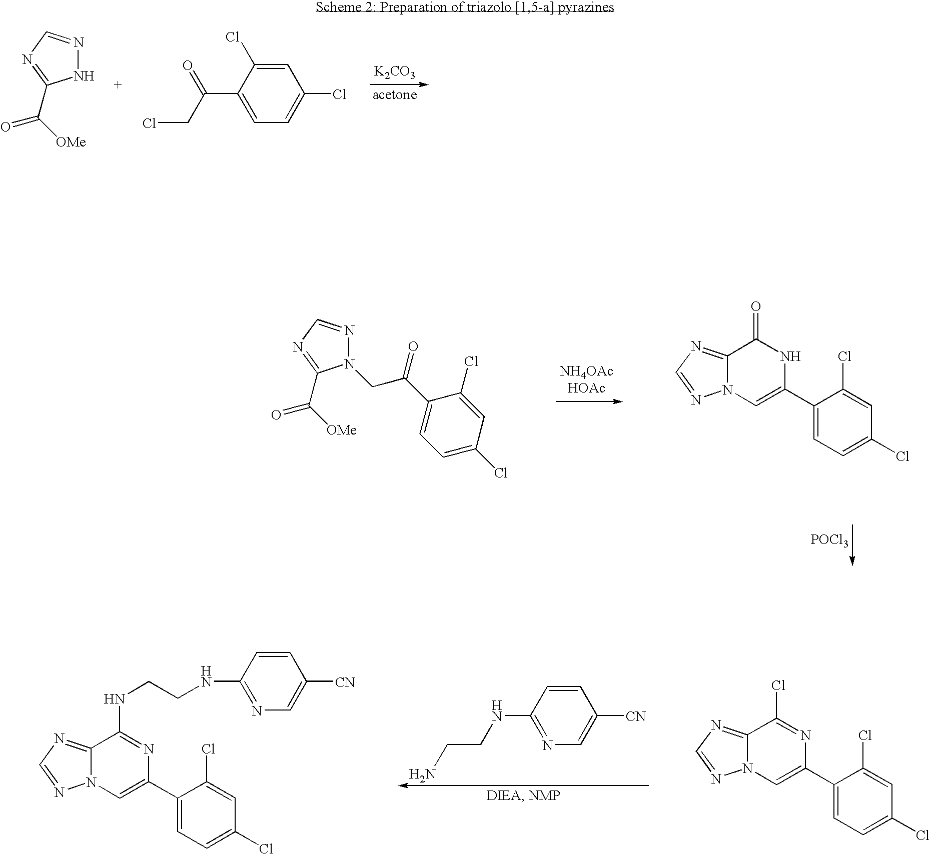

The compounds according to the invention show a valuable range of pharmacological and pharmacokinetic effects which could not have been predicted.

They are therefore suitable of use as medicaments for the treatment and/or prophylaxis of diseases in humans and animals.

The present invention further relates to the use of the compounds according to the invention for the treatment and/or prophylaxis of disorders, preferably haematological disorders, especially of leukopenias and neutropenia.

The compounds according to the invention are therefore suitable for the prophylaxis and/or treatment of neurodegenerative disorders such as, for example, Alzheimer's, Parkinson's, schizophrenia, degeneration, dementia, depression, aggression, cerebrovascular ischaemia, sleep disorders, Huntington's chorea, neurotraumatic disorders such as, for example, stroke; type 2 diabetes mellitus and associated disorders such as, for example, the metabolic syndrome or obesity, type 1 diabetes mellitus, diabetic nephropathy, diabetic neuropathy, diabetic retinopathy, glomerulonephritis, hypercalcaemia, hyperglycaemia, hyperlipidaemia, glucose-galactose malabsorption, general endocrine dysfunctions such as, for example, pancreatitis; haematological disorders such as, for example, acquired and congenital neutropenia, medicament-induced neutropenia, parasite-induced neutropenia, chemotherapy-induced neutropenia, granulocytopenia, acquired and congenital leucopenia, acquired and congenital anaemia, haemolytic anaemia, sickle cell anaemia, acquired and congenital thrombocytopenia, leukocyte dysfunctions, impairments of blood coagulation, graft-versus-host reaction; cancer such as, for example, breast carcinoma, colon tumour, gastrointestinal tumours, Hodgkin's lymphoma, non-Hodgkin's lymphoma, Kaposi sarcoma, liver tumour, pancreatic tumour, skin tumour, bone marrow tumour, leukaemias such as, for example, acute lymphatic leukaemia, acute myeloid leukaemia, chronic myeloid leukaemia, chronic lymphatic leukaemia, MLL leukaemia, prostate tumours, lung cancer, renal tumours; asthma, progressive, not completely reversible obstruction of the respiratory tract, pneumonia, pulmonary dysfunction; inflammatory disorders such as, for example, autoimmune diseases such as multiple sclerosis, rheumatoid arthritis, infections by gram-negative and gram-positive bacteria, viral infections, fungal infections such as, for example, by Candida albicans, HIV infections and HIV-associated infections, hepatitis of types A, B and C, parasitic infections; malaria; hair loss; reduced sperm motility; wound healing; osteoporosis, bone marrow disorders, bone and joint disorders; cardiovascular disorders such as, for example, cardiac defects, heart failure, cardiac fibrosis, cardiac arrhythmias, myocardial infarction, medicament- or substance-induced cardiotoxicity, atherosclerosis, high blood pressure; sepsis; inflammatory disorders.

The compounds according to the invention are particularly suitable for the prophylaxis and/or treatment of neurodegenerative disorders, such as, for example, Alzheimer's disease and schizophrenia, of type II diabetes mellitus and associated disorders, of cancer, of leukopenias and/or of neutropenias.

The compounds according to the invention are particularly suitable for the prophylaxis and/or treatment of leukopenias and/or of neutropenias.

The compounds according to the invention can additionally be employed also for efficient ex vivo expansion of adult haematopoietic stem cells from the bone marrow and/or from peripheral blood and/or for ex vivo expansion of embryonal stem cells from umbilical cord blood.

In addition, the compounds according to the invention can also be used for ex vivo expansion of embryonal and/or adult stem cells and also for ex vivo differentiation of embryonal and/or adult stem cells.

These cells expanded in this way can then be used to curtail the cytopenias induced by myeloablative therapies or within the framework of therapeutic transplantation methods or for haematological systemic disorders such as, for example, leukaemias, or with cells which have been genetically manipulated after expansion for gene therapies.

The present invention further relates to the use of the compounds according to the invention for the treatment and/or prophylaxis of disorders, especially of the aforementioned disorders.

The present invention further relates to the use of the compounds according to the invention for the manufacture of a medicament for the treatment and/or prophylaxis of disorders, especially of the aforementioned disorders.

The present invention further relates to a method for the treatment and/or prophylaxis of disorders, in particular of the aforementioned disorders, by use of a therapeutically effective amount of a compound according to the invention.

The present invention further relates to medicaments comprising a compound according to the invention and one or more further active ingredients, in particular for the treatment and/or prophylaxis of the aforementioned disorders. Suitable active ingredients in the combination which may be mentioned by way of example and preferably are:

A combination of the compounds according to the invention with chemotherapeutic agents used clinically may lead to a significantly improved result of treatment for various neoplastic diseases. The chemotherapeutic agents are substances which either inhibit the rate of division of tumour cells and/or prevent neovascularization of solid tumours. These include substances inter alia from the group of taxanes such as, for example, paclitaxel, or docetaxel, substances which inhibit the mitosis of tumour cells, such as, for example, vinblastine, vincristine, vindesine or vinorelbine. Substances from the class of platinum derivatives such as, for example, cisplatin, carboplatin, oxaliplatin, nedaplatin or lobaplatin. The chemotherapeutic agents further include substances from the class of alkylating agents such as, for example, cyclophosphamide, ifosfamide, melphalan, chlorambucil, pipobroman, triethylene melamine, busulphan, carmustine, lomustine, streptozin, dacarbazine or temozolomide. The chemotherapeutic agents also include antimetabolites such as, for example, folic acid antagonists, pyrimidine analogues, purine analogues or adenosine deaminase inhibitors. This class of substances includes inter alia methotrexate, 5-fluorouracil, floxuridine, cytarabine, pentostatin and gemcitabine. Also employed as chemotherapeutic agents are natural products or derivatives thereof, which include inter alia enzymes, antitumour antibodies and lymphokines. These include for example bleomycin, dactinomycin, daunorubicin, doxorubicin, epirubicin, idarubicin, ara-V, paclitaxel, mithramycin, mitomycin-C, L-asparaginase, interferons (e.g. IFN-alpha) and etoposide. Other chemotherapeutic agents with antiproliferative and/or anti-angiogenic effect are sorafenib, sunitinib, bortezomib, DAST inhibitor (BAY 73-4506), ZK epothilon inter alia.

The present invention further relates to a method for the ex vivo expansion of adult haematopoietic stem cells from bone marrow and/or from peripheral blood and/or for the ex vivo expansion of embryonal stem cells from umbilical cord blood, which is characterized in that an effective amount of the compound according to the invention is added.

The compounds of the invention can act systemically and/or locally. For this purpose, they can be administered in a suitable way such as, for example, by the oral, parenteral, pulmonary, nasal, sublingual, lingual, buccal, rectal, dermal, transdermal, conjunctival or otic route or as implant or stent.

The compounds of the invention can be administered in administration forms suitable for these administration routes.

Suitable for oral administration are administration forms which function according to the prior art and deliver the compounds of the invention rapidly and/or in modified fashion, and which contain the compounds of the invention in crystalline and/or amorphized and/or dissolved form, such as, for example, tablets (uncoated or coated tablets, for example having enteric coatings or coatings which are insoluble or dissolve with a delay and control the release of the compound of the invention), tablets which disintegrate rapidly in the mouth, or films/wafers, films/lyophilizates, capsules (for example hard or soft gelatin capsules), sugar-coated tablets, granules, pellets, powders, emulsions, suspensions, aerosols or solutions.

Parenteral administration can take place with avoidance of an absorption step (e.g. intravenous, intraarterial, intracardiac, intraspinal or intralumbar) or with inclusion of an absorption (e.g. intramuscular, subcutaneous, intracutaneous, percutaneous or intraperitoneal). Administration forms suitable for parenteral administration are, inter alia, preparations for injection and infusion in the form of solutions, suspensions, emulsions, lyophilizates or sterile powders.

Oral administration is preferred.

Suitable for the other administration routes are, for example, pharmaceutical forms for inhalation (inter alia powder inhalers, nebulizers), nasal drops, solutions, sprays; tablets for lingual, sublingual or buccal administration, films/wafers or capsules, suppositories, preparations for the ears or eyes, vaginal capsules, aqueous suspensions (lotions, shaking mixtures), lipophilic suspensions, ointments, creams, transdermal therapeutic systems (such as, for example, patches), milk, pastes, foams, dusting powders, implants or stents.

The compounds of the invention can be converted into the stated administration forms. This can take place in a manner known per se by mixing with inert, non-toxic, pharmaceutically suitable excipients. These excipients include, inter alia, carriers (for example microcrystalline cellulose, lactose, mannitol), solvents (e.g. liquid polyethylene glycols), emulsifiers and dispersants or wetting agents (for example sodium dodecyl sulphate, polyoxysorbitan oleate), binders (for example polyvinylpyrrolidone), synthetic and natural polymers (for example albumin), stabilizers (e.g. antioxidants such as, for example, ascorbic acid), colours (e.g. inorganic pigments such as, for example, iron oxides) and masking flavours and/or odours.

The present invention further relates to medicaments which comprise at least one compound of the invention, preferably together with one or more inert, non-toxic, pharmaceutically suitable excipients, and to the use thereof for the aforementioned purposes.

It has generally proved advantageous on parenteral administration to administer amounts of about 5 to 1500 mg every 24 hours to achieve effective results. The amount on oral administration is about 5 to 2000 mg every 24 hours.

It may nevertheless be necessary where appropriate to deviate from the stated amounts, in particular as a function of the body weight, route of administration, individual response to the active ingredient, nature of the preparation and time or interval over which administration takes place.

The percentage data in the following tests and examples are, unless indicated otherwise, percentages by weight; parts are parts by weight. Solvent ratios, dilution ratios and concentration data of liquid/liquid solutions are in each case based on volume. The statement “w/v” means “weight/volume”. Thus, for example, “10% w/v” means: 100 ml of solution or suspension comprise 10 g of substance.

### A) EXAMPLES

### Abbreviations

abs. absolute
Boc tert-butoxycarbonyl
CDCl₃ deuterochloroform
conc. concentrated
d day

### DIEA N,N-diisopropylethylamine

### DMAP 4-N,N-dimethylaminopyridine

DMF dimethylformamide
DMSO dimethyl sulphoxide
EDC N′-(3-dimethylaminopropyl)-N-ethylcarbodiimide×HCl
eq. equivalent
ESI electrospray ionization (in MS)
h hour
HOBt 1-hydroxy-1H-benzotriazole×H₂O
HPLC high pressure, high performance liquid chromatography
LC-MS coupled liquid chromatography-mass spectrometry
min. minutes
MS mass spectrometry
MW molecular weight [g/mol]
NMR nuclear magnetic resonance spectroscopy
OAc acetate
OEt ethoxy
p.a. per analysis
PyBOP 1-benzotriazolyloxytripyrrolidinophosphonium hexafluorophosphate
R_{f} retention index (in TLC)
sat. saturated
RP-HPLC reverse phase HPLC
RT room temperature
Rₜ retention time (in HPLC)
TBTU (benzotriazol-1-yloxy)bisdimethylaminomethylium fluoroborate
TFA trifluoroacetic acid
THF tetrahydrofuran

### LC-MS Methods:

Method 1: Instrument: Micromass Quattro LCZ with HPLC Agilent series 1100; column: Phenomenex Synergi 2.5μ MAX-RP 100A mercury 20 mm×4 mm; eluent A: 1 l of water+0.5 ml of 50% formic acid, eluent B: 1 l of acetonitrile+0.5 ml of 50% formic acid; gradient: 0.0 min 90% A→0.1 min 90% A→3.0 min 5% A→4.0 min 5% A→4.1 min 90% A; flow rate: 2 ml/min; oven: 50° C.; UV detection: 208-400 nm.

Method 2: MS instrument type: Micromass ZQ; HPLC instrument type: Waters Alliance 2795; column: Merck Chromolith SpeedROD RP-18e 100 mm×4.6 mm; eluent A: water+500 μl of 50% formic acid/l; eluent B: acetonitrile+500 μl of 50% formic acid/l; gradient: 0.0 min 10% B→7.0 min 95% B→9.0 min 95% B; oven: 35° C.; flow rate: 0.0 min 1.0 mL/min→7.0 min 2.0 mL/min→9.0 min 2.0 mL/min; UV detection: 210 nm

Method 3: MS instrument type: Micromass ZQ; HPLC instrument type: HP 1100 Series; UV DAD; column: Phenomenex Gemini 3μ 30 mm×3.00 mm; eluent A: 1 l of water+0.5 ml of 50% formic acid, eluent B: 1 l of acetonitrile+0.5 ml of 50% formic acid; gradient: 0.0 min 90% A→2.5 min 30% A→3.0 min 5% A→4.5 min 5% A; flow rate: 0.0 min 1 mL/min, 2.5 min/3.0 min/4.5 min. 2 ml/min; oven: 50° C.; UV detection: 210 nm.

Method 4: Instrument: Micromass Platform LCZ with HPLC Agilent series 1100; column: Thermo Hypersil GOLD 3μ 20 mm×4 mm; eluent A: 1 l of water+0.5 ml of 50% formic acid, eluent B: 1 l of acetonitrile+0.5 ml of 50% formic acid; gradient: 0.0 min 100% A→0.2 min 100% A→2.9 min 30% A→3.1 min 10% A→5.5 min 10% A; oven: 50° C.; flow rate: 0.8 mL/min; UV detection: 210 nm.

Method 5: MS instrument type: Waters ZQ; HPLC instrument type: Waters Alliance 2795; column: Phenomenex Onyx Monolithic C18, 100 mm×3 mm; eluent A: 1 l of water+0.5 ml of 50% formic acid, eluent B: 1 l of acetonitrile+0.5 ml of 50% formic acid; gradient: 0.0 min 90% A→2 min 65% A→4.5 min 5% A→6 min 5% A; flow rate: 2 mL/min; oven: 40° C.; UV detection: 210 nm.

Method 6: MS instrument type: Micromass ZQ; HPLC instrument type: Waters Alliance 2795; column: Phenomenex Synergi 2.5μ MAX-RP 100A mercury 20 mm×4 mm; eluent A: 1 l of water+0.5 ml of 50% formic acid, eluent B: 1 l of acetonitrile+0.5 ml of 50% formic acid; gradient: 0.0 min 90% A→0.1 min 90% A→3.0 min 5% A→4.0 min 5% A→4.01 min 90% A; flow rate: 2 ml/min; oven: 50° C.; UV detection: 210 nm.

Method 7: Instrument: Micromass Quattro LCZ with HPLC Agilent series 1100; column: Phenomenex Onyx Monolithic C18, 100 mm×3 mm; eluent A: 1 l of water+0.5 ml of 50% formic acid, eluent B: 1 l of acetonitrile+0.5 ml of 50% formic acid; gradient: 0.0 min 90% A→2 min 65% A→4.5 min 5% A→6 min 5% A; flow rate: 2 mL/min; oven: 40° C.; UV detection: 208-400 nm.

Method 8: Instrument: Micromass QuattroPremier with Waters HPLC Acquity; column: Thermo Hypersil GOLD 1.9μ 50 mm×1 mm; eluent A: 1 l of water+0.5 ml of 50% formic acid, eluent B: 1 l of acetonitrile+0.5 ml of 50% formic acid; gradient: 0.0 min 100% A→0.1 min 100% A→1.5 min 10% A→2.2 min 10% A; oven: 50° C.; flow rate: 0.33 ml/min; UV detection: 210 nm.

Method 9: Instrument: Micromass Quattro Micro MS with HPLC Agilent series 1100; column: Thermo Hypersil GOLD 3μ 20 mm×4 mm; eluent A: 1 l of water+0.5 ml of 50% formic acid, eluent B: 1 l of acetonitrile+0.5 ml of 50% formic acid; gradient: 0.0 min 100% A→3.0 min 10% A→4.0 min 10% A→4.01 min 100% A→5.00 min 100% A; flow rate: 0.0 min/3.0 min/4.0 min/4.01 min 2.5 mL/min, 5.00 min 2 ml/min; oven: 50° C.; UV detection: 210 nm.

Method 10: Preparative HPLC: column: Reprosil C18; gradient: acetonitrile/water with 0.1% hydrochloric acid.

Method 11: Preparative HPLC: column: Reprosil C18; gradient: acetonitrile/water with 0.1% trifluoroacetic acid.

Method 12: Instrument: Waters ACQUITY SQD UPLC System; column: Waters Acquity UPLC HSS T31.8μ, 50 mm×1 mm; eluent A: 1 l of water+0.25 ml of 99% strength formic acid, eluent B: 1 l of acetonitrile+0.25 ml of 99% strength formic acid; gradient: 0.0 min 90% A→1.2 min 5% A→2.0 min 5% A; flow rate: 0.40 ml/min; oven: 50° C.; UV detection: 210-400 nm.

Method 13: Preparative HPLC: column: Reprosil C18; gradient: acetonitrile/water.

The microwave reactor used was a “single mode” instrument of the Emrys™ Optimizer type.

### Starting Materials

### Example 1A

### tert-Butyl {2-[(5-cyanopyridin-2-yl)amino]ethyl}carbamate

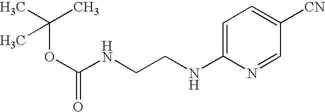

5.5 g (39.7 mmol) of 6-chloronicotinonitrile were dissolved in 70 ml of DMSO, and 10.2 g (63.5 mmol) of N-Boc-ethylenediamine and 11 g (79.4 mmol) of potassium carbonate were added. The mixture was stirred at 90° C. for 12 h. The residue was taken up in a mixture of water and ethyl acetate. The organic phase was washed with saturated aqueous sodium chloride solution, dried over magnesium sulphate and concentrated on a rotary evaporator. The residue was chromatographed on silica gel 60 (mobile phase: cyclohexane/ethyl acetate 10:1 to 2:1). This gave 7.9 g (77% of theory) of the product as a solid.

LCMS (method 6): Rₜ=1.46 min. (m/z=263 (M+H)⁺)

¹H-NMR (400 MHz, DMSO-d₆): δ=8.37 (d, 1H), 7.66 (d, 1H) 7.6 (s, 1H), 6.87 (t, 1H), 6.53 (d, 1H), 3.32 (q, 2H), 3.09 (q, 2H), 1.37 (s, 9H).

### Example 2A

### 6-[(2-Aminoethyl)amino]nicotinonitrile dihydrochloride

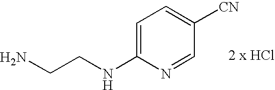

7.9 g (30 mmol) of tert-butyl {2-[(5-cyanopyridin-2-yl)amino]ethyl}carbamate (Example 1A) were dissolved in 100 ml 4N hydrogen chloride in dioxane and the mixture was stirred for 30 min. The reaction mixture was concentrated to half of its original volume, and the same volume of diethyl ether was added. The reaction mixture was stirred for 20 min, and the product was filtered off and washed with diethyl ether. This gave 7 g (94% of theory) of the product as a solid.

LCMS (method 4): Rₜ=0.51 min. (m/z=162 (M+H)⁺)

¹H-NMR (400 MHz, DMSO-d₆): δ=8.44 (s, 1H), 7.76 (d, 1H), 6.67 (d, 1H), 3.58 (t, 2H), 2.98 (q, 2H).

### Example 3A

### 2,4-Dichloro-N′-4H-1,2,4-triazol-4-ylbenzenecarboximidamide

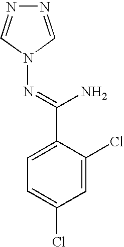

1.37 g (0.059 mol) of sodium were dissolved in 50 ml of ethanol, and 5 g (0.059 mol) of 4-amino-4H-1,2,4-triazole and 10.23 g (0.059 mol) of 2,6-dichlorobenzonitrile were added. The mixture was stirred under reflux for 5 h. After cooling, the solid obtained was filtered off and the filtrate was concentrated using a rotary evaporator. The solid was stirred in 120 ml of water under reflux for 15 min. The hot mixture was filtered with suction, and the solid was dried. The mother liquor was discarded. The filtrate was concentrated using a rotary evaporator, taken up in 150 ml of water, suspended in an ultrasonic bath and stirred under reflux for 15 min. The hot mixture was filtered off with suction, and the solid was dried. The mother liquor was discarded. Both solids were combined, giving 12.99 g (74% of theory) of the product.

LCMS (method 3): Rₜ=1.34 min. (m/z=256 (M+H)⁺).

¹H-NMR (400 MHz, DMSO-d₆): δ=8.44 (s, 2H), 7.76 (s, 1H), 7.59 (d, 1H), 7.55 (d, 1H).

### Example 4A

### N′-4H-1,2,4-Triazol-4-yl-4-(trifluoromethyl)benzenecarboximidamide

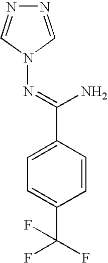

The compound was prepared analogously to Example 3A. The starting material used was 4-(trifluoromethyl)benzonitrile instead of 2,6-dichlorobenzonitrile.

LCMS (method 3): Rₜ=1.55 min. (m/z=256 (M+H)⁺).

¹H-NMR (400 MHz, DMSO-d₆): δ=8.5 (s, 2H), 8.11 (d, 2H), 7.87 (d, 2H), 7.56 (br, s, 2H).

### Example 5A

### Butyl [(Z)-(2,4-dichlorophenyl)(4H-1,2,4-triazol-4-ylimino)methyl]carbamate

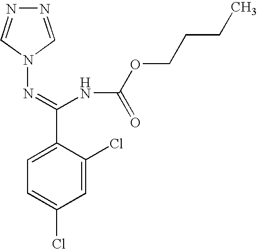

808 mg (35.1 mmol) of sodium were dissolved in 50 ml of 1-butanol, and 6 g (23.4 mmol) of 2,4-dichloro-N′-4H-1,2,4-triazol-4-ylbenzenecarboximidamide (Example 3A), and 6.2 ml (51.5 mmol) of diethyl carbonate were added. The mixture was stirred under reflux for 2 h. After cooling, ethyl acetate and water were added, and the pH was adjusted to 5 using concentrated hydrochloric acid. The phases were separated, and the organic phase was dried over magnesium sulphate and concentrated on a rotary evaporator. The reaction mixture was chromatographed on silica gel (mobile phase dichloromethane/methanol 100:1→50:1). This gave 4.8 g (56% of theory) of the product as a solid.

LCMS (method 8): Rₜ=1.08 min. (m/z=356 (M+H)⁺).

### Example 6A

### Butyl {(Z)-(4H-1,2,4-triazol-4-ylimino)[4-(trifluoromethyl)phenyl]methyl}carbamate

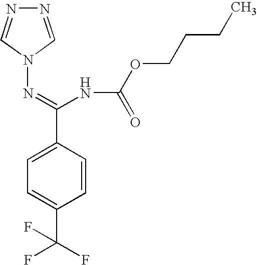

The compound was prepared analogously to Example 5A from N′-4H-1,2,4-triazol-4-yl-4-(trifluoromethyl)benzenecarboximidamide (Example 4A).

LCMS (method 6): Rₜ=1.73 min. (m/z=356 (M+H)⁺).

¹H-NMR (400 MHz, DMSO-d₆): δ=10.75 (s, 1H), 8.8 (s, 2H), 7.91 (d, 2H), 7.87 (d, 2H), 3.97 (t, 2H), 1.45 (q, 2H), 1.2 (m, 2H), 0.83 (t, 3H).

### Example 7A

### 6-(2,4-Dichlorophenyl)[1,2,4]triazolo[3,4-f][1,2,4]triazin-8(7H)-one

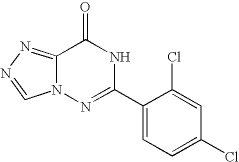

4.7 g (13.4 mmol) of butyl [(Z)-(2,4-dichlorophenyl)(4H-1,2,4-triazol-4-ylimino)methyl]carbamate (Example 5A) in 25 ml of phenol were stirred under reflux for 5 h. The reaction mixture was diluted with dichloromethane. The reaction mixture was chromatographed on silica gel (mobile phase dichloromethane/methanol 100%→50:1). This gave 3.1 g (78% of theory) of the product as a solid.

LCMS (method 3): Rₜ=1.62 min. (m/z=282 (M+H)⁺).

¹H-NMR (400 MHz, DMSO-d₆): δ=13.0 (s, 1H), 9.5 (s, 1H), 7.99 (ss, 1H), 7.71 (d, 1H), 7.65 (dd, H).

### Example 8A

### 6-[4-(Trifluoromethyl)phenyl][1,2,4]triazolo[3,4-f][1,2,4]triazin-8(7H)-one

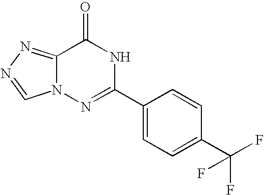

The compound was prepared analogously to Example 7A from butyl {(Z)-(4H-1,2,4-triazol-4-ylimino)[4-(trifluoromethyl)phenyl]methyl}carbamate (Example 6A).

LCMS (method 9): Rₜ=0.87 min. (m/z=282 (M+H)⁺).

¹H-NMR (400 MHz, DMSO-d₆): δ=13.05 (br, s, 1H), 9.52 (s, 1H), 8.16 (d, 2H), 7.97 (d, 2H).

### Example 9A

### 8-Chloro-6-(2,4-dichlorophenyl)[1,2,4]triazolo[3,4-f][1,2,4]triazine

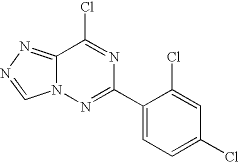

4.7 g (13.4 mmol) of 6-(2,4-dichlorophenyl)[1,2,4]triazolo[3,4-f][1,2,4]triazin-8(7H)-one (Example 7A) were initially charged in 20 ml of phosphoryl chloride, and 4.8 g (21.7 mmol) of benzyltriethylammonium chloride were added. The mixture was stirred at 120° C. for 2 h. The reaction mixture was poured into 150 ml of saturated sodium bicarbonate solution, and solid sodium bicarbonate was added until a pH of 7 had been reached. The precipitated solid was filtered off with suction and dried. This gave 1.3 g (75% of theory) of the product as a solid.

LCMS (method 3): Rₜ=2.23 min. (m/z=300 (M+H)⁺).

¹H-NMR (400 MHz, DMSO-d₆): δ=9.51 (s, 1H), 7.9 (ss, 1H), 7.71 (d, 1H), 7.66 (dd, 1H).

### Example 10A

### 8-Chloro-6-[4-(trifluoromethyl)phenyl][1,2,4]triazolo[3,4-f][1,2,4]triazine

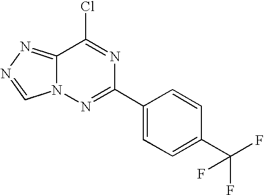

The compound was prepared analogously to Example 9A from 6-[4-(trifluoromethyl)-phenyl][1,2,4]triazolo[3,4-f][1,2,4]triazin-8(7H)-one (Example 8A).

LCMS (method 3): Rₜ=2.3 min. (m/z=300 (M+H)⁺).

¹H-NMR (400 MHz, DMSO-d₆): δ=9.52 (s, 1H), 8.16 (d, 1H), 7.97 (d, 1H).

### Example 11A

### tert-Butyl (6-chloropyridin-2-yl)carbamate

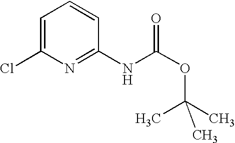

Under argon, 150 ml of THF were added to 23.4 g (181.8 mmol) of 2-chloro-5-aminopyridine, and the mixture was cooled to 0° C. 73.3 g (400 mmol) of sodium bis(trimethylsilyl)amide and 43.65 g (200 mmol) of di-tert-butyl dicarbonate, dissolved in 150 ml of THF, were added dropwise. After 15 min, the cooling bath was removed, and stirring was continued at RT for 15 min. The THF was removed using a rotary evaporator, ethyl acetate and 0.5 N hydrochloric acid were added to the residue and the mixture was extracted. The organic phase was separated off, dried over magnesium sulphate and concentrated using a rotary evaporator. The reaction mixture was chromatographed on silica gel (mobile phase dichloromethane/methanol 100%→100:3). This gave 36.54 g (88% of theory) of the product as a solid.

LCMS (method 3): Rₜ=2.41 min. (m/z=175 (M+H)⁺).

¹H-NMR (400 MHz, DMSO-d₆): δ=10.11 (s, 1H), 7.78 (d, 2H), 7.1 (t, 1H), 1.47 (s, 9H).

### Example 12A

### tert-Butyl (6-chloro-3-formylpyridin-2-yl)carbamate

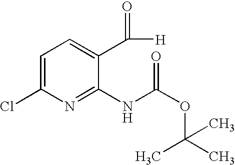

The reaction apparatus was dried by heating, and the reaction was carried out under argon and with stirring. 15 g (65.6 mmol) of tert-butyl (6-chloropyridin-2-yl)carbamate (Example 11A) and 19 g (164 mmol) of 1,2-bis(dimethylamino)ethane were initially charged in 270 ml of THF and cooled to −78° C. 102.5 ml (164 mmol) of butyllithium (1.6N) were added dropwise. After the dropwise addition had ended, the reaction was slowly warmed to −10° C. and kept at −10° C. for 2 h. The mixture was then once more cooled to −78° C., and 10 ml (131 mmol) of DMF were added. The reaction was slowly warmed to RT, the reaction mixture was then added to 1 l of ethyl acetate and 350 ml of 1N hydrochloric acid and stirred for 15 min and the organic phase was separated off. The organic phase was washed with water and saturated sodium bicarbonate solution, dried over magnesium sulphate and concentrated on a rotary evaporator. Diethyl ether was added to the residue, and the solid was filtered off with suction and then dried. This gave 12.3 g (73% of theory) of the product as a solid.

LCMS (method 3): Rₜ=2.19 min. (m/z=255 (M+H)⁻).

¹H-NMR (400 MHz, DMSO-d₆): δ=10.37 (s, 1H), 9.83 (s, 1H), 8.2 (d, 1H), 7.42 (d, 1H), 1.46 (s, 9H).

### Example 13A

### tert-Butyl {6-chloro-3-[(hydroxyimino)methyl]pyridin-2-yl}carbamate

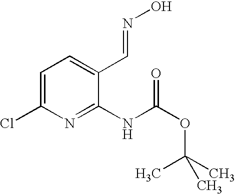

15.45 g (60.2 mmol) of tert-butyl (6-chloro-3-formylpyridin-2-yl)carbamate (Example 12A) were initially charged in 750 ml of ethanol, a solution of 225 ml of water and 9.38 g (120.4 mmol) of sodium acetate was added and the mixture was stirred for 5 min. A solution of 225 ml of water and 8.36 g (114.4 mmol) of hydroxylamine hydrochloride was added, and the mixture was stirred at RT for 4 h. At 20° C., the reaction mixture was concentrated on a rotary evaporator. The residue was taken up in ethyl acetate and washed twice with saturated sodium bicarbonate solution and once with saturated sodium chloride solution. The organic phase was separated off, dried over magnesium sulphate and dried at 20° C. on a rotary evaporator. This gave 15.5 g (80% of theory) of the product as a solid.

LCMS (method 3): Rₜ=2.08 min. (m/z=270 (M+H)⁻).

¹H-NMR (400 MHz, DMSO-d₆): δ=11.71 (s, 1H), 9.91 (s, 1H), 8.14 (s, 1H), 8.02 (d, 1H), 7.3 (d, 1H), 1.49 (s, 9H).

### Example 14A

### 2-Amino-6-chloropyridine-3-carbaldehyde oxime hydrochloride

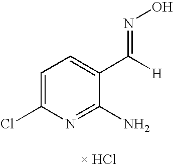

15.5 g (57 mmol) of tert-butyl {6-chloro-3-[(hydroxyimino)methyl]pyridin-2-yl}carbamate (Example 13A) were dissolved in 285 ml of 4N hydrogen chloride in dioxane, and the mixture was stirred for 30 min. The reaction mixture was concentrated to half of its original volume, and the same volume of diethyl ether was added. The reaction mixture was stirred for 20 min, and the product was filtered off and washed with diethyl ether. This gave 11 g (94% of theory) of the product as a solid.

LCMS (method 6): Rₜ=1.09 min. (m/z=172 (M+H)⁺)

¹H-NMR (400 MHz, DMSO-d₆): δ=8.27 (s, 1H), 7.61 (d, 1H), 6.65 (d, 1H).

### Example 15A

### 2-Amino-6-chloropyridine-3-carbonitrile

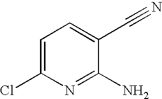

11.15 g (53.6 mmol) of 2-amino-6-chloropyridine-3-carbaldehyde oxime hydrochloride (Example 14A) were initially charged in dioxane, 13 ml (161 mmol) of pyridine were added and the mixture was cooled to 0° C. 8.3 ml (58.95 mmol) of trifluoroacetic anhydride were added, and the reaction was warmed to RT and then stirred at 60° C. for 2 h. The reaction mixture was taken up in a mixture of ethyl acetate and sodium bicarbonate solution. The organic phase was washed with saturated aqueous sodium chloride solution, dried over magnesium sulphate and concentrated on a rotary evaporator. The residue was suspended in dichloromethane:diethyl ether 3:1, and the solid was filtered off with suction and dried. This gave 5.56 g (66% of theory) of the product as a solid.

LCMS (method 6): Rₜ=1.0 min. (m/z=154 (M+H)⁺).

¹H-NMR (400 MHz, DMSO-d₆): δ=7.91 (d, 1H), 7.38 (s, 2H), 6.69 (d, 1H).

### Example 16A

### tert-Butyl {2-[(6-amino-5-cyanopyridin-2-yl)amino]ethyl}carbamate

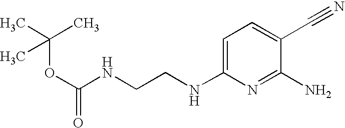

2 g (13 mmol) of 2-amino-6-chloropyridine-3-carbonitrile (Example 15A) were initially charged in 15 ml of DMSO, and 2.71 g (16.93 mmol) of N-Boc-ethyleneamine and 3.4 ml (19.54 mmol) of N,N-diisopropylethylamine were added. At 115° C., the reaction mixture was irradiated in the microwave reactor for 1.5 h. The reaction mixture was taken up in a mixture of ethyl acetate and water. The organic phase was washed with saturated aqueous sodium chloride solution, dried over magnesium sulphate and concentrated on a rotary evaporator. This gave 23.38 g (88% of theory) of the product as a solid.

LCMS (method 3): Rₜ=1.7 min. (m/z=278 (M+H)⁺).

¹H-NMR (400 MHz, DMSO-d₆): δ=7.3 (s, 1H), 7.0 (br, s, 1H), 6.83 (s, 1H), 6.25 (s, 2H), 5.78 (d, 1H), 3.25 (q, 2H), 3.06 (q, 2H), 1.36 (s, 9H).

### Example 17A

### 2-Amino-6-[(2-aminoethyl)amino]pyridine-3-carbonitrile dihydrochloride

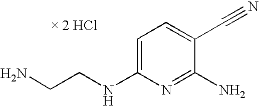

6.76 g (24.38 mmol) of tert-butyl {2-[(6-amino-5-cyanopyridin-2-yl)amino]ethyl}carbamate (Example 16A) were dissolved in 122 ml of 4N hydrogen chloride solution in dioxane and stirred for 30 min. The reaction mixture was concentrated to half of its original volume, and the same volume of diethyl ether was added. The reaction mixture was stirred for 20 min, and the product was filtered off and washed with diethyl ether. This gave 5.43 g (89% of theory) of the product as a solid.

LCMS (method 6): Rₜ=0.92 min. (m/z=177 (M+H)⁺)

¹H-NMR (400 MHz, DMSO-d₆): δ=8.1 (s, 2H), 7.5 (d, 1H), 5.96 (d, 1H), 3.53 (q, 2H), 3.0 (q, 2H).

### Example 18A

### Methyl 1-[2-(2,4-dichlorophenyl)-2-oxoethyl]-1H-1,2,4-triazole-5-carboxylate

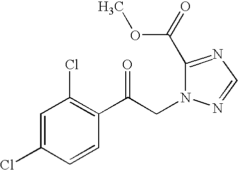

4.3 g (33.8 mmol) of methyl 1H-1,2,4-triazole-3-carboxylate were initially charged in 58 ml of acetone, and 7.9 g (35.4 mmol) of 2′-chloro-2,4-dichloracetophenone and 5.3 g (38.9 mmol) of potassium carbonate were added. The mixture was stirred at RT for 20 h. The reaction mixture was then concentrated at 20° C. on a rotary evaporator. The residue was taken up in dichloromethane and washed twice with water and once with saturated sodium chloride solution. The organic phase was separated off, dried over magnesium sulphate and concentrated at 20° C. on a rotary evaporator. The reaction mixture was chromatographed on silica gel (mobile phase dichloromethane/ethanol 100:1). This gave 1.47 g (10% of theory) of the product as a solid.

LCMS (method 8): Rₜ=1.08 min. (m/z=314 (M+H)⁺)

¹H-NMR (400 MHz, DMSO-d₆): δ=8.27 (s, 1H), 8.00 (d, 1H), 7.85 (ss, 1H), 7.68 (dd, 1H), 6.12 (s, 2H), 3.85 (s, 3H).

### Example 19A

### 6-(2,4-Dichlorophenyl) [1,2,4]triazolo[1,5-a]pyrazin-8(7H)-one

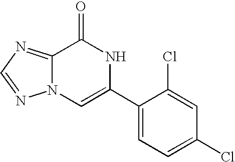

In glacial acetic acid, 1.44 g (3.5 mmol) of methyl 1-[2-(2,4-dichlorophenyl)-2-oxoethyl]-1H-1,2,4-triazole-5-carboxylate (Example 18A) and 2.7 g (35 mmol) of ammonium acetate were stirred under reflux for 24 h. The reaction mixture was cooled and added to ice-water. The pH was adjusted to 4 using sodium bicarbonate, and the precipitated solid was filtered off with suction and dried. This gave 460 mg (47% of theory) of the product as a solid.

LCMS (method 6): Rₜ=1.32 min. (m/z=281 (M+H)⁺)

¹H-NMR (400 MHz, DMSO-d₆): δ=12.23 (s, 1H), 8.53 (s, 1H), 8.09 (s, 1H), 7.84 (ss, 1H), 7.64 (d, 1H), 7.59 (dd, 1H).

### Example 20A

### 8-Chloro-6-(2,4-dichlorophenyl) [1,2,4]triazolo[1,5-a]pyrazine

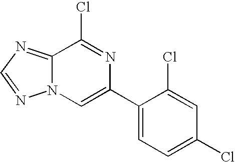

460 mg (1.6 mmol) of 6-(2,4-dichlorophenyl)[1,2,4]triazolo[1,5-a]pyrazin-8(7H)-one (Example 19A) were initially charged in 18 ml of phosphoryl chloride, and 1.1 g (4.9 mmol) of benzyltriethylammonium chloride were added. The mixture was stirred at 120° C. for 2 h. The reaction mixture was poured into 150 ml of saturated sodium bicarbonate solution, and solid sodium bicarbonate was added until a pH of 7 had been reached. The precipitated solid was filtered off with suction and dried. This gave 360 mg (73% of theory) of the product as a solid.

LCMS (method 6): Rₜ=1.97 min. (m/z=299 (M+H)⁺)

¹H-NMR (400 MHz, DMSO-d₆): δ=8.5 (s, 1H), 8.89 (s, 1H), 7.85 (ss, 1H), 7.72 (d, 1H), 7.63 (dd, 1H).

### Example 21A

### 4-(Trifluoroacetyl)morpholine

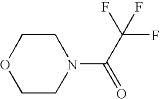

15 g (172 mmol) of morpholine were initially charged in 750 ml of dichloromethane, and 29 ml (206 mmol) of trifluoroacetic anhydride and 119 ml (688 mmol) of N,N-diisopropylethylamine were added at 0° C. The reaction mixture was warmed to RT and stirred at RT for 3 h. The reaction mixture was concentrated and the residue was taken up in ethyl acetate and washed successively with aqueous sodium bicarbonate solution, 1N hydrochloric acid and again with aqueous sodium bicarbonate solution. The organic phase was dried over magnesium sulphate and concentrated on a rotary evaporator. This gave 28 g (88% of theory) of the product as an oil.

LCMS (method 9): Rₜ=1.22 min. (m/z=184 (M+H)⁺)

¹H-NMR (400 MHz, DMSO-d₆): δ=3.65 (m, 2H), 3.56 (m, 2H).

### Example 22A

### tert-Butyl [6-chloro-3-(trifluoroacetyl)pyridin-2-yl]carbamate

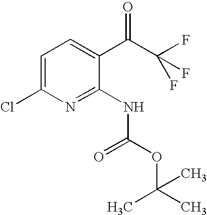

8 g (35 mmol) of tert-butyl (6-chloropyridin-2-yl)carbamate (Example 11A) were initially charged in 100 ml of THF and cooled to −50° C. 55 ml (87 mmol) of butyllithium (1.6N) were added dropwise. After the dropwise addition had ended, the reaction was slowly warmed to −10° C. and stirred at 0° C. for 2 h. The mixture was then once more cooled to −40° C., and 12.8 g (70 mmol) of 4-(trifluoroacetyl)morpholine (Example 21A), dissolved in 4 ml THF, were added. The reaction solution was stirred at −40° C. for 1 h and then, at −40° C., poured into 1 l of ethyl acetate and 350 ml of ammonium chloride solution and extracted. The organic phase was separated off, dried over magnesium sulphate and concentrated on a rotary evaporator. The reaction mixture was chromatographed on silica gel (mobile phase cyclohexane/ethyl acetate 10:1). This gave 9 g (79% of theory) of the product as an oil.

¹H-NMR (400 MHz, DMSO-d₆): δ=10.96 (s, 1H), 7.99 (d, 1H), 7.4 (d, 1H), 1.43 (s, 9H).

### Example 23A

### tert-Butyl [6-({2-[(tert-butoxycarbonyl)amino]ethyl}amino)-3-(trifluoroacetyl)pyridin-2-yl]carbamate

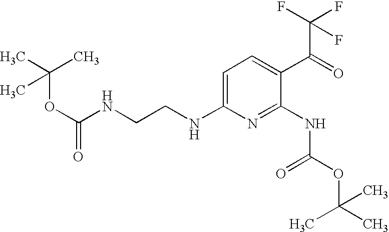

5 g (15.4 mmol) of tert-butyl [6-chloro-3-(trifluoroacetyl)pyridin-2-yl]carbamate (Example 22A) were initially charged in 37.5 ml of DMSO, and 3.2 g (20 mmol) of N-Boc-ethylenediamine and 4 ml (23 mmol) of N,N-diisopropylethylamine were added. The reaction mixture was, at 90° C., irradiated in the microwave reactor for 0.5 h. The reaction mixture was taken up in a mixture of ethyl acetate and water. The organic phase was washed with saturated aqueous sodium chloride solution, dried over magnesium sulphate and concentrated on a rotary evaporator. The reaction mixture was chromatographed on silica gel (mobile phase cyclohexane/ethyl acetate 5:1→1:1). This gave 2.5 g (34% of theory) of the product as a solid.

LCMS (method 6): Rₜ=2.44 min. (m/z=449 (M+H)⁺).

¹H-NMR (400 MHz, DMSO-d₆): δ=10.75 (s, 1H), 8.44 (s, 1H), 7.70 (d, 1H), 6.77 (s, 1H), 6.28 (d, 1H), 3.48 (br, s, 2H), 3.17 (br, s, 2H), 1.46 (s, 9H), 1.30 (s, 9H).

### Example 24A

### 1-{2-Amino-6-[(2-aminoethyl)amino]pyridin-3-yl}-2,2,2-trifluoroethanone hydrochloride

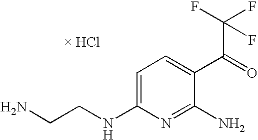

2.5 g (5.57 mmol) of tert-butyl [6-({2-[(tert-butoxycarbonyl)amino]ethyl}amino)-3-(trifluoroacetyl)pyridin-2-yl]carbamate (Example 23A) were dissolved in 15 ml of a 4N solution of hydrogen chloride in dioxane, and the mixture was stirred for 20 h. The reaction mixture was concentrated to half of its original volume, and the same volume of diethyl ether was added. The reaction mixture was stirred for 20 min, and the product was filtered off and washed with diethyl ether. This gave 1.4 g (89% of theory) of the product as a solid.

LCMS (method 6): Rₜ=0.73 min. (m/z=249 (M+H)⁺).

### Example 25A

### 4-Amino-2-(methylsulphonyl)-1,3-thiazole-5-carbonitrile

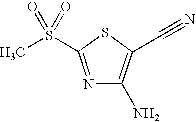

2.7 g (15.77 mmol) of 4-amino-2-(methylthio)-1,3-thiazole-5-carbonitrile were dissolved in 200 ml of dichloromethane, and 11.97 g (34.7 mmol) of 3-chloroperbenzoic acid were added. The mixture was stirred at RT for 30 min, 6 ml of DMSO were then added, followed by saturated aqueous sodium bicarbonate solution, and the mixture was extracted three times with dichloromethane. The organic phase was dried over magnesium sulphate giving, after removal of the solvent, 2.22 g (46% of theory) of the product as an oil which was used without further purification.

LCMS (method 3): Rₜ=1.19 min. (m/z=204 (M+H)⁺).

### Example 26A

### tert-Butyl {2-[(4-amino-5-cyano-1,3-thiazol-2-yl)amino]ethyl}carbamate

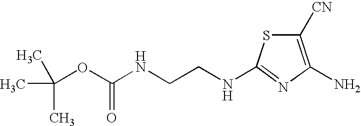

2.2 g (7.22 mmol) of 4-amino-2-(methylsulphonyl)-1,3-thiazole-5-carbonitrile (Example 18A) were dissolved in 24 ml of DMSO, and 1.74 g (10.84 mmol) of N-Boc-ethylenediamine and 933 mg (7.22 mmol) of DIEA were added. The mixture was stirred at 120° C. for 16 h and, after the reaction had ended, water and ethyl acetate were added. The aqueous phase was extracted three times with ethyl acetate. The combined organic phases were dried over magnesium sulphate and purified by silica gel chromatography. This gave 633 mg (31% of theory) of the product.

LCMS (method 6): Rₜ=1.45 min. (m/z=284 (M+H)⁺).

¹H-NMR (400 MHz, DMSO-d₆): δ=8.35 (s, br, 1H), 6.90 (t, 1H), 6.68 (s, 2H), 3.22 (s, br, 2H), 3.07 (dd, 2H), 1.37 (s, 9H).

### Example 27A

### 4-Amino-2-[(2-aminoethyl)amino]-1,3-thiazole-5-carbonitrile trifluoroacetate

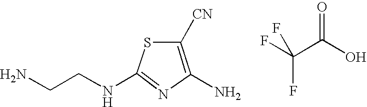

Analogously to the preparation of the cyanopyridine (Example 2A), 130 mg (0.46 mmol) of the Boc-protected amine (Example 19A) and 1.05 g (9.18 mmol) of trifluoroacetic acid gave, after removal of all volatile components, 130 mg (96% of theory) of the product.

LCMS (method 4): Rₜ=0.61 min. (m/z=184 (M+H)⁺).

¹H-NMR (400 MHz, DMSO-d₆): δ=8.45 (t, 1H), 7.84 (s, br, 2H), 6.80 (s, br, 1H), 3.93 (s, 1H), 3.43 (dd, 2H), 3.01 (dd, 2H).

### Example 28A

### tert-Butyl-3-[(5-cyanopyridin-2-yl)amino]piperidine-1-carboxylate

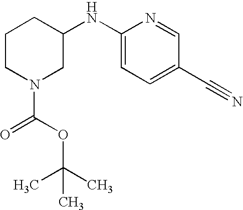

1.0 g (4.99 mmol) of tert-butyl 3-aminopiperidine-1-carboxylate and 1.383 g (9.99 mmol) of 6-chloropyridine-3-carbonitrile and 1.29 g (9.99 mmol) of diisopropylethylamine were suspended in 40 ml of DMSO and heated at 140° C. in a microwave reactor for 45 min. Most of the DMSO of the mixture was removed by kugelrohr distillation, water was added to the residue and the resulting precipitate was filtered off. Drying under high vacuum gave 2.24 g (46% of theory) of the product.

LCMS (method 3): Rₜ=2.23 min. (m/z=303 (M+H)⁺).

### Example 29A

### tert-Butyl 3-[(6-amino-5-cyanopyridin-2-yl)amino]piperidine-1-carboxylate

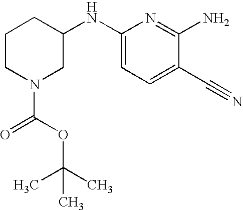

2.15 g (10.7 mmol) of tert-butyl 3-aminopiperidine-1-carboxylate, 1.50 g (9.77 mmol) of 2-amino-6-chloropyridine-3-carbonitrile (Example 15A) and 1.89 g (14.7 mmol) of diisopropylethylamine were suspended in 6 ml of DMSO and heated in a microwave reactor at 130° C. for 8 h. The reaction mixture was diluted with ethyl acetate (100 ml) and water (40 ml), and the organic phase was separated off, washed with saturated aqueous sodium chloride solution (50 ml), dried over magnesium sulphate and concentrated. The residue was chromatographed on silica gel (mobile phase: cyclohexane/ethyl acetate 4:1 to 1:1). 2.04 g (60% of theory) of the product were isolated as a solid.

LCMS (method 6): Rₜ=1.69 min. (m/z=318 (M+H)⁺)

### Example 30A

### 6-(Piperidin-3-ylamino)pyridine-3-carbonitrile hydrochloride

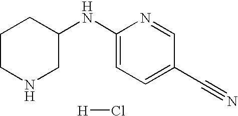

2.24 g (7.4 mmol) of tert-butyl 3-[(5-cyanopyridin-2-yl)amino]piperidine-1-carboxylate (Example 28A) were dissolved in 4.3 ml of a solution of hydrochloric acid in dioxane (4 M), and the mixture was stirred at RT for 3 h. After the reaction had gone to completion, the solvent was removed completely. This gave 1.74 g (90% of theory) of the product as a solid.

LCMS (method 8): Rₜ=0.27 min. (m/z=203 (M+H)⁺)

¹H-NMR (400 MHz, DMSO-d₆): δ=9.13 (m, 1H), 9.0 (m, 1H), 8.44 (d, 1H), 7.89 (m, 1H), 7.74 (dd, 1H), 6.63 (d, 1H), 5.58 (s, br), 4.19 (s, br, 1H), 3.57 (s, 1H), 3.34 (d, 1H), 3.14 (d, 1H), 2.88 (m, 1H), 2.7-2.81 (m, 1H), 1.82-2.0 (m, 2H), 1.63-1.79 (m, 1H), 1.48-1.59 (m, 1H).

### Example 31A

### 2-Amino-6-(piperidin-3-ylamino)pyridine-3-carbonitrile hydrochloride

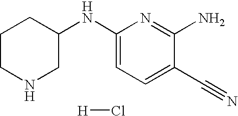

2.00 g (6.3 mmol) of tert-butyl 3-[(6-amino-5-cyanopyridin-2-yl)amino]piperidine-1-carboxylate (Example 29A) were dissolved in 40 ml of a solution of hydrochloric acid in dioxane (4 M), and the mixture was stirred at RT for 2 h. After the reaction had gone to completion, the solvent was concentrated to half of its original volume, and 20 ml of diethyl ether were added. The precipitate was filtered off and dried. This gave 1.80 g (100% of theory) of the product as a solid.

LCMS (method 8): Rₜ=0.25 min. (m/z=218 (M+H)⁺)

¹H-NMR (400 MHz, DMSO-d₆): δ=9.38 (br m, 1H), 8.97 (br m, 1H), 8.25 (br m, 1H), 7.53 (m, 1H), 7.40 (br s, 2H), 6.01 (d, 1H), 4.16 (br m, 1H), 3.34 (br m, 1H), 3.10 (m, 1H), 2.89 (m, 2H), 2.00-1.84 (m, 2H), 1.73 (m, 1H), 1.55 (m, 1H).

### Example 32A

### tert-Butyl 3-({6-[(tert-butoxycarbonyl)amino]-5-(trifluoroacetyl)pyridin-2-yl}-amino)piperidine-1-carboxylate

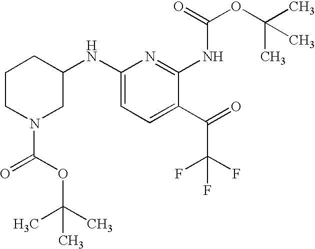

561 mg (2.8 mmol) of tert-butyl 3-aminopiperidine-1-carboxylate, 700 mg (2.16 mmol) of tert-butyl [6-chloro-3-(trifluoroacetyl)pyridin-2-yl]carbamate (Example 22A) and 0.56 ml (3.23 mmol) of diisopropylethylamine were suspended in 14 ml of DMSO, and the mixture was heated in a microwave reactor at 90° C. for 45 min. The reaction mixture was diluted with ethyl acetate (100 ml) and washed with saturated aqueous ammonium chloride solution (3×40 ml) and then with saturated aqueous sodium bicarbonate solution (40 ml). The organic phase was dried over magnesium sulphate and concentrated. The residue was chromatographed on silica gel (mobile phase: cyclohexane/ethyl acetate 5:1 to 1:1). 670 mg (63% of theory) of the product were isolated.

LCMS (method 6): Rₜ=2.70 min. (m/z=489 (M+H)⁺)

### Example 33A

### 1-[2-Amino-6-(piperidin-3-ylamino)pyridin-3-yl]-2,2,2-trifluoroethanone hydrochloride

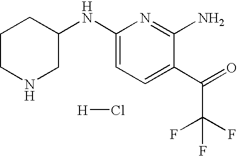

670 mg (1.37 mmol) of tert-butyl 3-({6-[(tert-butoxycarbonyl)amino]-5-(trifluoroacetyl)pyridin-2-yl}amino)piperidine-1-carboxylate (Example 32A) were dissolved in 25 ml of a solution of hydrochloric acid in dioxane (4 M), and the mixture was stirred at RT for 20 h. After reaction had gone to completion, the reaction mixture was diluted with diethyl ether (100 ml), and the precipitate was filtered off, washed with diethyl ether (100 ml) and dried. This gave 286 mg (64% of theory) of the product as a solid.

LCMS (method 6): Rₜ=0.81 min. (m/z=289 (M+H)⁺)

¹H-NMR (400 MHz, DMSO-d₆): δ=9.26 (br s, 1H), 9.07 (br s, 1H), 8.8.34 (br s, 1H), 7.59 (d, 1H), 6.22 (br, 2H), 6.03 (d, 1H), 4.25 (br m, 1H), 3.36 (m, 1H), 3.13 (m, 1H), 2.93 (m, 2H), 2.00-1.85 (m, 2H), 1.73 (m, 1H), 1.56 (m, 1H).

### Example 34A

### tert-Butyl (6-chloro-3-propanoylpyridin-2-yl)carbamate

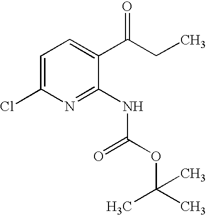

Under argon, 7.00 g (30.6 mmol) of tert-butyl (6-chloropyridin-2-yl)carbamate (Example 11A) were initially charged in 90 ml of THF and cooled to −50° C. 47.8 ml (76.5 mmol) of butyllithium (1.6 M in hexane) were added dropwise. After the dropwise addition had ended, the reaction was slowly warmed to 0° C. and kept at 0° C. for 1 h. The mixture was then again cooled to −40° C., and 9.85 g (61.2 mmol) of N-propionylmorpholine, dissolved in 10 ml of THF, were added. The reaction solution was stirred at −40° C. for 1 h and then, at −40° C., put into 1 l of ethyl acetate and 350 ml of ammonium chloride solution, and the organic phase was separated off and washed with water and saturated aqueous sodium bicarbonate solution. The organic phase was dried over magnesium sulphate and concentrated on a rotary evaporator. The crude product was chromatographed on silica gel (mobile phase cyclohexane/ethyl acetate 10:1 to 1:1). This gave 2800 mg (32% of theory) of the product.

LCMS (method 6): Rₜ=2.13 min. (m/z=283 (M−H)⁻)

¹H-NMR (400 MHz, DMSO-d₆): δ=10.53 (br s, 1H), 8.19 (d, 1H), 7.31 (d, 1H), 2.94 (q, 2H), 1.45 (s, 9H), 1.06 (t, 3H).

### Example 35A

### tert-Butyl [6-({2-[(tert-butoxycarbonyl)amino]ethyl}amino)-3-propanoylpyridin-2-yl]carbamate

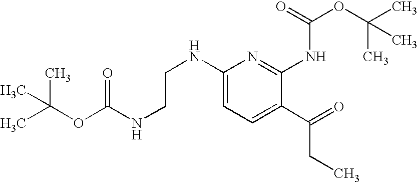

730 mg (2.4 mmol) of tert-butyl (6-chloro-3-propanoylpyridin-2-yl)carbamate (Example 34A) were initially charged in 7 ml of DMSO, and 512 mg (3.2 mmol) of N-Boc-ethylenediamine and 640 μl (3.7 mmol) of N,N-diisopropylethylamine were added. The reaction mixture was irradiated in the microwave reactor at 90° C. for 45 min. The reaction mixture was taken up in a mixture of ethyl acetate and water. The organic phase was washed with saturated aqueous ammonium chloride solution and then with saturated aqueous bicarbonate solution, dried over magnesium sulphate and concentrated on a rotary evaporator. The reaction mixture was chromatographed on silica gel (mobile phase cyclohexane/ethyl acetate 5:1→1:1). This gave 530 mg (53% of theory) of the product as a solid.

LCMS (method 6): Rₜ=2.19 min. (m/z=409 (M+H)⁺).

¹H-NMR (400 MHz, DMSO-d₆): δ=11.47 (s, 1H), 7.93 (br m, 1H), 7.64 (br m, 1H), 6.82 (br s, 1H), 6.15 (d, 1H), 3.43 (br m, 2H), 3.14 (m, 2H), 2.83 (q, 2H), 2.56 (br s, 4H), 1.47 (s, 9H), 1.32 (s, 9H), 1.03 (t, 3H).

### Example 36A

### 1-{2-Amino-6-[(2-aminoethyl)amino]pyridin-3-yl}propan-1-one hydrochloride

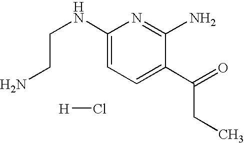

530 mg (1.30 mmol) of tert-butyl [6-({2-[(tert-butoxycarbonyl)amino]ethyl}amino)-3-propanoyl-pyridin-2-yl]carbamate (Example 35A) were dissolved in 15 ml of a solution of hydrochloric acid in dioxane (4 M), and the mixture was stirred at RT for 20 h. After the reaction had gone to completion, the reaction mixture was diluted with diethyl ether (100 ml), and the precipitate was filtered off, washed with diethyl ether (100 ml) and dried. This gave 290 mg (91% of theory) of the product as a solid.

LCMS (method 6): Rₜ=1.15 min. (m/z=309 (M+H)⁺)

### Example 37A

### tert-Butyl 3-({6-[(tert-butoxycarbonyl)amino]-5-propanoylpyridin-2-yl}-amino)piperidine-1-carboxylate

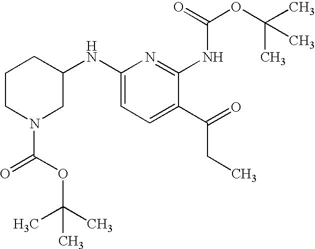

610 mg (3.0 mmol) of tert-butyl 3-aminopiperidine-1-carboxylate, 700 mg (2.3 mmol) of tert-butyl (6-chloro-3-propanoylpyridin-2-yl)carbamate (Example 34A) and 610 μl (3.5 mmol) of diisopropylethylamine were suspended in 7 ml of DMSO and heated at 90° C. in a microwave reactor for 45 min. The reaction mixture was diluted with ethyl acetate (100 ml) and washed with saturated aqueous ammonium chloride solution (3×40 ml) and then with saturated aqueous sodium bicarbonate solution (40 ml), and the organic phase was dried over magnesium sulphate and concentrated. The residue was chromatographed on silica gel (mobile phase: cyclohexane/ethyl acetate 5:1 to 1:1). 380 mg (35% of theory) of the product were isolated.

LCMS (method 6): Rₜ=2.42 min. (m/z=449 (M+H)⁺)

### Example 38A

### 1-[2-Amino-6-(piperidin-3-ylamino)pyridin-3-yl]propan-1-one hydrochloride

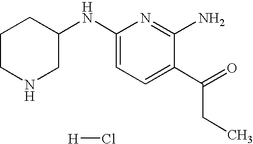

380 mg (0.85 mmol) of tert-butyl 3-({6-[(tert-butoxycarbonyl)amino]-5-propanoylpyridin-2-yl}amino)piperidine-1-carboxylate (Example 37A) were dissolved in 10 ml of a solution of hydrochloric acid in dioxane (4 M), and the mixture was stirred at RT for 20 h. After the reaction had gone to completion, the reaction mixture was diluted with diethyl ether (100 ml) and the precipitate was filtered off, washed with diethyl ether (100 ml) and dried. This gave 170 mg (70% of theory) of the product as a solid.

LCMS (method 9): Rₜ=0.95 min. (m/z=249 (M+H)⁺)

### Example 39A

### tert-Butyl 3-[(6-amino-5-nitropyridin-2-yl)amino]piperidine-1-carboxylate

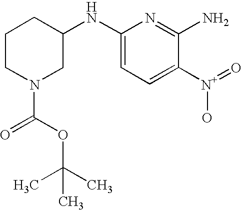

500 mg (2.11 mmol) of tert-butyl 3-aminopiperidine-1-carboxylate, 772 mg (4.22 mmol) of 2-amino-6-chloro-3-nitropyridine and 1.05 ml (6.34 mmol) of diisopropylethylamine were suspended in 18 ml of DMSO and heated in a microwave reactor at 120° C. for 45 min. The reaction mixture was purified by preparative reversed-phase HPLC (method 13). 600 mg (81% of theory) of the product were isolated as a solid.

LCMS (method 6): Rₜ=1.77 min. (m/z=338 (M+H)⁺)

### Example 40A

### 3-Nitro-N⁶-(piperidin-3-yl)pyridine-2,6-diamine hydrochloride

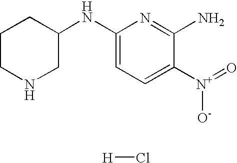

610 mg (1.62 mmol) of tert-butyl 3-[(6-amino-5-nitropyridin-2-yl)amino]piperidine-1-carboxylate (Example 39A) were dissolved in 40 ml of a solution of hydrochloric acid in dioxane (4 M), and the mixture was stirred at RT for 30 min. After the reaction had gone to completion, the solvent was removed completely. This gave 662 mg of the crude product.

LCMS (method 4): Rₜ=0.86 min. (m/z=238 (M+H)⁺)

### Example 41A

### Methyl 1-[2-(2,4-difluorophenyl)-2-oxoethyl]-1H-1,2,4-triazole-5-carboxylate

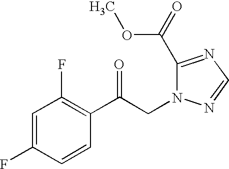

5.0 g (39.34 mmol) of methyl 1H-1,2,4-triazole-3-carboxylate were initially charged in 96 ml of acetone, and 7.87 g (41.3 mmol) of 2-chloro-1-(2,4-difluorophenyl)ethanone and 6.25 g (45.2 mmol) of potassium carbonate were added. The reaction mixture was stirred at RT for 20 h and then concentrated at 30° C. on a rotary evaporator. The residue was taken up in 600 ml of dichloromethane and 400 ml of water and washed twice with water and once with saturated sodium chloride solution. The organic phase was separated off, dried over magnesium sulphate and freed from the solvent. The crude product was chromatographed on a reversed phase, column type: Daisco C18, 10 μm Bio (DAN 300*100 nm). The mobile phase was a gradient of acetonitrile and water. 0.97 g (9% of theory) of the product was obtained as a solid.

LCMS (method 8): Rₜ=0.81 min. (m/z=282 (M+H)⁺)

¹H-NMR (400 MHz, DMSO-d₆): δ=8.26 (s, 1H), 8.04 (q, 1H), 7.57 (m, 1H), 7.33 (dt, 1H), 6.05 (s, 2H), 3.82 (s, 3H).

### Example 42A

### 6-(2,4-Difluorophenyl)[1,2,4]triazolo[1,5-a]pyrazin-8(7H)-one

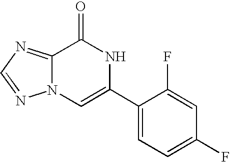

Under reflux, 965 mg (3.43 mmol) of methyl 1-[2-(2,4-difluorophenyl)-2-oxoethyl]-1H-1,2,4-triazole-5-carboxylate (Example 41A) and 2.65 g (34.3 mmol) of ammonium acetate were stirred in 25 ml of glacial acetic acid for 13 h. The reaction mixture was cooled and added to ice-water. The pH was adjusted to 4 using sodium bicarbonate, and the precipitated solid was filtered off with suction and dried. This gave 620 mg (73% of theory) of the product as a solid.

LCMS (method 9): Rₜ=1.35 min. (m/z=249 (M+H)⁺)

¹H-NMR (400 MHz, DMSO-d₆): δ=12.23 (s, 1H), 8.52 (s, 1H), 8.13 (s, 1H), 7.70 (q, 1H), 7.48 (dt, 1H), 7.26 (dt, 1H).

### Example 43A

### 8-Chloro-6-(2,4-difluorophenyl) [1,2,4]triazolo[1,5-a]pyrazine

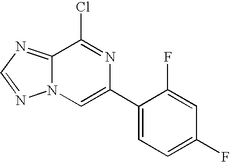

620 mg (2.5 mmol) of 6-(2,4-difluorophenyl)[1,2,4]triazolo[1,5-a]pyrazin-8(7H)-one (Example 42A) were initially charged in 20 ml of phosphoryl chloride, and 1.7 g (7.5 mmol) of benzyltriethylammonium chloride were added. The mixture was stirred at 120° C. for 3 h. Most of the phosphoryl chloride of the reaction mixture was removed under reduced pressure, and the mixture was then poured into ice-water. The precipitated solid was filtered off with suction, washed with water and dried. This gave 637 mg (96% of theory) of the product as a solid.

LCMS (method 6): Rₜ=1.81 min. (m/z=267 (M+H)⁺)

¹H-NMR (400 MHz, DMSO-d₆): δ=9.42 (s, 1H), 8.86 (s, 1H), 8.02 (m, 1H), 7.51 (m, 1H), 7.32 (dt, 1H).

### Example 44A

### tert-Butyl 3-[(4-amino-5-cyano-1,3-thiazol-2-yl)amino]piperidine-1-carboxylate

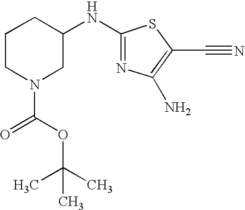

Analogously to the preparation of Example 26A, starting with 559 mg (2.36 mmol) of tert-butyl 3-aminopiperidine-1-carboxylate, after reaction with 500 mg (2.36 mmol) of 4-amino-2-(methylsulphonyl)-1,3-thiazole-5-carbonitrile, 212 mg (27% of theory) of the product were isolated as a solid.

LCMS (method 3): Rₜ=1.92 min. (m/z=324 (M+H)⁺)

¹H-NMR (400 MHz, DMSO-d₆): δ=8.35 (d, 1H), 6.68 (s, 2H), 3.56 (br s, 2H), 3.2-3.5 (m, 2H), 1.87 (m, 1H), 1.69 (m, 1H), 1.50 (m, 1H), 1.4 (m, 1H), 1.35 (s, 9H).

### Example 45A

### 4-Amino-2-(piperidin-3-ylamino)-1,3-thiazole-5-carbonitrile dihydrochloride

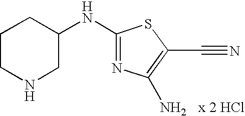

Analogously to the preparation of Example 30A, starting with 212 mg (0.65 mmol) of tert-butyl 3-[(4-amino-5-cyano-1,3-thiazol-2-yl)amino]piperidine-1-carboxylate, after reaction with 2 ml of hydrogen chloride in dioxane (4 M), 190 mg (99% of theory) of the product were isolated as a solid.

LCMS (method 9): Rₜ=0.71 min. (m/z=224 (M+H)⁺)

¹H-NMR (400 MHz, DMSO-d₆): δ=8.97-9.17 (m, 2H), 8.58 (d, 1H), 6.67 (br s, 1H), 3.88 (m, 1H), 3.32 (d, 1H), 3.10 (d, 1H), 2.77-2.93 (m, 2H), 1.97 (m, 1H), 1.85 (m, 1H), 1.67 (m, 1H), 1.49 (m, 1H).

### Example 46A

### tert-Butyl {3-[(6-amino-5-nitropyridin-2-yl)amino]propyl}carbamate

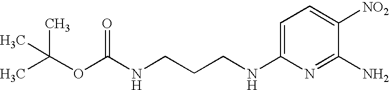

150.6 mg (0.864 mmol) of tert-butyl-(3-aminopropyl)carbamate, 300 mg (1.73 mmol) of 6-chloro-3-nitropyridine-2-amine and 173 mg (1.73 mmol) of potassium bicarbonate were suspended in 10 ml of DMF and heated at 90° C. for 16 h. Water was added, and the mixture was extracted three times with ethyl acetate. Purification of the crude product by preparative HPLC and drying under high vacuum gave 195 mg (65% of theory) of product.

LCMS (method 7): Rₜ=2.85 min. (m/z=312 (M+H)⁺).

¹H-NMR (400 MHz, DMSO-d₆): δ=8.12 (br s, 1H), 7.91 (d, 1H), 7.73 (br s, 1H), 6.84 (t, 1H), 5.93 (d, 1H), 4.09 (dd, 1H), 3.32 (m, 2H), 2.97 (q, 2H), 1.64 (m, 2H), 1.37 (s, 9H).

### Example 47A

### N⁶-(3-Aminopropyl)-3-nitropyridine-2,6-diamine dihydrochloride

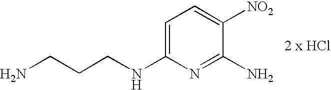

15.4 g (51.8 mmol) of tert-butyl {3-[(6-amino-5-nitropyridin-2-yl)amino]propyl}carbamate (Example 46A) were initially charged in 45 ml of dichloromethane and cooled to 0° C. 148 ml (400 mmol) of a solution of 4M hydrogen chloride in dioxane were then added, and the mixture was stirred at RT for 6 h. The precipitate was filtered off with suction, washed with diethyl ether and dried under high vacuum. This gave 12.49 g (89% of theory) of the product as a solid.

LCMS (method 9): Rₜ=0.53 min. (m/z=198 (M+H)⁺).

¹H-NMR (400 MHz, DMSO-d₆): δ=8.39 (br s, 1H), 8.13 (br s, 4H), 7.99 (d, 1H), 6.01 (d, 1H), 3.56 (m, 2H), 3.03 (m, 2H).

### Example 48A

### Methyl 1-[2-(2-chloro-4-fluorophenyl)-2-oxoethyl]-1H-1,2,4-triazole-5-carboxylate

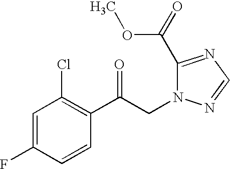

Analogously to the preparation of Example 18A, starting with 1.72 g (13.6 mmol) of methyl 1H-1,2,4-triazole-3-carboxylate, after reaction with 3.98 g (14.24 mmol) of 2-bromo-1-(2-chloro-4-fluorophenyl)ethanone, 1.1 g (21% of theory) of the product were isolated as a solid.

LCMS (method 8): Rₜ=1.01 min. (m/z=298 (M+H)⁺)

¹H-NMR (400 MHz, DMSO-d₆): δ=8.27 (s, 1H), 8.09 (dd, 1H), 7.67 (dd, 1H), 7.48 (dt, 1H), 6.13 (s, 2H), 3.85 (s, 3H).

### Example 49A

### 6-(2-Chloro-4-fluorophenyl) [1,2,4]triazolo[1,5-a]pyrazin-8(7H)-one

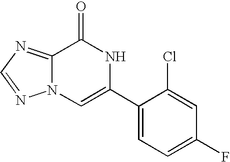

Analogously to the preparation of Example 19A, starting with 1.11 g (2.7 mmol) of methyl 1-[2-(2-chloro-4-fluorophenyl)-2-oxoethyl]-1H-1,2,4-triazole-5-carboxylate, after reaction with 2.07 g (26.8 mmol) of ammonium acetate, 655 mg (78% of theory) of the product were isolated as a solid.

LCMS (method 12): Rₜ=0.72 min. (m/z=265 (M+H)⁺)

¹H-NMR (400 MHz, DMSO-d₆): δ=12.18 (br s, 1H), 8.49 (s, 1H), 8.03 (s, 1H), 7.62-7.71 (m, 2H), 7.38 (dt, 1H).

### Example 50A

### 8-Chloro-6-(2-chloro-4-fluorophenyl) [1,2,4]triazolo[1,5-a]pyrazine

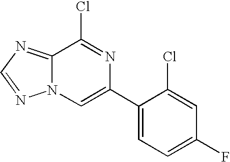

Analogously to the preparation of Example 20A, starting with 635 mg (2.4 mmol) of 6-(2-chloro-4-fluorophenyl)[1,2,4]triazolo[1,5-a]pyrazin-8(7H)-one, after reaction with 16.45 g (107.3 mmol) of phosphoryl chloride, 550 mg (82% of theory) of the product were isolated as a solid.

LCMS (method 8): Rₜ=1.16 min. (m/z=283 (M+H)⁺)

¹H-NMR (400 MHz, DMSO-d₆): δ=9.48 (s, 1H), 8.89 (s, 1H), 7.75 (dd, 1H), 7.68 (dd, 1H), 7.43 (dt, 1H).

### WORKING EXAMPLES

### Example 1

### 6-[(2-{[6-(2,4-Dichlorophenyl)[1,2,4]triazolo[3,4-f][1,2,4]triazin-8-yl]amino}ethyl)amino]pyridine-3-carbonitrile

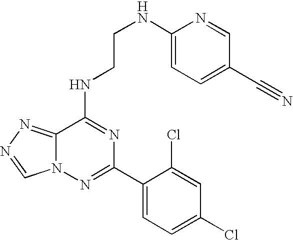

100 mg (0.33 mmol) of 8-chloro-6-(2,4-dichlorophenyl)[1,2,4]triazolo[3,4-f][1,2,4]triazine (Example 7A), 70 mg (0.43 mmol) of 6-[(2-aminoethyl)amino]pyridine-3-carbonitrile dihydrochloride (Example 2A) and 0.08 ml (0.5 mmol) of N,N-diisopropylethylamine were initially charged in 2 ml of DMSO. The mixture was stirred at 80° C. for 16 h. Ethyl acetate and 10% strength citric acid were added, and the reaction mixture was extracted. The organic phase was washed with sodium chloride solution and dried over magnesium sulphate. Purification by preparative HPLC (method 13) gave 16 mg (12% of theory) of the product as a solid.

LCMS (method 3): Rₜ=1.16 min. (m/z=426 (M+H)⁺)

¹H-NMR (400 MHz, DMSO-d₆): δ=9.76 (t, 1H), 9.56 (s, 1H), 8.34 (ss, 1H), 7.74 (br, s 1H), 7.62 (d, 1H), 7.57-7.50 (m, 2H), 6.52-6.41 (br, s, 1H), 3.76 (q, 2H), 3.62 (q, 2H).

### Example 2

### 2-Amino-6-[(2-{[6-(2,4-dichlorophenyl)[1,2,4]triazolo[3,4-f][1,2,4]triazin-8-yl]amino}ethyl)-amino]pyridine-3-carbonitrile

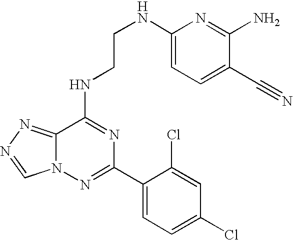

300 mg (0.99 mmol) of 8-chloro-6-(2,4-dichlorophenyl)[1,2,4]triazolo[3,4-f][1,2,4]triazine (Example 7A), 274.65 mg (1.09 mmol) of 2-amino-6-[(2-aminoethyl)amino]pyridine-3-carbonitrile dihydrochloride (Example 17A) and 1.4 ml (8 mmol) of N,N-diisopropylethylamine were initially charged in 5 ml of DMSO. The mixture was stirred at 80° C. for 16 h. Ethyl acetate and 10% strength citric acid were added, and the reaction mixture was extracted. The organic phase was washed with sodium chloride solution and dried over magnesium sulphate. The reaction mixture was purified by preparative HPLC (method 13). This gave 120 mg (27% of theory) of the product as a solid.

LCMS (method 3): Rₜ=2.24 min. (m/z=440 (M+H)⁺)

¹H-NMR (400 MHz, DMSO-d₆): δ=9.8 (t, 1H), 9.57 (s, 1H), 7.73 (ss, 1H), 7.66 (d, 1H), 7.53 (dd, 1H), 7.37 (br, s, 1H), 5.81 (br, s, 1H), 3.77 (s, 2H), 3.65 (s, 2H).

### Example 3

### 2-Amino-6-{[2-({6-[4-(trifluoromethyl)phenyl][1,2,4]triazolo[3,4-f][1,2,4]triazin-8-yl}amino)-ethyl]amino}pyridine-3-carbonitrile

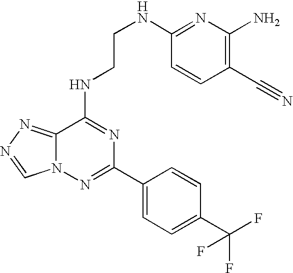

65 mg (0.22 mmol) of 8-chloro-6-[4-(trifluoromethyl)phenyl][1,2,4]triazolo[3,4-f][1,2,4]triazine (Example 10A), 60 mg (0.24 mmol) of 2-amino-6-[(2-aminoethyl)amino]pyridine-3-carbonitrile dihydrochloride (Example 17A) and 0.3 ml (1.75 mmol) of N,N-diisopropylethylamine were initially charged in 2 ml of DMSO. In a microwave reactor, the reaction mixture was irradiated at 140° C. for 30 min. The mixture was purified by preparative HPLC (method 13). This gave 17 mg (18% of theory) of the product as a solid.

LCMS (method 3): Rₜ=2.33 min. (m/z=441 (M+H)⁺)

¹H-NMR (400 MHz, DMSO-d₆): δ=9.77 (t, 1H), 9.57 (s, 1H), 8.27 (d, 2H), 7.82 (d, 2H), 7.63 (br, s, 1H), 7.24 (br, s, 1H), 5.73 (br, s, 1H), 3.92 (d, 2H), 3.63 (d, 2H).

### Example 4

### 6-{[2-({6-[4-(Trifluoromethyl)phenyl][1,2,4]triazolo[3,4-f][1,2,4]triazin-8-yl}amino)ethyl]-amino}pyridine-3-carbonitrile

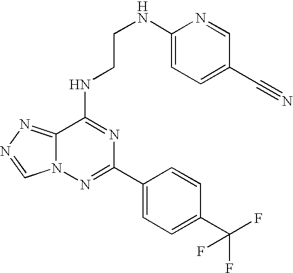

60 mg (0.2 mmol) of 8-chloro-6-[4-(trifluoromethyl)phenyl][1,2,4]triazolo[3,4-f][1,2,4]triazine (Example 10A), 39 mg (0.24 mmol) of 6-[(2-aminoethyl)amino]pyridine-3-carbonitrile dihydrochloride (Example 2A) and 0.1 ml (0.6 mmol) of N,N-diisopropylethylamine were initially charged in 2 ml of DMSO. In a microwave reactor, the reaction mixture was irradiated at 140° C. for 30 min. Purification by preparative HPLC (method 13) gave 40 mg (47% of theory) of the product as a solid.

LCMS (method 3): Rₜ=2.33 min. (m/z=426 (M+H)⁺)

¹H-NMR (400 MHz, DMSO-d₆): δ=9.71 (t, 1H), 9.56 (s, 1H), 8.47 (s, 1H), 8.21 (d, 2H), 7.81 (d, 3H), 7.50 (br, s, 1H), 6.41 (br, s, 1H), 3.92 (q, 2H), 3.68 (q, 2H).

### Example 5

### 6-[(2-{[6-(2,4-Dichlorophenyl)[1,2,4]triazolo[1,5-a]pyrazin-8-yl]amino}ethyl)amino]pyridine-3-carbonitrile

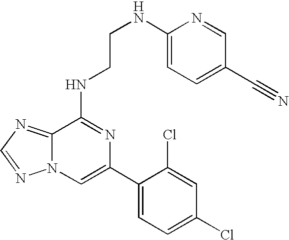

45 mg (0.15 mmol) of 8-chloro-6-(2,4-dichlorophenyl)[1,2,4]triazolo[1,5-a]pyrazine (Example 20A), 35 mg (0.18 mmol) of 6-[(2-aminoethyl)amino]pyridine-3-carbonitrile dihydrochloride (Example 2A) and 0.2 ml (1.2 mmol) of N,N-diisopropylethylamine were initially charged in 0.8 ml of DMSO. In a microwave reactor, the reaction mixture was irradiated at 140° C. for 30 min. Purification by preparative HPLC (method 13) gave 30 mg (47% of theory) of the product as a solid.

LCMS (method 6): Rₜ=2.05 min. (m/z=425 (M+H)⁺)

¹H-NMR (400 MHz, DMSO-d₆): δ=8.56 (s, 1H), 8.41 (s, 1H), 8.35 (ss, 1H), 8.22 (br, s 1H), 7.74 (ss, 1H), 7.71 (br, s, 1H), 6.62 (d, 1H), 7.56 (dd, 1H), 7.51 (dd, 1H), 6.50 (br, s, 1H), 3.72-3.56 (m, 4H).

### Example 6

### 1-{2-Amino-6-[(2-{[6-(2,4-dichlorophenyl)[1,2,4]triazolo[1,5-a]pyrazin-8-yl]amino}ethyl)amino]-pyridin-3-yl}-2,2,2-trifluoroethanone

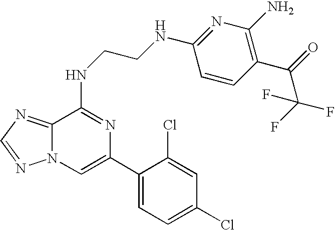

160 mg (0.53 mmol) of 8-chloro-6-(2,4-dichlorophenyl)[1,2,4]triazolo[1,5-a]pyrazine (Example 20A), 182 mg (0.64 mmol) of 1-{2-amino-6-[(2-aminoethyl)amino]pyridin-3-yl}-2,2,2-trifluoroethanone hydrochloride (Example 24A) and 0.74 ml (4.2 mmol) of N,N-diisopropylethylamine were initially charged in 3 ml of DMSO. In a microwave reactor, the reaction mixture was irradiated at 140° C. for 30 min. Purification by preparative HPLC (method 13) gave 186 mg (68% of theory) of the product as a solid.

LCMS (method 6): Rₜ=2.39 min. (m/z=511 (M+H)⁺)

¹H-NMR (400 MHz, DMSO-d₆): δ=8.58 (s, 1H), 8.54 (br s, 1H), 8.43 (s, 1H), 8.33 (t, 1H), 8.08 (t, 1H), 7.72 (d, 1H), 7.64 (d, 2H), 7.47 (t, 2H), 5.9 (d, 1H), 3.72 (m, 2H), 3.66 (m, 2H).

¹H-NMR (500 MHz, TFA-d): δ=8.82 (s, 1H), 8.35 (s, 2H), 7.68 (s, 1H), 7.55 (dd, 2H), 6.47 (br s, 1H), 4.43 (m, 2H), 4.20 (m, 2H).

### Example 7

### 1-(2-Amino-6-{[2-({6-[4-(trifluoromethyl)phenyl][1,2,4]triazolo[3,4-f][1,2,4]triazin-8-yl}amino)-ethyl]amino}pyridin-3-yl)-2,2,2-trifluoroethanone

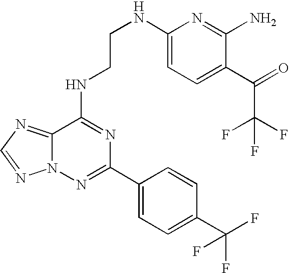

200 mg (0.67 mmol) of 8-chloro-6-[4-(trifluoromethyl)phenyl][1,2,4]triazolo[3,4-f][1,2,4]triazine (Example 10A), 210 mg (0.73 mmol) of 1-{2-amino-6-[(2-aminoethyl)amino]pyridin-3-yl}-2,2,2-trifluoroethanone hydrochloride (Example 24A) and 0.7 ml (4.0 mmol) of N,N-diisopropylethylamine were initially charged in 5 ml of DMSO. In a microwave reactor, the reaction mixture was irradiated at 130° C. for 45 min. Purification by preparative HPLC (method 13) gave 28 mg (8% of theory) of the product as a solid.

LCMS (method 3): Rₜ=2.56 min. (m/z=512 (M+H)⁺)

¹H-NMR (400 MHz, DMSO-d₆): δ=9.76 (t, 1H), 9.58 (s, 1H), 8.65 (br s, 1H), 8.19 (d, 2H), 7.80 (br s, 1H), 7.65 (d, 2H), 7.23 (m, 1H), 5.71 (d, 1H), 4.04 (m, 2H), 3.68 (m, 2H).

### Example 8

### 1-{2-Amino-6-[(2-{[6-(2,4-dichlorophenyl)[1,2,4]triazolo[3,4-f][1,2,4]triazin-8-yl]amino}ethyl)-amino]pyridin-3-yl}-2,2,2-trifluoroethanone

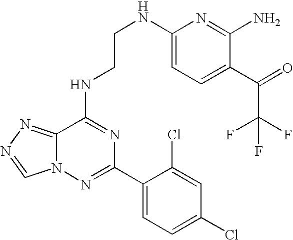

80 mg (0.27 mmol) of 8-chloro-6-(2,4-dichlorophenyl)[1,2,4]triazolo[3,4-f][1,2,4]triazine (Example 9A), 94 mg (0.29 mmol) of 1-{2-amino-6-[(2-aminoethyl)amino]pyridin-3-yl}-2,2,2-trifluoroethanone hydrochloride (Example 24A) and 280 μl (1.6 mmol) of N,N-diisopropyl-ethylamine were initially charged in 1 ml of DMSO. In a microwave reactor, the reaction mixture was irradiated at 120° C. for 30 min. The reaction mixture was purified by preparative HPLC (method 13). This gave 30 mg (22% of theory) of the product.

LCMS (method 3): Rₜ=2.52 min. (m/z=512 (M+H)⁺)

¹H-NMR (400 MHz, DMSO-d₆): δ=9.84 (m, 1H), 9.56 (s, 1H), 8.54 (br m, 1H), 8.11 (t, 1H), 7.70 (d, 1H), 7.67 (d, 1H), 7.64 (br m, 1H), 7.45 (dd, 2H), 5.88 (d, 1H), 3.82 (m, 2H), 3.67 (m, 2H).

¹H-NMR (500 MHz, pyridine-d₅): δ=11.27 (br s, 1H), 9.65 (s, 1H), 9.39 (s, 1H), 9.18 (t, 1H), 8.61 (br s, 1H), 7.91 (d, 1H), 7.68 (s, 1H), 7.67 (d, 1H), 7.45 (d, 1H), 6.01 (d, 1H), 4.12 (m, 2H), 4.05 (m, 2H).

### Example 9

### 1-[2-Amino-6-({1-[6-(2,4-dichlorophenyl) [1,2,4]triazolo[3,4-f][1,2,4]triazin-8-yl]piperidin-3-yl}amino)pyridin-3-yl]-2,2,2-trifluoroethanone

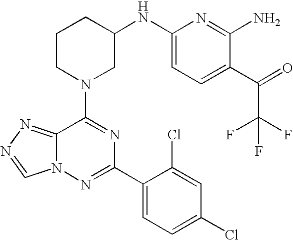

30 mg (0.09 mmol) of 8-chloro-6-(2,4-dichlorophenyl)[1,2,4]triazolo[3,4-f][1,2,4]triazine (Example 9A), 35 mg (0.11 mmol) of 1-[2-amino-6-(piperidin-3-ylamino)pyridin-3-yl]-2,2,2-trifluoroethanone hydrochloride (Example 33A) and 95 μl (0.5 mmol) of N,N-diisopropylethylamine were initially charged in 1 ml of DMSO. In a microwave reactor, the reaction mixture was stirred at 120° C. for 30 min. The reaction mixture was purified by preparative HPLC (method 13). This gave 41 mg (83% of theory) of the product.

LCMS (method 6): Rₜ=2.36 min. (m/z=552 (M+H)⁺)

The enantiomer separation of 1-[2-amino-6-({1-[6-(2,4-dichlorophenyl)[1,2,4]triazolo[3,4-f][1,2,4]triazin-8-yl]piperidin-3-yl}amino)pyridin-3-yl]-2,2,2-trifluoroethanone (Example 9) was carried out under the following conditions:

A sample of Example 9 (40 mg) was taken up in 2 ml of ethanol and chromatographed on a Daicel Chiralpak AS-H, 5 μm, 250 mm×20 mm column (flow rate: 15 ml/min; detection at 220 nm; injection volume: 700 μl; mobile phase: isohexane/(ethanol with 0.2% diethylamine) (50/50), temperature: 40° C.). Two fractions were isolated:

Example ENT-A-9: 10 mg of product in >99% ee were isolated.

Retention time 4.67 min

¹H-NMR (400 MHz, DMSO-d₆): δ=9.61 (s, 1H), 8.48 (s, br, 2H), 7.89 (d, 1H), 7.79 (dd, 1H), 7.5-7.61 (m, 1H), 7.44 (d, 1H), 7.34 (dd, 1H), 5.89 (d, 1H), 5.02 (d, 1H), 4.5-4.6 (m, 1H). 4.3-4.46 (m, 2H), 4.02 (d, 1H), 2.02-2.14 (m, 2H), 1.6-1.82 (m, 2H).

Example Ent-B-9: 14 mg of product in >99% ee were isolated.

Retention time 6.39 min

### Example 10

### 1-{2-Amino-6-[(2-{[6-(2,4-dichlorophenyl)[1,2,4]triazolo[3,4-f][1,2,4]triazin-8-yl]amino}ethyl)-amino]pyridin-3-yl}propan-1-one

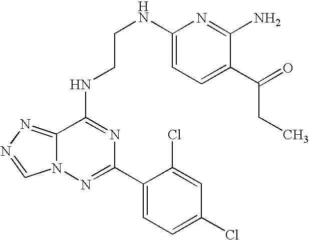

30 mg (0.09 mmol) of 8-chloro-6-(2,4-dichlorophenyl)[1,2,4]triazolo[3,4-f][1,2,4]triazine (Example 9A), 26 mg (0.11 mmol) of 1-{2-amino-6-[(2-aminoethyl)amino]pyridin-3-yl}propan-1-one hydrochloride (Example 36A) and 95 μl (0.5 mmol) of N,N-diisopropylethylamine were initially charged in 1 ml DMSO. In a microwave reactor, the reaction mixture was irradiated at 120° C. for 30 min. The reaction mixture was purified by preparative HPLC (method 13). This gave 13 mg (31% of theory) of the product.

LCMS (method 6): Rₜ=1.44 min. (m/z=472 (M+H)⁺)

¹H-NMR (400 MHz, DMSO-d₆): δ=9.84 (m, 1H), 9.59 (s, 1H), 7.82 (br m, 1H), 7.68 (m, 1H), 7.64 (d, 1H), 7.46 (dd, 1H), 5.75 (br m, 1H), 3.84 (m, 2H), 3.70 (m, 2H), 2.74 (m, 2H), 1.04 (t, 3H).

### Example 11

### 1-[2-Amino-6-({1-[6-(2,4-dichlorophenyl) [1,2,4]triazolo[3,4-f][1,2,4]triazin-8-yl]piperidin-3-yl}-amino)pyridin-3-yl]propan-1-one

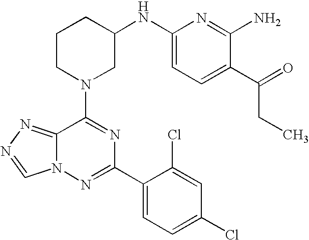

30 mg (0.09 mmol) of 8-chloro-6-(2,4-dichlorophenyl)[1,2,4]triazolo[3,4-f][1,2,4]triazine (Example 9A), 31 mg (0.11 mmol) of 1-[2-amino-6-(piperidin-3-ylamino)pyridin-3-yl]propan-1-one hydrochloride (Example 38A) and 95 μl (0.5 mmol) of N,N-diisopropylethylamine were initially charged in 1 ml DMSO. In a microwave reactor, the reaction mixture was irradiated at 120° C. for 30 min. The reaction mixture was purified by preparative HPLC (method 13). This gave 14 mg (30% of theory) of the product.

LCMS (method 6): Rₜ=1.72 min. (m/z=512 (M+H)⁺)

### Example 12

### N⁶-{1-[6-(2,4-Dichlorophenyl)[1,2,4]triazolo[3,4-f][1,2,4]triazin-8-yl]piperidin-3-yl}-3-nitro-pyridine-2,6-diamine hydrochloride

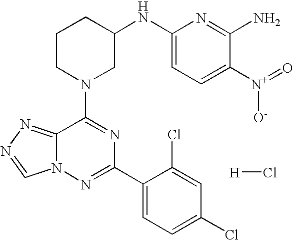

30 mg (0.09 mmol) of 8-chloro-6-(2,4-dichlorophenyl)[1,2,4]triazolo[3,4-f][1,2,4]triazine (Example 9A), 36 mg (0.12 mmol) of 3-nitro-N⁶-(piperidin-3-yl)pyridine-2,6-diamine hydrochloride (Example 40A) and 63 μl (0.36 mmol) of N,N-diisopropylethylamine were initially charged in 0.7 ml of DMSO. In a microwave reactor, the reaction mixture was irradiated at 120° C. for 30 min. The reaction mixture was purified by preparative HPLC (method 10). This gave 26 mg (54% of theory) of the product.

LC/MS (method 3): Rₜ=2.39 min, (m/z=501 (M+H)⁺)

The enantiomer separation of N⁶-{1-[6-(2,4-dichlorophenyl)[1,2,4]triazolo[3,4-f][1,2,4]triazin-8-yl]piperidin-3-yl}-3-nitropyridine-2,6-diamine hydrochloride (Example 12) was carried out under the following conditions:

A sample of Example 12 (18 mg) was taken up in 2.4 ml of ethanol and chromatographed on a Daicel Chiralpak AD-H, 5 μm, 250 mm×20 mm column (flow rate: 15 mL/min; detection at 220 nm; injection volume: 800 μl; mobile phase: isohexane/(ethanol with 0.2% diethylamine) (40/60), temperature: 40° C.). Two fractions were isolated:

Example Ent-A-12: 9 mg of product in >96% ee were isolated.

Retention time 7.27 min

¹H-NMR (400 MHz, TFA-d): δ=10.29 (s, 1H), 8.43 (d, 1H), 7.66 (d, 1H), 7.59 (s, 1H), 7.44 (d, 1H), 6.46 (d, 1H), 5.04-5.14 (m, 1H), 4.8-4.95 (m, 1H), 4.45-4.6 (m, 1H), 4.2-4.35 (m, 2H), 2.36-2.5 (m, 1H), 2.18-2.32 (m, 2H), 2.06-2.17 (m, 1H).

Example Ent-B-12: 7 mg of product in >99% ee were isolated.

Retention time 8.46 min

### Example 13

### 6-({1-[6-(2,4-Dichlorophenyl) [1,2,4]triazolo[3,4-f][1,2,4]triazin-8-yl]piperidin-3-yl}amino)pyridine-3-carbonitrile

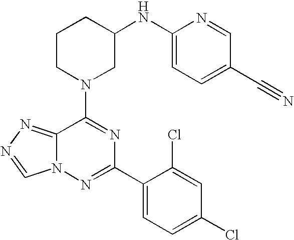

30 mg (0.09 mmol) of 8-chloro-6-(2,4-dichlorophenyl)[1,2,4]triazolo[3,4-f][1,2,4]triazine (Example 9A), 26 mg (0.11 mmol) of 6-(piperidin-3-ylamino)pyridine-3-carbonitrile hydrochloride (Example 30A) and 95 μl (0.5 mmol) of N,N-diisopropylethylamine were initially charged in 1 ml of DMSO. In a microwave reactor, the reaction mixture was irradiated at 120° C. for 30 min. The reaction mixture was purified by preparative HPLC (method 13). This gave 30 mg (72% of theory) of product.

LCMS (method 6): Rₜ=2.06 min. (m/z=466 (M+H)⁺)

¹H-NMR (400 MHz, TFA-d): δ=10.39 (s, 1H), 8.46 (s, 1H), 8.08 (s, 1H), 7.73 (m, 2H), 7.58 (s, 1H), 7.47 (s, 1H), 5.22 (m, 1H), 5.02 (m, 1H), 4.61 (m, 1H), 4.32-4.5 (m, 2H), 2.48-2.61 (m, 2H), 2.1-2.47 (m, 2H).

### Example 14

### 2-Amino-6-({1-[6-(2,4-dichlorophenyl)[1,2,4]triazolo[3,4-f][1,2,4]triazin-8-yl]piperidin-3-yl}-amino)pyridine-3-carbonitrile

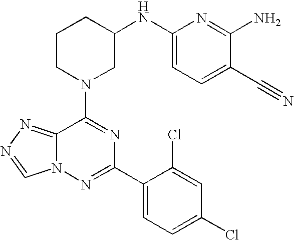

30 mg (0.09 mmol) of 8-chloro-6-(2,4-dichlorophenyl)[1,2,4]triazolo[3,4-f][1,2,4]triazine (Example 9A), 27 mg (0.11 mmol) of 2-amino-6-(piperidin-3-ylamino)pyridine-3-carbonitrile hydrochloride (Example 31A) and 95 μl (0.5 mmol) of N,N-diisopropylethylamine were initially charged in 1 ml of DMSO. In a microwave reactor, the reaction mixture was irradiated at 120° C. for 30 min. The reaction mixture was purified by preparative HPLC (method 13). This gave 30 mg (83% of theory) of the product.

LCMS (method 6): Rₜ=1.97 min. (m/z=481 (M+H)⁺)

### Example 15

### 4-Amino-2-[(2-{[6-(2,4-dichlorophenyl)[1,2,4]triazolo[1,5-a]pyrazin-8-yl]amino}ethyl)amino]-1,3-thiazole-5-carbonitrile

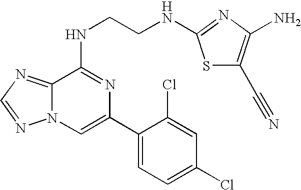

40 mg (0.13 mmol) of 8-chloro-6-(2,4-dichlorophenyl)[1,2,4]triazolo[1,5-a]pyrazine (Example 20A), 35 mg (0.16 mmol) of 4-amino-2-[(2-aminoethyl)amino]-1,3-thiazole-5-carbonitrile trifluoroacetate (Example 27A) and 0.14 ml (0.8 mmol) of N,N-diisopropylethylamine were initially charged in 1 ml of DMSO. In a microwave reactor, the reaction mixture was irradiated at 120° C. for 30 min. Purification by preparative HPLC (method 13) gave 29 mg (48% of theory) of the product as a solid.

LCMS (method 6): Rₜ=1.88 min. (m/z=446 (M+H)⁺)

¹H-NMR (400 MHz, DMSO-d₆): δ=8.58 (s, 1H), 8.47 (m, 1H), 8.44 (s, 1H), 8.23 (m, 1H), 7.74 (d, 1H), 7.65 (d, 1H), 7.53 (dd, 1H), 3.67 (m, 2H), 3.53 (m, 2H).

### Example 16

### 2-Amino-6-({1-[6-(2,4-dichlorophenyl)[1,2,4]triazolo[1,5-a]pyrazin-8-yl]piperidin-3-yl}amino)-pyridine-3-carbonitrile

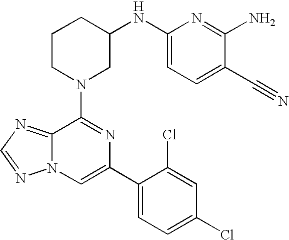

30 mg (0.10 mmol) of 8-chloro-6-(2,4-dichlorophenyl)[1,2,4]triazolo[1,5-a]pyrazine (Example 20A), 30 mg (0.12 mmol) of 2-amino-6-(piperidin-3-ylamino)pyridine-3-carbonitrile hydrochloride (Example 31A) and 78 mg (0.6 mmol) of N,N-diisopropylethylamine were initially charged in 1 ml of DMSO. In a microwave reactor, the reaction mixture was irradiated at 120° C. for 30 min. The reaction mixture was purified by preparative HPLC (method 13). This gave 43 mg (88% of theory) of the product as a solid.

LCMS (method 6): Rₜ=2.29 min. (m/z=480 (M+H)⁺)

¹H-NMR (400 MHz, DMSO-d₆): δ=8.58 (s, 1H), 8.55 (s, 1H), 7.74 (d, 1H), 7.67 (d, 1H), 7.54 (dd, 1H), 7.26 (d, 1H), 7.22 (br m, 1H), 6.32 (br m, 1H), 5.83 (br m, 1H), 4.34 (br m, 1H), 4.07 (br m, 1H), 2.01 (m, 1H), 1.90 (m, 1H), 1.62 (m, 2H). Some signals are obscured by the signal for water.

### Example 17

### 2-Amino-6-[(2-{[6-(2,4-dichlorophenyl)[1,2,4]triazolo[1,5-a]pyrazin-8-yl]amino}ethyl)amino]-pyridine-3-carbonitrile

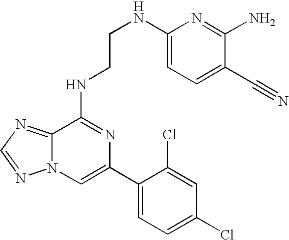

30 mg (0.10 mmol) of 8-chloro-6-(2,4-dichlorophenyl)[1,2,4]triazolo[1,5-a]pyrazine (Example 20A), 26 mg (0.12 mmol) of 2-amino-6-[(2-aminoethyl)amino]pyridine-3-carbonitrile dihydrochloride (Example 17A) and 78 mg (0.6 mmol) of N,N-diisopropylethylamine were initially charged in 1 ml of DMSO. In a microwave reactor, the reaction mixture was irradiated at 120° C. for 30 min. The reaction mixture was purified by preparative HPLC (method 13). This gave 30 mg (68% of theory) of the product as a solid.

LCMS (method 6): Rₜ=2.03 min. (m/z=440 (M+H)⁺)

¹H-NMR (400 MHz, DMSO-d₆): δ=8.57 (s, 1H), 8.44 (s, 1H), 8.46 (m, 1H), 7.76 (d, 1H), 7.65 (d, 1H), 7.53 (dd, 1H), 7.30 (d, 1H), 6.56 (br s, 1H), 5.83 (br m, 1H), 3.74 (br m, 4H).

### Example 18

### 6-({1-[6-(2,4-Dichlorophenyl) [1,2,4]triazolo[1,5-a]pyrazin-8-yl]piperidin-3-yl}amino)pyridine-3-carbonitrile hydrochloride

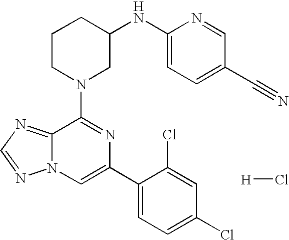

30 mg (0.10 mmol) of 8-chloro-6-(2,4-dichlorophenyl)[1,2,4]triazolo[1,5-a]pyrazine (Example 20A), 38 mg (0.13 mmol) of 6-(piperidin-3-ylamino)pyridine-3-carbonitrile hydrochloride (Example 30A) and 130 μl (0.75 mmol) of N,N-diisopropylethylamine were initially charged in 2 ml of DMSO. In a microwave reactor, the reaction mixture was irradiated at 120° C. for 30 min. The reaction mixture was purified by preparative HPLC (method 10). This gave 34 mg (54% of theory) of the product as a solid.

LC/MS (method 6): Rₜ=2.46 min, (m/z=465 (M+H)⁺)

¹H-NMR (400 MHz, DMSO-d₆): δ=8.56 (s, 1H), 8.54 (s, 1H), 8.34 (d, 1H), 7.74 (d, 1H), 7.61-7.7 (m, 2H), 7.58 (d, 1H), 7.52 (dd, 1H), 6.55 (d, 1H), 4.09 (br s, 1H), 4.65-4.95 (m, 2H), 2.04 (m, 2H), 1.93 (m, 2H), 1.59-1.72 (m, 4H).

### Example 19

### 1-[2-Amino-6-({1-[6-(2,4-dichlorophenyl) [1,2,4]triazolo[1,5-a]pyrazin-8-yl]piperidin-3-yl}amino)-pyridin-3-yl]-2,2,2-trifluoroethanone hydrochloride

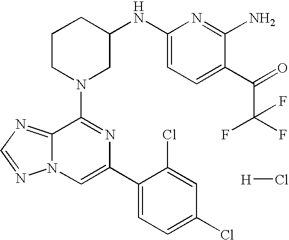

38 mg (0.13 mmol) of 8-chloro-6-(2,4-dichlorophenyl)[1,2,4]triazolo[1,5-a]pyrazine (Example 20A), 45 mg (0.14 mmol) of 1-[2-amino-6-(piperidin-3-ylamino)pyridin-3-yl]-2,2,2-trifluoro-ethanone hydrochloride (Example 33A) and 130 μl (0.75 mmol) of N,N-diisopropylethylamine were initially charged in 2 ml of DMSO. In a microwave reactor, the reaction mixture was irradiated at 120° C. for 30 min. The reaction mixture was purified by preparative HPLC (method 10). This gave 52 mg (71% of theory) of the product as a solid.

LC/MS (method 6): Rₜ=2.68 min, (m/z=551 (M+H)⁺)

¹H-NMR (400 MHz, DMSO-d₆): δ=8.57 (s, 1H), 8.56 (s, 1H), 8.50 (br s, 1H), 8.0 (d, 1H), 7.71 (d, 1H), 7.66 (d, 1H), 7.56 (br s, 1H), 7.41-7.51 (m, 2H), 5.93 (d, 1H), 4.68 (br s, 2H), 4.22-4.31 (m, 2H), 3.80 (dd, 1H), 2.05 (m, 1H), 1.95 (m, 1H), 1.65 (m, 2H).

The enantiomer separation of 1-[2-amino-6-({1-[6-(2,4-dichlorophenyl) [1,2,4]triazolo[1,5-a]pyrazin-8-yl]piperidin-3-yl}amino)pyridin-3-yl]-2,2,2-trifluoroethanone hydrochloride (Example 19) was carried out under the following conditions:

A sample of Example 19 (40 mg) was taken up in a warm mixture of 27 ml of ethanol and 3 ml of acetonitrile and chromatographed on a Daicel Chiralpak AD-H, 5 μm, 250 mm×20 mm column (flow rate: 15 mL/min; detection at 220 nm; injection volume: 500 μl; mobile phase: isohexane/2-propanol (75/25), temperature: 40° C.). Two fractions were isolated:

Example Ent-A-19: 14 mg of product in >99% ee were isolated.

Retention time 7.38 min

Example Ent-B-19: 17 mg of product in >98% ee were isolated.

Retention time 9.02 min

### Example 20

### 1-{2-Amino-6-[(2-{[6-(2,4-dichlorophenyl)[1,2,4]triazolo[1,5-a]pyrazin-8-yl]amino}ethyl)amino]-pyridin-3-yl}propan-1-one

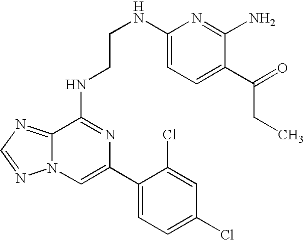

30 mg (0.10 mmol) of 8-chloro-6-(2,4-dichlorophenyl)[1,2,4]triazolo[1,5-a]pyrazine (Example 20A), 26 mg (0.12 mmol) of 1-{2-amino-6-[(2-aminoethyl)amino]pyridin-3-yl}propan-1-one hydrochloride (Example 36A) and 78 mg (0.6 mmol) of N,N-diisopropylethylamine were initially charged in 1 ml of DMSO. In a microwave reactor, the reaction mixture was irradiated at 120° C. for 30 min. The reaction mixture was purified by preparative HPLC (method 13). This gave 30 mg (68% of theory) of the product as a solid.

LCMS (method 6): Rₜ=1.66 min. (m/z=471 (M+H)⁺)

¹H-NMR (400 MHz, DMSO-d₆): δ=8.60 (s, 1H), 8.45 (s, 1H), 8.40 (br s, 1H), 7.78 (m, 1H), 7.71 (m, 1H), 7.62 (m, 1H), 7.48 (m, 1H), 6.25 (br, 1H), 5.80 (br m, 1H), 3.74 (m, 2H), 3.63 (m, 2H), 2.73 (m, 2H), 1.05 (t, 3H).

### Example 21

### 1-[2-Amino-6-({1-[6-(2,4-difluorophenyl) [1,2,4]triazolo[1,5-a]pyrazin-8-yl]piperidin-3-yl}amino)-pyridin-3-yl]-2,2,2-trifluoroethanone

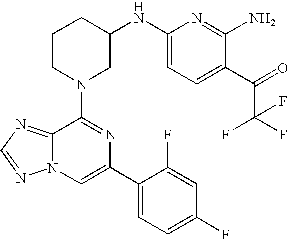

100 mg (0.38 mmol) of 8-chloro-6-(2,4-difluorophenyl)[1,2,4]triazolo[1,5-a]pyrazine (Example 43A), 146 mg (0.45 mmol) of 1-[2-amino-6-(piperidin-3-ylamino)pyridin-3-yl]-2,2,2-trifluoro-ethanone hydrochloride (Example 33A) and 523 μl (3 mmol) of N,N-diisopropylethylamine were initially charged in 4 ml of DMSO. In a microwave reactor, the reaction mixture was irradiated at 120° C. for 30 min. The reaction mixture was purified by preparative HPLC (method 13). This gave 175 mg (90% of theory) of the product as a solid.

LC/MS (method 8): Rₜ=1.49 min, (m/z=519 (M+H)⁺)

¹H-NMR (400 MHz, DMSO-d₆): δ=8.55 (s, 1H), 8.53 (s, 2H), 7.9-8.05 (m, 2H), 7.59 (s, 1H), 7.4-7.48 (d, 1H), 7.37 (t, 1H), 7.05 (t, 1H), 5.90 (d, 1H), 4.45-4.56 (m, 2H), 4.24-4.36 (m, 2H), 4.07-4.17 (m, 1H), 1.98-2.11 (m, 2H), 1.6-1.76 (m, 2H).

The enantiomer separation of 1-[2-amino-6-({1-[6-(2,4-difluorophenyl)[1,2,4]triazolo[1,5-a]pyrazin-8-yl]piperidin-3-yl}amino)pyridin-3-yl]-2,2,2-trifluoroethanone (Example 21) was carried out under the following conditions:

A sample of Example 21 (160 mg) was dissolved in 3 ml of ethanol and chromatographed on a Daicel Chiralpak AS-H, 5 μm, 250 mm×20 mm column (flow rate: 15 mL/min; detection at 220 nm; injection volume: 650 μl; mobile phase: isohexane/ethanol (70/30), temperature: 40° C.). Two fractions were isolated:

Example Ent-A-21: 53 mg of product in >99% ee were isolated.

Retention time 5.01 min

Example Ent-B-21: 82 mg of product in >99% ee were isolated.

Retention time 8.19 min

### Example 22

### 1-{2-Amino-6-[(2-{[6-(2,4-difluorophenyl) [1,2,4]triazolo[1,5-a]pyrazin-8-yl]amino}ethyl)amino]-pyridin-3-yl}-2,2,2-trifluoroethanone hydrochloride

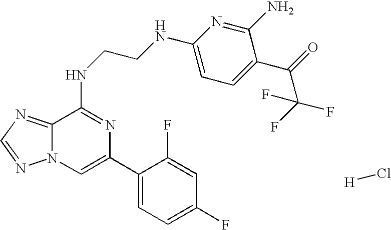

50 mg (0.19 mmol) of 8-chloro-6-(2,4-difluorophenyl)[1,2,4]triazolo[1,5-a]pyrazine (Example 43A), 64 mg (0.23 mmol) of 1-{2-amino-6-[(2-aminoethyl)amino]pyridin-3-yl}-2,2,2-trifluoroethanone hydrochloride (Example 24A) and 0.26 ml (1.5 mmol) of N,N-diisopropylethylamine were initially charged in 2 ml of DMSO. In a microwave reactor, the reaction mixture was irradiated at 140° C. for 30 min. Purification by preparative HPLC (method 10), gave 64 mg (63% of theory) of the product as a solid.

LCMS (method 6): Rₜ=2.29 min. (m/z=479 (M+H)⁺)

¹H-NMR (400 MHz, DMSO-d₆): δ=8.56 (br s, 2H), 8.44 (s, 1H), 8.33 (t, 1H), 8.11 (t, 1H), 8.0 (dd, 1H), 7.72 (m, 1H), 7.32-7.45 (m, 2H), 7.04 (dt, 1H), 5.85 (d, 1H), 3.79-3.87 (m, 2H), 3.62-3.72 (m, 2H).

### Example 23

### 4-Amino-2-({1-[6-(2,4-dichlorophenyl)[1,2,4]triazolo[1,5-a]pyrazin-8-yl]piperidin-3-yl}amino)-1,3-thiazole-5-carbonitrile

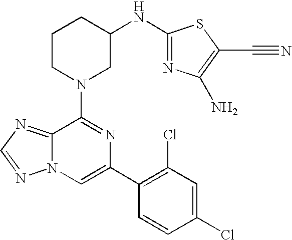

80 mg (0.27 mmol) of 8-chloro-6-(2,4-dichlorophenyl)[1,2,4]triazolo[1,5-a]pyrazine (Example 20A), 98.9 mg (0.29 mmol) of 4-amino-2-(piperidin-3-ylamino)-1,3-thiazole-5-carbonitrile dihydrochloride (Example 45A) and 279 μl (1.6 mmol) of N,N-diisopropylethylamine were initially charged in 2.2 ml of DMSO. In a microwave reactor, the reaction mixture was irradiated at 120° C. for 30 min. The reaction mixture was purified by preparative HPLC (method 13). This gave 69 mg (52% of theory) of the product as a solid.

LC/MS (method 3): Rₜ=2.57 min, (m/z=486 (M+H)⁺)

¹H-NMR (400 MHz, DMSO-d₆): δ=8.58 (s, 1H), 8.56 (s, 1H), 8.47 (d, 1H), 7.73 (d, 1H), 7.65 (d, 1H), 7.52 (dd, 1H), 6.67 (s, br, 1H), 4.4-4.6 (m, 2H), 4.05-4.2 (m, 1H), 3.9-4.04 (m, 1H), 3.77-3.90 (m, 1H), 1.98-2.07 (m, 1H), 1.84-1.95 (m, 1H), 1.55-1.74 (m, 2H).

### Example 24

### N⁶-(2-{[6-(2,4-Dichlorophenyl) [1,2,4]triazolo[1,5-a]pyrazin-8-yl]amino}ethyl)-3-nitropyridine-2,6-diamine trifluoroacetate

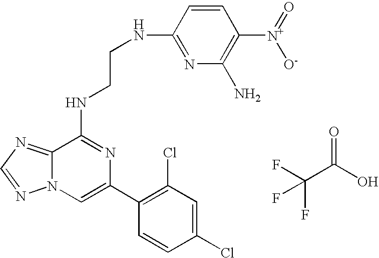

50 mg (0.17 mmol) of 8-chloro-6-(2,4-dichlorophenyl)[1,2,4]triazolo[1,5-a]pyrazine (Example 20A), 58.2 mg (0.2 mmol) of N⁶-(2-aminoethyl)-3-nitropyridine-2,6-diamine dihydrochloride (Example 47A) and 233 μl (1.34 mmol) of N,N-diisopropylethylamine were initially charged in 3.1 ml of DMSO. In a microwave reactor, the reaction mixture was irradiated at 140° C. for 30 min. The reaction mixture was purified by preparative HPLC (method 11). This gave 40.7 mg (42% of theory) of the product as a solid.

LC/MS (method 3): Rₜ=2.51 min, (m/z=460 (M+H)⁺)

¹H-NMR (400 MHz, DMSO-d₆): δ=8.58 (s, 1H), 8.43 (s, 1H), 8.31 (t, 1H), 8.15 (br s, 1H), 8.07 (t, 1H), 7.88 (d, 1H), 7.72 (s, 1H), 7.68-7.80 (br s, 1H), 7.63 (d, 1H), 7.46 (d, 1H), 5.88 (d, 1H), 3.72 (m, 2H), 3.64 (m, 2H).

### Example 25

### 1-{2-Amino-6-[(2-{[6-(2-chloro-4-fluorophenyl)[1,2,4]triazolo[1,5-a]pyrazin-8-yl]amino}ethyl)-amino]pyridin-3-yl}-2,2,2-trifluoroethanone hydrochloride

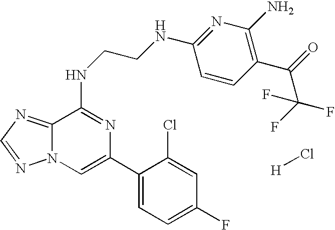

80 mg (0.28 mmol) of 8-chloro-6-(2-chloro-4-fluorophenyl)[1,2,4]triazolo[1,5-a]pyrazine (Example 50A), 97 mg (0.34 mmol) of 1-{2-amino-6-[(2-aminoethyl)amino]pyridin-3-yl}-2,2,2-trifluoroethanone hydrochloride (Example 24A) and 394 μl (2.26 mmol) of N,N-diisopropyl-ethylamine were initially charged in 2 ml of DMSO. In a microwave reactor, the reaction mixture was irradiated at 120° C. for 30 min. The reaction mixture was purified by preparative HPLC (method 10). This gave 47 mg (31% of theory) of the product as a solid.

LC/MS (method 8): Rₜ=1.40 min, (m/z=495 (M+H)⁺)

¹H-NMR (400 MHz, DMSO-d₆): δ=8.58 (s, 1H), 8.40 (s, 1H), 8.32 (t, 1H), 8.22 (br s, 1H), 7.72 (m, 1H), 7.68 (dd, 1H), 7.54 (dd, 1H), 7.48 (m, 1H), 7.26 (dt, 1H), 5.91 (d, 1H), 3.73 (m, 2H), 3.67 (m, 2H).

### Example 26

### N⁶-(2-{[6-(2,4-Difluorophenyl) [1,2,4]triazolo[1,5-a]pyrazin-8-yl]amino}ethyl)-3-nitropyridine-2,6-diamine

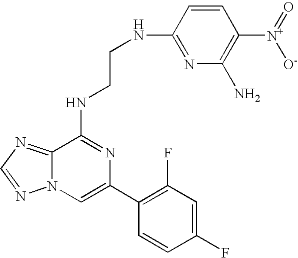

50 mg (0.19 mmol) of 8-chloro-6-(2,4-difluorophenyl)[1,2,4]triazolo[1,5-a]pyrazine (Example 43A), 65.4 mg (0.23 mmol) of N⁶-(2-aminoethyl)-3-nitropyridine-2,6-diamine dihydrochloride (Example 47A) and 261 μl (1.5 mmol) of N,N-diisopropylethylamine were initially charged in 3.6 ml of DMSO. In a microwave reactor, the reaction mixture was irradiated at 140° C. for 30 min. The reaction mixture was purified by preparative HPLC (method 13). This gave 2.3 mg (3% of theory) of the product as a solid.

LC/MS (method 3): Rₜ=2.34 min, (m/z=428 (M+H)⁺)

¹H-NMR (400 MHz, DMSO-d₆): δ=8.56 (s, 1H), 8.44 (s, 1H), 8.28 (m, 2H), 7.98 (m, 3H), 7.84 (d, 1H), 7.36 (dt, 1H), 7.08 (t, 1H), 5.86 (d, 1H), 3.83 (m, 2H), 3.68 (m, 2H).

### Example 27

### 2-Amino-6-[(2-{[6-(2,4-difluorophenyl)[1,2,4]triazolo[1,5-a]pyrazin-8-yl]amino}ethyl)amino]-pyridine-3-carbonitrile hydrochloride

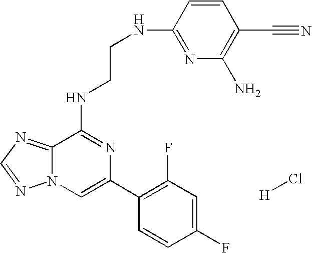

50 mg (0.19 mmol) of 8-chloro-6-(2,4-difluorophenyl)[1,2,4]triazolo[1,5-a]pyrazine (Example 43A), 66.2 mg (0.23 mmol) of 2-amino-6-[(2-aminoethyl)amino]pyridine-3-carbonitrile dihydrochloride (Example 17A) and 261 μl (1.5 mmol) of N,N-diisopropylethylamine were initially charged in 3.6 ml of DMSO. In a microwave reactor, the reaction mixture was irradiated at 140° C. for 30 min. The reaction mixture was purified by preparative HPLC (method 10). This gave 23 mg (26% of theory) of the product as a solid.

LC/MS (method 6): Rₜ=1.88 min, (m/z=408 (M+H)⁺)

¹H-NMR (400 MHz, DMSO-d₆): δ=8.56 (s, 1H), 8.44 (s, 1H), 8.29 (m, 1H), 7.98 (dd, 1H), 7.38 (m, 2H), 7.18 (dt, 1H), 5.83 (br s, 1H), 4.08 (br s, 3H), 3.79 (m, 2H), 3.65 (m, 2H).

### Example 28

### 6-[(2-{[6-(2,4-Difluorophenyl)[1,2,4]triazolo[1,5-a]pyrazin-8-yl]amino}ethyl)amino]pyridine-3-carbonitrile hydrochloride

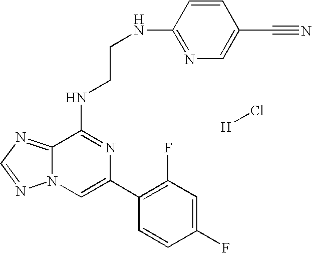

50 mg (0.19 mmol) of 8-chloro-6-(2,4-difluorophenyl)[1,2,4]triazolo[1,5-a]pyrazine (Example 43A), 62.2 mg (0.23 mmol) of 6-[(2-aminoethyl)amino]pyridine-3-carbonitrile dihydrochloride (Example 2A) and 261 μl (1.5 mmol) of N,N-diisopropylethylamine were initially charged in 3.6 ml of DMSO. In a microwave reactor, the reaction mixture was irradiated at 140° C. for 30 min. The reaction mixture was purified by preparative HPLC (method 10). This gave 59 mg (72% of theory) of the product as a solid.

LC/MS (method 8): Rₜ=1.19 min, (m/z=393 (M+H)⁺)

¹H-NMR (400 MHz, DMSO-d₆): δ=8.56 (s, 1H), 8.44 (s, 2H), 8.24 (m, 1H), 8.06 (br s, 1H), 7.98 (dd, 1H), 7.62 (br s, 1H), 7.39 (t, 1H), 7.19 (t, 1H), 6.55 (br s, 1H), 3.78 (m, 2H), 3.66 (m, 2H).

### B) ASSESSMENT OF THE PHYSIOLOGICAL ACTIVITY

The suitability of the compounds according to the invention for treating haematological disorders can be shown in the following assay systems:

### In Vitro Assay

The inhibitory activity of active substances is determined in a biochemical assay. The ingredients required for this purpose are mixed in a black 384-well microtitre plate with transparent base (from Greiner, catalogue number 781092). The requirements in this connection for each well of the 384-well microtitre plate are 5 nM GSK3β (from Upstate, catalogue number 14-306), 40 μM GSK3β substrate GSM (sequence H-RRRPASVPPSPSLSRHS-(pS)-HQRR, from Upstate, catalogue number 2-533), 30 μM nicotinamide adenine dinucleotide NADH (Roche Diagnostics, catalogue number 10107735), 50 μM adenosine triphosphate ATP (from Sigma, catalogue number A7966), 2 mM phosphoenolpyruvate (from Roche, catalogue number 128112), and also about 1 U/ml of pyruvate kinase and about 1 U/ml of lactate dehydrogenase, which are present together in a stock formulation (from Roche, catalogue number 10737291001, suspension with about 450 U/ml of pyruvate kinase activity, about 450 U/ml of lactate dehydrogenase activity in 3.2 mM ammonium sulphate solution pH 6), where 1 unit pyruvate kinase converts, at pH 7.6 and 37° C., 1 μmol of phosphoenolpyruvate into pyruvate per minute, and where 1 unit of lactate dehydrogenase reduces, at pH 7.5 and 37° C., 1 μmol of pyruvate to lactate per minute. The required reaction buffer in which the biochemical reaction takes place consists of 50 mM Trizma hydrochloride Tris-HCl pH: 7.5 (from Sigma, catalogue number T3253), 5 mM magnesium chloride MgCl₂ (from Sigma, catalogue number M8266), 0.2 mM DL-dithiothreitol DTT (from Sigma, catalogue number D9779), 2 mM ethylenediaminetetraacetic acid EDTA (from Sigma, catalogue number E6758), 0.01% Triton X-100 (from Sigma, catalogue number T8787) and 0.05% bovine serum albumin BSA (from Sigma, catalogue number B4287).

Active substances are dissolved in dimethyl sulphoxide DMSO (from Sigma, catalogue number D8418) in a concentration of 10 mM. Active substances are added in serial concentrations of 10 μM, 1 μM, 0.1 μM, 0.01 μM, 0.001 μM, 0.0001 μM, 0.00001 μM, 0.000001 μM to the mixtures of the biochemical reaction. As control, dimethyl sulphoxide is added instead of substance in a final concentration of 0.1%.

The reaction is incubated at 30° C. for 2 hours and then the resulting fluorescence is measured in a Tecan Safire-XFLUOR4 instrument, version V4.50 (serial number 12901300283) with the specifications: measurement mode—fluorescence measured from below, excitation wavelength 340 nm, emission wavelength 465 nm, slit width excitation 5 nm, slit width emission 5 nm, gain mode 120, delay 0 μs, number of light flashes per measurement 3, and an integration time of 40 μs.

The GSK3β activity is measured in fluorescence units, with the values of uninhibited kinase being set equal to 100% and those of completely inhibited kinase being set equal to 0%. The activity of the active substances is calculated in relation to these 0% and 100%.

Table A shows IC₅₀ values which were determined using the assay described above.

TABLE A

| Example No. | IC₅₀ [nM] |
|---|---|
| 6 | 13 |
| 8 | 9.1 |
| Ent-B-9 | 1.3 |
| 11 | 4.4 |
| Ent-B-12 | 4.3 |
| 15 | 3.2 |

### CD34+ Proliferation Assays for Testing GSK3β Inhibitors

Adult haematopoietic stem cells are characterized by the specific expression of membrane-associated proteins. These surface markers are provided with an appropriate number appropriate for their molecular weight. This class also includes the molecule which is referred to as CD34 and which serves for the identification, characterization and isolation of adult haematopoietic stem cells. These stem cells can moreover be isolated from bone marrow, peripheral blood or umbilical cord blood. These cells have limited viability in in vitro cultures but can be stimulated to proliferation and differentiation by various additions to the culture medium. CD34-positive cells are used here in order to test the influence of substances on the activity of glycogen synthase kinase 3. For this purpose, in a first step, mononuclear cells are isolated from umbilical cord blood by differential centrifugation steps.

For this purpose, umbilical cord blood is diluted 1:4 with phosphate-buffered saline solution. 50 millilitre centrifugation vessels are charged with 17 millilitres of Ficoll (density 1.077, Ficoll Paque Plus; Pharmacia, catalogue number 17-1440-02). 30 millilitres of the 1:4 diluted umbilical cord blood are layered thereon and then centrifuged at 400×g at room temperature for 30 minutes. The brakes of the centrifuge are disengaged during this. Owing to the centrifugation, the mononuclear cells collect in the interphase. This is removed with the aid of a 30 millilitre pipette and transferred into a new 50 millilitre centrifugation vessel, and the volume is then made up to 30 ml with phosphate-buffered saline solution. These cells are centrifuged at 300×g with the brake engaged at room temperature for 10 minutes. The supernatant is discarded and the resulting cell pellet is resuspended in 30 millilitres of phosphate-buffered saline solution. These cells are again centrifuged at 200×g with brake engaged at 20° C. for 15 minutes.

To isolate the CD34-positive cells, the enriched mononuclear cells are resuspended in a concentration of 1×10⁸ cells per 300 microlitres of MACS buffer (0.5% endotoxin-free bovine serum albumin in phosphate-buffered saline solution). 100 microlitres of FCR blocking reagent (Miltenyi Biotec, catalogue number 130-046-702) and 100 microlitres of CD34 microbeads (Miltenyi Biotec, catalogue number 130-046-702) are added. This suspension is incubated at 4° C. for 30 minutes. The cells are then diluted with 20 times the volume of MACS buffer and centrifuged at 300×g for 10 minutes. The supernatant is discarded and the cells are resuspended in 500 microlitres of MACS buffer. The cells treated in this way are loaded onto an LS column (Miltenyi Biotec, catalogue number 130-042-401) and purified using a Midi MACS magnet (Miltenyi Biotec, catalogue number 130-042-303).

The number of CD34-positive cells is determined by counting the cells using a Neubauer chamber. The purity of the cells is determined by standard protocols using the fluorescent activated cell sorting method (Becton Dickinson, BD FACS™ Sample Prep Assistant SPAII Upgrade Kit, catalogue number 337642).

To determine the influence of modulating the GSK3 activity, CD34-positive cells are incubated in a 96-well microtitre plate at 37° C. and 5% carbon dioxide for 7 days and then the proliferation rates are determined on the basis of the cell counts.

For this purpose, 5000 CD34-positive cells are taken up in 100 microlitres of IMDM medium (Life Technology, catalogue number 12440-046), 10% foetal calf serum (Life Technology, catalogue number 10082-139) and 20 nanograms per millilitre of stem cell factor (R&D, catalogue number 255-SC-010) in each well of a 96 U-bottom well microtitre plate (Greiner Bio-One, catalogue number 650 180). In addition, the cells are also mixed with various concentrations of substances dissolved in dimethyl sulphoxide (Sigma Aldrich, catalogue number D5879-1L). This entails 4 wells in each case with the stated cell count of 5000 CD34-positive cells per well being provided with 10 micromol, 4 wells with 5 micromol, 4 wells with 2.5 micromol, 4 wells with 1.25 micromol, 4 wells with 0.625 micromol, 4 wells with 0.3125 micromol, 4 wells with 0.156 micromol, 4 wells with 0.078 micromol and as control 4 wells with 0.1% dimethyl sulphoxide as final concentration.

These cells treated in this way are incubated in a cell culture incubator at 37° C. and 5% carbon dioxide for 7 days. The proliferation rate is determined by renewed counting of the cells using a Neubauer counting chamber, with the cells provided only with the stem cell factor being set as 100% value, and all other values being related to this value.

### In Vivo Assay

The investigations of the in vivo effect of the compounds according to the invention take place using 6-week old male C57BL/6 mice (Charles River, Sulzfeld, Germany) weighing 18-22 g. These animals are kept appropriate for the species with 12-hour light and dark cycles under constant climatic conditions and with water and mouse feed ad libitum. The concentrations of chemotherapeutics used are administered to the animals in accordance with the manufacturers' statements by intraperitoneal (i.p.) injections in the caudal third of the abdomen. The same procedure is applied to the substances relevant to the invention. Blood samples are taken from the retrobulbar venous plexus using Pasteur pipettes. The number of neutrophilic granulocytes is determined completely automatically using flow cytometry systems.

### CYP Inhibition Test

The ability of substances to inhibit CYP1A2, CYP2C8, CYP2C9, CYP2D6 and CYP3A4 in humans is examined using pooled human liver microsomes as enzyme source in the presence of standard substrate (vide infra) which form CYP isoform-specific metabolites. The inhibitory effects are studied at six different concentrations of the test compounds (1.5, 3.1, 6.3, 12.5, 25 and 50 μM) and compared to the extent of the CYP isoform-specific metabolite formation of the standard substrates in the absence of test compounds, and the corresponding IC₅₀ values are calculated. A standard inhibitor which specifically inhibits a single CYP isoform serves as control of the results obtained.

### Procedure:

The incubation of phenacetin, amodiaquine, diclofenac, dextromethorphan or midazolam with human liver microsomes in the presence of in each case six different concentrations of a test compound (as potential inhibitor) is carried out on a workstation (Tecan, Genesis, Crailsheim, Germany). Standard incubation mixtures comprise 1.3 mM NADP, 3.3 mM MgCl₂×6H₂O, 3.3 mM glucose 6-phosphate, glucose 6-phosphate dehydrogenase (0.4 U/ml) and 100 mM phosphate buffer (pH 7.4) in a total volume of 200 μl. Test compounds are preferably dissolved in acetonitrile. 96-Well plates are incubated for a defined period of time at 37° C. with pooled human liver microsomes. The reactions are stopped by addition of 100 μl of acetonitrile comprising a suitable internal standard. Precipitated proteins are removed by centrifugation, and the supernatants are combined and analysed by LC-MS/MS.

Determination of the Solubility

### Reagents Required:

PBS buffer pH 6.5: 61.86 g of sodium chloride p.a. (for example from Merck, Art. No. 1.06404.1000), 39.54 g of sodium dihydrogen phosphate p.a. (for example from Merck, Art. No. 1.06346.1000) and 83.35 g of 1 N aqueous sodium hydroxide solution (for example from Bernd Kraft GmbH, Art. No. 01030.4000) are weighed out into a 1 litre measuring flask and made up with water, and the mixture is stirred for about 1 hour. 500 ml of this solution are transferred into a 5 litre measuring flask and made up with water. The pH is adjusted to 6.5 using 1 N aqueous sodium hydroxide solution.
Dimethyl sulphoxide (for example from Baker, Art. No. 7157.2500)
Distilled water
Acetonitrile Chromasolv (for example Riedel-de Haen Art. No. 34851)
50% strength formic acid p.a. (for example Fluka Art. No. 09676)

### Preparation of the Starting Solution:

At least 1.5 mg of the test substance are weighed out accurately into a Wide Mouth 10 mm Screw V-Vial (from Glastechnik Gräfenroda GmbH, Art. No. 8004-WM-H/V 15μ) with fitting screw cap and septum, DMSO is added to give a concentration of 50 mg/ml and the mixture is vortexed for 30 minutes.

### Preparation of the Calibration Solutions:

The required pipetting steps are carried out in a 1.2 ml Deep Well Plate (DWP) with 96 wells (e.g. HJ-Bioanalytik GmbH Art. No. 850289) using a liquid handling robot. The solvent used is a mixture of acetonitrile Chromasolv/water 8:2.

Preparation of the starting solutionfor calibration solutions (stock solution): 833 μl of the solvent mixture are added to 10 μl of the initial solution (concentration=600 μg/ml), and the mixture is homogenized. For each test substance, 1:100 dilutions are prepared in separate DWPs, and the dilutions for their part are homogenized. One of the 1:100 dilutions is used for preparing the calibration solutions, the second dilution is used for optimizing the MS/MS parameter.

Calibration solution 5 (600 ng/ml): 270 μl of solvent mixture are added to 30 μl of the stock solution, and the mixture is homogenized.

Calibration solution 4 (60 ng/ml): 270 μl of solvent mixture are added to 30 μl of calibration solution 5, and the mixture is homogenized.

Calibration solution 3 (12 ng/ml): 400 μl of solvent mixture are added to 100 μl of calibration solution 4, and the mixture is homogenized.

Calibration solution 2 (1.2 ng/ml): 270 μl of solvent mixture are added to 30 μl of calibration solution 3, and the mixture is homogenized.

Calibration solution 1 (0.6 ng/ml): 150 μl of solvent mixture are added to 150 μl of calibration solution 2, and the mixture is homogenized.

### Preparation of the Sample Solutions:

The required pipetting steps are carried out in a 1.2 ml DWP with 96 wells (e.g. HJ-Bioanalytik GmbH Art. No. 850289) using a liquid handling robot.

1000 μl of PBS buffer pH 6.5 are added to 10.1 μl of the stock solution.

### Procedure:

The required pipetting steps are carried out in a 1.2 ml DWP with 96 wells (e.g. HJ-Bioanalytik GmbH Art. No. 850289) using a liquid handling robot.

Using a temperature-adjustable shaker (e.g. from Eppendorf Thermomixer comfort Art. No. 5355000.011), the sample solutions prepared in this manner are shaken at 20° C. and 1400 rpm for 24 hours. From these solutions, in each case 180 μl are removed and transferred into Beckman polyallomer centrifuge tubes (Art. No. 343621). These solutions are centrifuged at about 223 000×g for 1 hour (e.g. from Beckman Optima L-90K Ultracentrifuge with type 42.2 Ti rotor at 42 000 rpm). From each sample solution, 100 μl of the supernatant are removed and diluted 1:10 and 1:1000 with PBS buffer 6.5.

### Analysis:

The samples are analysed by HPLC/MS-MS. Quantification is carried out using a five point calibration curve of the test compound. The solubility is expressed in mg/l. Analysis sequence: 1) blank (solvent mixture); 2) calibration solution 0.6 ng/ml; 3) calibration solution 1.2 ng/ml; 4) calibration solution 12 ng/ml; 5) calibration solution 60 ng/ml; 6) calibration solution 600 ng/ml; 7) blank (solvent mixture); 8) sample solution 1:1000; 7) sample solution 1:10.

### HPLC/MS-MS Method

HPLC: Agilent 1100, quat. pump (G1311A), autosampler CTC HTS PAL, degasser (G1322A) and column thermostat (G1316A); column: Oasis HLB 20 mm×2.1 mm, 25 μl; temperature: 40° C.; mobile phase A: water+0.5 ml of formic acid/l; mobile phase B: acetonitrile+0.5 ml of formic acid/l; flow rate: 2.5 mL/min; stop time 1.5 min; gradient: 0 min 95% A, 5% B; ramp: 0-0.5 min 5% A, 95% B; 0.5-0.84 min 5% A, 95% B; ramp: 0.84-0.85 min 95% A, 5% B; 0.85-1.5 min 95% A, 5% B.

MS/MS: WATERS Quattro Micro Tandem MS/MS; Z-Spray API interface; HPLC-MS initial splitter 1:20; measurement in the ESI mode.

For each test substance, the instrument parameters are automatically optimized by injection of the stock solution described further above (second 1:100 dilution) using the MassLynx/QuanOptimize software.

### C) EXEMPLARY EMBODIMENTS OF PHARMACEUTICAL COMPOSITIONS

The substances according to the invention can be converted into pharmaceutical preparations in the following ways:

### Tablet:

### Composition:

100 mg of the compounds of Example 1, 50 mg of lactose (monohydrate), 50 mg of maize starch, 10 mg of polyvinylpyrrolidone (PVP 25) (from BASF, Germany) and 2 mg of magnesium stearate.

Tablet weight 212 mg. Diameter 8 mm, radius of curvature 12 mm.

### Production:

The mixture of the compound of Example 1, lactose and starch is granulated with a 5% strength solution (m/m) of the PVP in water. The granules are dried and then mixed with magnesium stearate for 5 min. This mixture is compressed with a conventional tablet press (see above for format of the tablet).

### Oral Suspension:

### Composition:

1000 mg of the compound of Example 1, 1000 mg of ethanol (96%), 400 mg of Rhodigel (xanthan gum) (from FMC, USA) and 99 g of water.

10 ml of oral suspension correspond to a single dose of 100 mg of the compound according to the invention.

### Production:

The Rhodigel is suspended in ethanol, and the compound of Example 1 is added to the suspension. The water is added while stirring. The mixture is stirred for about 6 h until the swelling of the Rhodigel is complete.

Solution which can be Administered Intravenously:

### Composition:

1 mg of the compound of Example 1, 15 g of polyethylene glycol 400 and 250 g of water for injections.

### Production:

The compound of Example 1 is dissolved together with polyethylene glycol 400 in the water by stirring. This solution is sterilized by filtration (pore diameter 0.22 μm) and dispensed under aseptic conditions into heat-sterilized infusion bottles. These are closed with infusion stoppers and crimped caps.

1. A compound of the formula 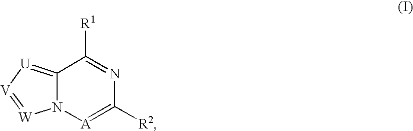
in which
either
U represents N,
V represents CR¹²,
W represents N,
A represents CR¹⁵,
or
U represents N,
V represents N,
W represents CR¹⁶,
A represents N.
where
R¹² represents hydrogen, hydroxyl, amino, hydroxycarbonyl, aminocarbonyl, trifluoromethyl, trifluoromethoxy, cyano, C₁-C₄-alkyl, C₁-C₄-alkoxy, C₁-C₄-alkylamino, C₁-C₄-alkylcarbonyl, C₁-C₄-alkoxycarbonyl, C₁-C₄-alkylaminocarbonyl, C₁-C₄-alkylcarbonylamino, C₁-C₄-alkylsulphonyl-amino, 5- or 6-membered heterocyclylcarbonyl, —CH₂R¹³ or —CH₂CH₂R¹⁴, where heterocyclylcarbonyl is substituted by 1 to 3 substituents, where the substituents independently of one another are selected from the group consisting of halogen, oxo, C₁-C₄-alkyl, C₁-C₄-alkoxy, C₁-C₄-alkylamino, C₁-C₄-alkylcarbonyl, C₁-C₄-alkoxycarbonyl and C₁-C₄-alkylamino carbonyl, and
where alkoxy, alkylamino, alkylcarbonyl, alkoxycarbonyl, alkylaminocarbonyl, alkylcarbonylamino and alkylsulphonylamino may be substituted by a substituent, where the substituent is selected from the group consisting of hydroxyl, amino, hydroxycarbonyl, aminocarbonyl, C₁-C₄-alkoxy, C₁-C₄-alkylamino, C₁-C₄-alkoxycarbonyl, C₁-C₄-alkylamino-carbonyl, C₁-C₄-alkylcarbonyl-amino, 5- or 6-membered heterocyclyl and phenyl,
where phenyl may be substituted by 1 to 3 substituents, where the substituents independently of one another are selected from the group consisting of halogen, cyano, trifluoromethyl, trifluoromethoxy, aminocarbonyl, C₁-C₄-alkyl, C₁-C₄-alkoxy, C₁-C₄-alkylamino, C₁-C₄-alkylcarbonyl, C₁-C₄-alkoxycarbonyl, C₁-C₄-alkylaminocarbonyl and C₁-C₄-alkylcarbonylamino,
and
where heterocyclyl may be substituted by 1 to 3 substituents, where the substituents independently of one another are selected from the group consisting of halogen, oxo, C₁-C₄-alkyl, C₁-C₄-alkoxy, C₁-C₄-alkylamino, C₁-C₄-alkylcarbonyl, C₁-C₄-alkoxycarbonyl and C₁-C₄-alkylaminocarbonyl,
and
where
R¹³ represents hydroxyl, amino, cyano, hydroxycarbonyl, aminocarbonyl, C₁-C₄-alkoxy, C₁-C₄-alkylamino, C₁-C₄-alkoxy-carbonyl, C₁-C₄-alkylaminocarbonyl, C₁-C₄-alkylcarbonylamino or 5- or 6-membered heterocyclyl,
where alkoxy, alkylamino, alkoxycarbonyl, alkylaminocarbonyl and alkylcarbonylamino may be substituted by a substituent, where the substituent is selected from the group consisting of hydroxyl, amino, hydroxycarbonyl, aminocarbonyl, C₁-C₄-alkoxy, C₁-C₄-alkylamino, C₁-C₄-alkoxycarbonyl, C₁-C₄-alkylamino carbonyl and C₁-C₄-alkylcarbonylamino,
and
where heterocyclyl may be substituted by 1 to 3 substituents, where the substituents independently of one another are selected from the group consisting of halogen, oxo, C₁-C₄-alkyl, C₁-C₄-alkoxy, C₁-C₄-alkylamino, C₁-C₄-alkylcarbonyl, C₁-C₄-alkoxycarbonyl and C₁-C₄-alkylaminocarbonyl,
and
where
R¹⁴ represents hydroxyl, amino, cyano, hydroxycarbonyl, aminocarbonyl, C₁-C₄-alkoxy, C₁-C₄-alkylamino, C₁-C₄-alkoxy-carbonyl, C₁-C₄-alkylaminocarbonyl, C₁-C₄-alkylcarbonylamino or 5- or 6-membered heterocyclyl,
where alkoxy, alkylamino, alkoxycarbonyl, alkylaminocarbonyl and alkylcarbonylamino may be substituted by a substituent, where the substituent is selected from the group consisting of hydroxyl, amino, hydroxycarbonyl, aminocarbonyl, C₁-C₄-alkoxy, C₁-C₄-alkylamino, C₁-C₄-alkoxycarbonyl, C₁-C₄-alkylamino carbonyl and C₁-C₄-alkylcarbonylamino,
and
where heterocyclyl may be substituted by 1 to 3 substituents, where the substituents independently of one another are selected from the group consisting of halogen, oxo, C₁-C₄-alkyl, C₁-C₄-alkoxy, C₁-C₄-alkylamino, C₁-C₄-alkylcarbonyl, C₁-C₄-alkoxycarbonyl and C₁-C₄-alkylaminocarbonyl,
R¹⁵ represents hydrogen, halogen, cyano, trifluoromethyl, C₁-C₃-alkyl, methoxy, methylthio or cyclopropyl,
R¹⁶ represents hydrogen or methyl,
R¹ represents a group of the formula 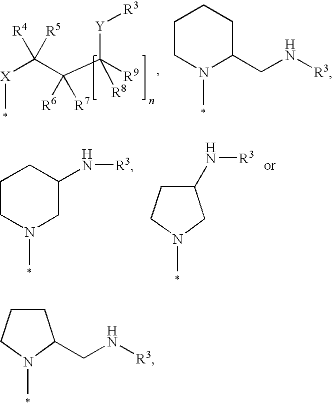
where
* is the point of attachment to the heterocycle,
n represents the number 0 or 1,
X represents NR¹⁰, S or O,
where
R¹⁰ represents hydrogen, C₁-C₃-alkyl or cyclopropyl,
Y represents NR¹¹ or S,
where
R¹¹ represents hydrogen, C₁-C₃-alkyl or cyclopropyl,
R³ represents pyrid-2-yl, pyrimid-2-yl, 2-aminopyrimid-4-yl, 2-(mono-C₁-C₄-alkylamino)pyrimid-4-yl, 2-(mono-C₃-C₄-cycloalkylamino)pyrimid-4-yl, pyridazin-3(2H)-on-6-yl, 1,3-oxazol-2-yl, 1,3-oxazol-4-yl, 1,2,4-oxadiazol-3-yl, 1,2,3-oxadiazol-4-yl, 1,3-thiazol-2-yl, 1,3-thiazol-4-yl, 1H-1,2,4-triazol-5-yl, 2,4-dihydro-3H-1,2,4-triazol-3-on-5-yl or 1,2-pyrazol-5-yl, where pyrid-2-yl, pyrimid-2-yl, 1,3-oxazol-2-yl, 1,3-oxazol-4-yl, 1,3-thiazol-2-yl and 1,3-thiazol-4-yl are substituted by 1 or 2 substituents, where the substituents independently of one another are selected from the group consisting of halogen, cyano, nitro, amino, trifluoromethyl, trifluoromethoxy, aminocarbonyl, trifluoromethylcarbonyl, C₁-C₄-alkyl, C₁-C₄-alkoxy, C₁-C₄-alkylamino, C₃-C₄-cycloalkylamino, C₁-C₄-alkylcarbonyl, C₁-C₄-alkoxycarbonyl, C₁-C₄-alkylaminocarbonyl and C₃-C₆-cycloalkylcarbonyl,
where alkyl, alkoxy, alkylamino, alkylcarbonyl, alkoxycarbonyl, alkylaminocarbonyl and cycloalkylcarbonyl may be substituted by a substituent, where the substituent is selected from the group consisting of halogen, cyano, hydroxyl, amino, trifluoromethyl and C₃-C₆-cycloalkyl,
and
where 2-aminopyrimid-4-yl, 2-(mono-C₁-C₄-alkylamino)pyrimid-4-yl, 2-(mono-C₃-C₄-cycloalkylamino)pyrimid-4-yl, pyridazin-3(2H)-on-6-yl, 1,2,4-oxadiazol-3-yl, 1,2,3-oxadiazol-4-yl, 1H-1,2,4-triazol-5-yl, 2,4-dihydro-3H-1,2,4-triazol-3-on-5-yl and 1,2-pyrazol-5-yl may be substituted by a substituent, where the substituent is selected from the group consisting of halogen, cyano, nitro, amino, trifluoromethyl, trifluoromethoxy, aminocarbonyl, trifluoromethylcarbonyl, C₁-C₄-alkyl, C₁-C₄-alkoxy, C₁-C₄-alkylamino, C₃-C₄-cycloalkylamino, C₁-C₄-alkoxycarbonyl, C₁-C₄-alkylaminocarbonyl and C₃-C₆-cycloalkylcarbonyl,
R⁴ represents hydrogen, C₁-C₃-alkyl or cyclopropyl,
R⁵ represents hydrogen or C₁-C₃-alkyl,
R⁶ represents hydrogen, C₁-C₃-alkyl or cyclopropyl,
R⁷ represents hydrogen or C₁-C₃-alkyl,
R⁸ represents hydrogen, C₁-C₃-alkyl or cyclopropyl,
R⁹ represents hydrogen or C₁-C₃-alkyl,
R² represents C₆-C₁₀-aryl or 5- to 10-membered heteroaryl,
where aryl and heteroaryl may be substituted by 1 to 3 substituents, where the substituents independently of one another are selected from the group consisting of hydroxyl, hydroxymethyl, amino, halogen, cyano, trifluoromethyl, trifluoromethoxy, aminocarbonyl, C₁-C₄-alkyl, C₁-C₄-alkoxy, C₁-C₄-alkoxymethyl, C₁-C₄-alkylamino, C₁-C₄-alkylaminomethyl, C₁-C₄-alkylcarbonyl, C₁-C₄-alkoxycarbonyl, C₁-C₄-alkylaminocarbonyl, C₁-C₄-alkylcarbonylamino, C₁-C₄-alkylsulphonyl, C₁-C₄-alkylsulphonylamino, C₁-C₄-alkylaminosulphonyl, phenyl, benzyloxy, 5- or 6-membered heterocyclyl, 5- or 6-membered heterocyclylcarbonyl, 5- or 6-membered heterocyclylmethyl and 5- or 6-membered heteroaryl,
where phenyl, benzyloxy, heterocyclyl, heterocyclylcarbonyl, heterocyclylmethyl and heteroaryl may be substituted by 1 to 3 substituents, where the substituents independently of one another are selected from the group consisting of halogen, cyano, trifluoromethyl, trifluoromethoxy, aminocarbonyl, C₁-C₄-alkyl, C₁-C₄-alkoxy, C₁-C₄-alkylamino, C₁-C₄-alkylcarbonyl, C₁-C₄-alkoxycarbonyl, C₁-C₄-alkylaminocarbonyl and C₁-C₄-alkylcarbonylamino,
or
two of the substituents on aryl together with the carbon atoms to which they are attached form a 1,3-dioxolane or 1,4-dioxane,
or one of its salts, its solvates or the solvates of its salts.

2. A compound according to claim 1, wherein either
U represents N,
V represents CR¹²,
W represents N,
A represents CR¹⁵,
or
U represents N,
V represents N,
W represents CR¹⁶,
A represents N.
where
R¹² represents hydrogen, hydroxycarbonyl, aminocarbonyl, C₁-C₄-alkyl, C₁-C₄-alkoxy, C₁-C₄-alkylamino, C₁-C₄-alkylcarbonyl, C₁-C₄-alkoxycarbonyl, C₁-C₄-alkylaminocarbonyl, C₁-C₄-alkylcarbonylamino, 5- or 6-membered heterocyclylcarbonyl, —CH₂R¹³ or —CH₂ CH₂R¹⁴,
where heterocyclylcarbonyl may be substituted by 1 to 3 substituents, where the substituents independently of one another are selected from the group consisting of oxo, C₁-C₄-alkyl, C₁-C₄-alkoxy, C₁-C₄-alkylamino, C₁-C₄-alkylcarbonyl, C₁-C₄-alkoxycarbonyl and C₁-C₄-alkylamino carbonyl, and
where alkoxy, alkylamino, alkylcarbonyl, alkoxycarbonyl, alkylaminocarbonyl and alkylcarbonylamino may be substituted by a substituent, where the substituent is selected from the group consisting of hydroxyl, amino, hydroxycarbonyl, aminocarbonyl, C₁-C₄-alkoxy, C₁-C₄-alkylamino and 5- or 6-membered heterocyclyl,
where heterocyclyl may be substituted by 1 or 2 substituents, where the substituents independently of one another are selected from the group consisting of oxo, C₁-C₄-alkyl, C₁-C₄-alkoxy, C₁-C₄-alkylamino, C₁-C₄-alkylcarbonyl, C₁-C₄-alkoxycarbonyl and C₁-C₄-alkylaminocarbonyl,
and
where
R¹³ represents hydroxyl, amino, hydroxycarbonyl, aminocarbonyl, C₁-C₄-alkoxy, C₁-C₄-alkylamino, C₁-C₄-alkoxycarbonyl, C₁-C₄-alkylaminocarbonyl, C₁-C₄-alkylcarbonylamino or 5- or 6-membered heterocyclyl,
where alkoxy, alkylamino, alkoxycarbonyl, alkylaminocarbonyl and alkylcarbonylamino may be substituted by a substituent, where the substituent is selected from the group consisting of hydroxyl, amino, hydroxycarbonyl, aminocarbonyl, C₁-C₄-alkoxy, C₁-C₄-alkylamino, C₁-C₄-alkoxycarbonyl, C₁-C₄-alkylamino carbonyl and C₁-C₄-alkylcarbonylamino,
and
where heterocyclyl may be substituted by 1 or 2 substituents, where the substituents independently of one another are selected from the group consisting of oxo, C₁-C₄-alkyl, C₁-C₄-alkoxy, C₁-C₄-alkylamino, C₁-C₄-alkylcarbonyl, C₁-C₄-alkoxycarbonyl and C₁-C₄-alkylaminocarbonyl,
and
where
R¹⁴ represents hydroxyl, amino, hydroxycarbonyl, aminocarbonyl, C₁-C₄-alkoxy, C₁-C₄-alkylamino, C₁-C₄-alkoxycarbonyl, C₁-C₄-alkylaminocarbonyl, C₁-C₄-alkylcarbonylamino or 5- or 6-membered heterocyclyl,
where alkoxy, alkylamino, alkoxycarbonyl, alkylaminocarbonyl and alkylcarbonylamino may be substituted by a substituent, where the substituent is selected from the group consisting of hydroxyl, amino, hydroxycarbonyl, aminocarbonyl, C₁-C₄-alkoxy, C₁-C₄-alkylamino, C₁-C₄-alkoxycarbonyl, C₁-C₄-alkylamino carbonyl and C₁-C₄-alkylcarbonylamino,
and
where heterocyclyl may be substituted by 1 or 2 substituents, where the substituents independently of one another are selected from the group consisting of oxo, C₁-C₄-alkyl, C₁-C₄-alkoxy, C₁-C₄-alkylamino, C₁-C₄-alkylcarbonyl, C₁-C₄-alkoxycarbonyl and C₁-C₄-alkylaminocarbonyl,
R¹⁵ represents hydrogen, halogen, cyano or trifluoromethyl,
R¹⁶ represents hydrogen or methyl,
R¹ represents a group of the formula 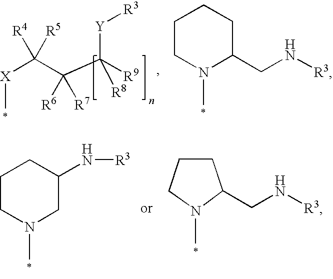
where
* is the point of attachment to the heterocycle,
n represents the number 0 or 1,
X represents NR¹⁰, S or O,
where
R¹⁰ represents hydrogen or methyl,
Y represents NR¹¹ or S,
where
R¹¹ represents hydrogen or methyl,
R³ represents pyrid-2-yl, pyrimid-2-yl, 2-aminopyrimid-4-yl, 1,3-oxazol-2-yl, 1,3-oxazol-4-yl, 1,2,4-oxadiazol-3-yl, 1,2,3-oxadiazol-4-yl, 1,3-thiazol-2-yl or 1,3-thiazol-4-yl,
where pyrid-2-yl, pyrimid-2-yl, 1,3-oxazol-2-yl, 1,3-oxazol-4-yl, 1,3-thiazol-2-yl and 1,3-thiazol-4-yl are substituted by 1 or 2 substituents,
where the substituents independently of one another are selected from the group consisting of halogen, cyano, nitro, amino, trifluoromethyl, trifluoromethoxy, aminocarbonyl, trifluoromethylcarbonyl, methyl, ethyl, methoxy, ethoxy, C₁-C₄-alkylamino, methylcarbonyl, ethylcarbonyl, cyclopropylcarbonyl, methoxycarbonyl and ethoxycarbonyl,
and
where 2-aminopyrimid-4-yl, 1,2,4-oxadiazol-3-yl and 1,2,3-oxadiazol-4-yl may be substituted by a substituent, where the substituent is selected from the group consisting of halogen, cyano, nitro, amino, trifluoromethyl, trifluoromethoxy, aminocarbonyl, trifluoromethylcarbonyl, methyl, ethyl, methoxy, ethoxy, C₁-C₄-alkylamino, methylcarbonyl, ethylcarbonyl, cyclopropylcarbonyl, methoxycarbonyl and ethoxycarbonyl,
R⁴ represents hydrogen or methyl,
R⁵ represents hydrogen or methyl,
R⁶ represents hydrogen or methyl,
R⁷ represents hydrogen or methyl,
R⁸ represents hydrogen or methyl,
R⁹ represents hydrogen or methyl,
R² represents C₆-C₁₀-aryl, thienyl, furyl, pyrrolyl, thiazolyl, oxazolyl, oxadiazolyl, pyrazolyl, imidazolyl, pyridyl, pyrimidyl, pyridazinyl, pyrazinyl, indolyl, indazolyl, quinolinyl, benzofuranyl or benzoxazolyl,
where aryl, thienyl, furyl, pyrrolyl, thiazolyl, oxazolyl, oxadiazolyl, pyrazolyl, imidazolyl, pyridyl, pyrimidyl, pyridazinyl, pyrazinyl, indolyl, indazolyl, quinolinyl, benzofuranyl and benzoxazolyl may be substituted by 1 to 3 substituents, where the substituents independently of one another are selected from the group consisting of hydroxyl, hydroxymethyl, amino, halogen, cyano, trifluoromethyl, trifluoromethoxy, aminocarbonyl, C₁-C₄-alkyl, C₁-C₄-alkoxy, C₁-C₄-alkoxymethyl, C₁-C₄-alkylamino, C₁-C₄-alkylaminomethyl, C₁-C₄-alkylcarbonyl, C₁-C₄-alkoxycarbonyl, C₁-C₄-alkylaminocarbonyl, C₁-C₄-alkylcarbonylamino, C₁-C₄-alkylsulphonyl, C₁-C₄-alkylsulphonylamino, C₁-C₄-alkylaminosulphonyl, phenyl, benzyloxy, 5- or 6-membered heterocyclyl, 5- or 6-membered heterocyclylcarbonyl, 5- or 6-membered heterocyclylmethyl and 5- or 6-membered heteroaryl,
where phenyl, benzyloxy, heterocyclyl, heterocyclylcarbonyl, heterocyclylmethyl and heteroaryl may be substituted by 1 to 3 substituents, where the substituents independently of one another are selected from the group consisting of halogen, cyano, trifluoromethyl, trifluoromethoxy, aminocarbonyl, C₁-C₄-alkyl, C₁-C₄-alkoxy, C₁-C₄-alkylamino, C₁-C₄-alkylcarbonyl, C₁-C₄-alkoxycarbonyl, C₁-C₄-alkylaminocarbonyl and C₁-C₄-alkylcarbonylamino,
or one of its salts, its solvates or the solvates of its salts.

3. A compound according to claim 1, wherein
either
U represents N,
V represents CR¹²,
W represents N.
A represents CR¹⁵,
or
U represents N,
V represents N,
W represents CR¹⁶,
A represents N,
where
R¹² represents hydrogen, hydroxycarbonyl, aminocarbonyl, methyl, ethyl, C₁-C₄-alkylcarbonyl, C₁-C₄-alkoxycarbonyl, C₁-C₄-alkylaminocarbonyl, C₁-C₄-alkylcarbonylamino, pyrrolidinylcarbonyl, piperidinylcarbonyl, piperazinylcarbonyl, mopholinylcarbonyl or —CH₂R¹³,
where pyrrolidinylcarbonyl, piperidinylcarbonyl, piperazinylcarbonyl and morpholinylcarbonyl may be substituted by 1 or 2 substituents, where the substituents independently of one another are selected from the group consisting of oxo, methyl and ethyl,
and
where alkylcarbonyl, C₂-C₄-alkoxycarbonyl and C₂-C₄-alkylaminocarbonyl may be substituted by a substituent, where the substituent is selected from the group consisting of hydroxyl, amino, C₁-C₄-alkylamino, pyrrolidinyl, piperidinyl, piperazinyl and morpholinyl,
where pyrrolidinyl, piperidinyl, piperazinyl and morpholinyl may be substituted by 1 or 2 substituents, where the substituents independently of one another are selected from the group consisting of oxo, methyl and ethyl,
and
where
R¹³ represents hydroxyl, amino, hydroxycarbonyl, aminocarbonyl, C₁-C₄-alkoxy, C₁-C₄-alkylamino, pyrrolidinyl, piperidinyl, piperazinyl or morpholinyl,
where pyrrolidinyl, piperidinyl, piperazinyl and morpholinyl may be substituted by 1 or 2 substituents, where the substituents independently of one another are selected from the group consisting of oxo, methyl and ethyl,
R¹⁵ represents hydrogen,
R¹⁶ represents hydrogen,
R¹ represents a group of the formula 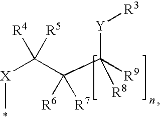
where
* is the point of attachment to the heterocycle,
n represents the number 0,
X represents NR¹⁰,
where
R¹⁰ represents hydrogen,
Y represents NR¹¹,
where
R¹¹ represents hydrogen,
R³ represents pyrid-2-yl, pyrimid-2-yl, 2-aminopyrimid-4-yl, 1,3-thiazol-2-yl or 1,3-thiazol-4-yl,
where pyrid-2-yl, pyrimid-2-yl, 1,3-thiazol-2-yl and 1,3-thiazol-4-yl are substituted by 1 or 2 substituents, where the substituents independently of one another are selected from the group consisting of fluorine, chlorine, cyano, nitro, amino, trifluoromethyl and trifluoromethylcarbonyl, and
where 2-aminopyrimid-4-yl may be substituted by a substituent, where the substituent is selected from the group consisting of fluorine, chlorine, cyano, nitro, amino, trifluoromethyl and trifluoromethylcarbonyl, represents hydrogen,
R⁴ represents hydrogen or methyl,
R⁵ represents hydrogen,
R⁶ represents hydrogen or methyl,
R⁸ represents hydrogen,
R⁹ represents hydrogen or methyl,
R² represents phenyl, thienyl, pyrazolyl or pyridyl,
where phenyl, thienyl, pyrazolyl and pyridyl may be substituted by 1 or 2 substituents, where the substituents independently of one another are selected from the group consisting of halogen, trifluoromethyl, trifluoromethoxy, aminocarbonyl, C₁-C₄-alkyl, C₁-C₄-alkoxy, C₁-C₄-alkoxycarbonyl, C₁-C₄-alkylaminocarbonyl, pyrrolidinyl, piperidinyl, morpholinyl and morpholinylcarbonyl,
or one of its salts, its solvates or the solvates of its salts.

4. A compound according to claim 1, wherein
either
U represents N,
V represents CR¹²,
W represents N,
A represents CR¹⁵,
or
U represents N,
V represents N,
W represents CR¹⁶,
A represents N,
where
R¹² represents hydrogen, hydroxycarbonyl, methyl, ethyl, methoxycarbonyl, ethoxycarbonyl, C₁-C₄-alkylaminocarbonyl, piperidinylcarbonyl or morpholinylcarbonyl,
where piperidinylcarbonyl and morpholinylcarbonyl may be substituted by a substituent, where the substituent is selected from the group consisting of methyl and ethyl,
and
where C₂-C₄-alkylaminocarbonyl may be substituted by a substituent, where the substituent is selected from the group consisting of C₁-C₄-alkylamino, piperazinyl and morpholinyl,
where piperazinyl and morpholinyl may be substituted by a substituent, where the substituent is selected from the group consisting of methyl and ethyl,
R¹⁵ represents hydrogen,
R¹⁶ represents hydrogen,
R¹ represents a group of the formula 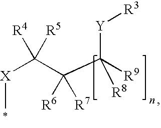
where
* is the point of attachment to the heterocycle,
n represents the number 0,
X represents NR¹⁰,
where
R¹⁰ represents hydrogen,
Y represents NR¹¹,
where
R¹¹ represents hydrogen,
R³ represents a group of the formula 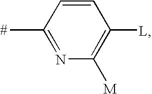
where
# is the point of attachment to Y,
L represents cyano, nitro, trifluoromethyl or trifluoromethylcarbonyl,
M represents hydrogen or amino,
R⁴ represents hydrogen,
R⁵ represents hydrogen or methyl,
R⁶ represents hydrogen,
R⁷ represents hydrogen or methyl,
R⁸ represents hydrogen,
R⁹ represents hydrogen,
R² represents phenyl,
where phenyl may be substituted by 1 or 2 substituents, where the substituents independently of one another are selected from the group consisting of fluorine, chlorine, trifluoromethyl, trifluoromethoxy, C₁-C₃-alkyl, methoxy, methoxycarbonyl and ethoxycarbonyl,
or one of its salts, its solvates or the solvates of its salts.

5. A compound according to claim 1, wherein
either
U represents N,
V represents CR¹²,
W represents N,
A represents CR¹⁵,
or
U represents N,
V represents N,
W represents CR¹⁶,
A represents N.
where
R¹² represents hydrogen,
R¹⁵ represents hydrogen,
R¹⁶ represents hydrogen,
R¹ represents a group of the formula 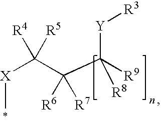
where
* is the point of attachment to the heterocycle,
n represents the number 0,
X represents NR¹⁰,
where
R¹⁰ represents hydrogen,
Y represents NR¹¹,
where
R¹¹ represents hydrogen,
R³ represents a group of the formula 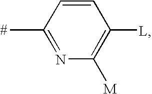
where
# is the point of attachment to Y,
either
L represents cyano,
and
M represents hydrogen,
or
L represents cyano, nitro or trifluoromethylcarbonyl,
and
M represents amino,
R⁴ represents hydrogen,
R⁵ represents hydrogen,
R⁶ represents hydrogen,
R⁷ represents hydrogen,
R⁸ represents hydrogen,
R⁹ represents hydrogen,
R² represents phenyl,
where phenyl is substituted by 1 or 2 substituents, where the substituents independently of one another are selected from the group consisting of fluorine, chlorine and trifluoromethyl,
or one of its salts, its solvates or the solvates of its salts.

6. A method of making a compound of the formula (I) or one of its salts, its solvates or the solvates of its salts according to claim 1, wherein
a compound of the formula 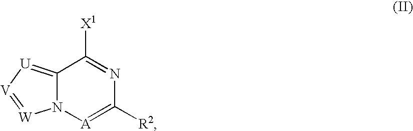
in which
A, U, V, W and R² have the meaning given in claim 1
and
X¹ represents halogen, preferably chlorine or fluorine,
is reacted with a compound of the formula
R¹—H (III),
in which
R¹ has the meaning given in claim 1.

7. A compound according to claim 1 for the treatment and/or prophylaxis of diseases.

8. (canceled)

9. (canceled)

10. A pharmaceutical composition, comprising a compound according to claim 1 in combination with an inert non-toxic pharmaceutically acceptable auxiliary.

11. The pharmaceutical composition according to claim 10 for the treatment and/or prophylaxis of haematological disorders.

12. A method for treating haematologic disorders in humans and animals by administering a therapeutically effective amount of at least one compound of claim 1.

13. (canceled)

14. A method for the ex vivo expansion of adult haematopoietic stem cells from bone marrow and/or from peripheral blood and/or for the ex vivo expansion of embryonal stem cells from umbilical cord blood, characterized in that an effective amount of a compound according to claim 1 is added.

15. A method for treating haematologic disorders in humans and animals by administering a therapeutically effective amount of a pharmaceutical composition according to claim 10.
